(19) United States
(12) Patent Application Publication
(10) Pub. No.: US 2024/0052033 A1
(43) Pub. Date: Feb. 15, 2024

(54) RECOMBINANT CD3 BINDING PROTEINS AND THEIR USE

(71) Applicant: MOLECULAR PARTNERS AG, Zürich-Schlieren (CH)

(72) Inventors: Sebastian GRIMM, Zürich-Schlieren (CH); Nina RESCHKE, Zürich-Schlieren (CH); Christian REICHEN, Zürich-Schlieren (CH); Bernd SCHLERETH, Zürich-Schlieren (CH); Victor LEVITSKY, Zürich-Schlieren (CH); Laura JEANBART, Zürich-Schlieren (CH)

(21) Appl. No.: 18/267,608

(22) PCT Filed: Dec. 16, 2021
(86) PCT No.: PCT/EP2021/086331

§ 371 Date: Jun. 15, 2023

Related U.S. Application Data
(60) Provisional application No. 63/126,356, filed on Dec. 16, 2020.
(60) Provisional application No. 63/182,394, filed on Apr. 30, 2021.

(30) Foreign Application Priority Data
Dec. 22, 2020 (EP) 20216705.2

(51) Int. Cl. C07K 16/28; C07K 14/47

(52) U.S. Cl. CPC C07K 16/2809; C07K 14/47; C07K 2318/20; C07K 2317/92; C07K 2317/94; C07K 2317/31; C07K 2317/35; A61K 2039/505

(57) ABSTRACT

The present invention relates to recombinant binding proteins comprising a designed ankyrin repeat domain with binding specificity for CD3. In addition, the invention relates to nucleic acids encoding such binding proteins, pharmaceutical compositions comprising such binding proteins or nucleic acids, and the use of such binding proteins, nucleic acids or pharmaceutical compositions in methods of disease-localized activation of T cells, and in methods of treating diseases, such as infectious diseases or cancer, in a mammal, including a human.

### CROSS-REFERENCE TO RELATED APPLICATIONS

The present application claims the benefit of priority to U.S. 63/126,356, filed on Dec. 16, 2020; EP20216705, filed on 22 Dec. 2020; and U.S. 63/182,394, filed on Apr. 30, 2021. The disclosures of these patent applications are incorporated herein for all purposes by reference in their entirety.

### FIELD OF THE DISCLOSURE

The present invention relates to recombinant binding proteins comprising a designed ankyrin repeat domain with binding specificity for CD3. In addition, the invention relates to nucleic acids encoding such binding proteins, pharmaceutical compositions comprising such binding proteins or nucleic acids, and the use of such binding proteins, nucleic acids or pharmaceutical compositions in methods of disease-localized activation of T cells, and in methods of treating diseases, such as infectious diseases or cancer, in a mammal, including a human.

### BACKGROUND

T cells play important roles in the adaptive immune response and specifically recognize foreign proteins in infected or cancerous cells. This recognition takes place through T cell receptors (TCR) that bind to foreign protein antigens displayed by major histocompatibility complexes (MHCs). Although TCR ligands typically bind relatively weakly, even a small number of ligands is sufficient to fully activate a T cell (Birnbaum et al, Proc Natl Acad Sci USA; 111(49):17576-81 (2014); Davis et al, Annu Rev Immunol; 16:523-44 (1998)). The T cell receptor does not signal on its own but it is non-covalently associated with a multi-subunit signaling apparatus typically comprising the CD3εγ and CD3εδ heterodimers and the CD3ζζ homodimer, which collectively form the TCR/CD3 complex. Due to its key role in the immune adaptive response, this multi-protein TCR/CD3 complex has attracted a remarkable interest for the development of new therapeutic tools in cancer immunotherapy. Among these tools are T cell engagers.

A T cell engager (TCE) is a protein that simultaneously binds to a target antigen, e.g., on a tumor cell, and to the TCR/CD3 complex on a T cell, triggering T cell activation by bypassing the normal TCR-MHC interaction (Ellerman, Methods; 154:102-117 (2019)). Examples of TCEs are the BiTE® molecules (Amgen).

Currently, T cell engagers typically have the form of bi-specific antibodies or antibody fragments, directed against a constant component of the TCR/CD3 complex, such as CD3, and a tumor-associated antigen (TAA). However, these T cell engaging therapeutic tools present several disadvantages, such as high production costs and the inability to target multiple disease-associated antigens, while also carrying the risk of causing certain severe side effects, such as cytokine release syndrome (CRS) (Shimabukuro-Vornhagen et al, J Immunother Cancer, 6(1):56 (2018); Labrjin et al, Nat Rev Drug Discov;18(8):585-608 (2019)) and on target/off tumor toxicities (Weiner et al, Cancer Res.; 55 (20): 4586-4593 (1995); Weiner et al, Cancer Immunol. Immunother; 42 (3); 141-150 (1996)). For instance, a TCE targeting EGFR was tested in non-human primates with safety findings related to engagement with normal tissue expressing lower levels of the antigen (Lutterbuese et al, Proc Nati Acad Sci USA; 107(28):12605-10 (2010)). Additionally, high affinity CD3-binders of HER-targeting TCEs were shown to distribute preferentially to secondary lymphatic tissues, reducing systemic exposure (Mandikian et al, Mol. Cancer Ther.; 17 (4): 776 LP-785 (2018)). Antibody-based T cell engagers often show more than 1000-fold higher affinity for CD3, when compared to the natural TCR-MHC interaction (Wu et al, Pharmacol Ther, 182:161-75 (2018); WO2014/167022; Junntila et al, Cancer Res.; 19:5561-71 (2014); Yang et al, J Immunol Aug.; 15 (137):1097-100 (1986)). This high affinity is correlated to lower efficiency in terms of T cell activation and tumor cell killing (Bortoletto et al, Eur. J. Immunol. 32; 11: 3102-3107 (2002); Ellerman, Methods; 154:102-117(2019); Mandikian et al, Mol. Cancer Ther.; 17 (4): 776 LP-785 (2018); Vafa et al, Frontiers in Oncology; 10: 446 (2020)).

Thus, there remains a need for new CD3-specific binding proteins with beneficial properties suitable for being used in T cell engager formats, and for therapeutic approaches for the treatment of diseases, including cancer and infectious diseases, benefitting from the CD3-specific binding.

### SUMMARY

The present invention provides recombinant binding proteins comprising a designed ankyrin repeat domain with binding specificity for CD3. Further provided are such binding proteins linked to one or more binding agents, preferably ankyrin repeat domains, with binding specificity for a Disease-Associated Antigen (DAA), such as, e.g., an Infection Associated Antigen (IAA), preferably a Virus Associated Antigen (VAA), or a Tumor Associated Antigen (TAA), which facilitate DAA-dependent activation of T cells by the binding proteins. In addition, the invention provides nucleic acids encoding such binding proteins and pharmaceutical compositions comprising such binding proteins or nucleic acids. The invention also provides the use of such binding proteins, nucleic acids or pharmaceutical compositions in methods for localized activation of T cells, e.g., tumor-localized or infection-localized activation, and in methods of treating diseases, such as infectious diseases, preferably viral infectious diseases, or cancer, in a mammal, including a human.

Applicant carried out dedicated studies to design, generate and select CD3-specific binding domains suitable to be used in T cell engager pharmaceuticals and having beneficial properties (see below). As a result of a complex screening process followed by several rounds of affinity maturation combined with rational design, four CD3-specific ankyrin repeat proteins were engineered, namely DARPin® protein #1 (SEQ ID NO: 1), DARPin® protein #2 (SEQ ID NO: 2), DARPin® protein #3 (SEQ ID NO: 3) and DARPin® protein #4 (SEQ ID NO: 4). These recombinant binding proteins all comprise a designed ankyrin repeat domain with binding specificity for CD3 while displaying different binding affinities to CD3 and/or to T cells. The binding proteins have a binding affinity to CD3 and/or to T cells that is adequately high to achieve targeted T cell activation, and at the same time relatively low so that the risk of adverse effects (e.g. caused by too much T cell activation) is reduced. These recombinant binding proteins were found to be suitable to be linked to one or more DAA-specific binding agent(s) to form functional T cell engager (TCE) molecules, including multi-specific TCEs. Thus, binding proteins of the invention may further comprise a binding agent with binding specificity for a disease-associated antigen. Such binding proteins in TCE format are capable of engaging the immune system, and more specifically T cells, in a localized and targeted fashion dependent on the presence of the respective DAA on disease-associated cells, such as tumor cells or infected cells. The binding proteins of the invention comprising an ankyrin repeat domain with binding specificity for CD3 (such as SEQ ID NO: 1, 2, 3 or 4 or variants thereof) thus represent a “toolbox” of CD3-specific binding proteins to be used, inter alia, in the generation of TCE drug candidates and in methods of treating diseases in human involving efficient activation of T cells while avoiding or reducing overstimulation of T cells and/or adverse effects.

In one aspect, the invention provides a recombinant binding protein comprising an ankyrin repeat domain with binding specificity for CD3, wherein said ankyrin repeat domain comprises an ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids. In one embodiment, said ankyrin repeat module is a first ankyrin repeat module and wherein said ankyrin repeat domain further comprises a second ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids. In one particular embodiment, said first ankyrin repeat module comprises an amino acid sequence selected from the group consisting of (1) SEQ ID NO: 5 and (2) sequences in which up to 9 amino acids in SEQ ID NO: 5 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids. In another particular embodiment, said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids. In a further particular embodiment, said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 8 and (2) sequences in which up to 9 amino acids of SEQ ID NO: 8 are substituted by other amino acids. In a further particular embodiment, said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 9 and (2) sequences in which up to 9 amino acids of SEQ ID NO: 9 are substituted by other amino acids. In one preferred embodiment, said first ankyrin repeat module is located N-terminally of said second ankyrin repeat module within said ankyrin repeat domain.

In one aspect, the invention provides a recombinant binding protein comprising an ankyrin repeat domain with binding specificity for CD3, wherein said ankyrin repeat domain comprises an amino acid sequence with at least 80% amino acid sequence identity with any one of SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N.

In one embodiment, any of said binding proteins binds human CD3 in PBS with a dissociation constant (K_{D}) below 10⁻⁷ M. In a further embodiment, any of said binding proteins binds human CD3 on T cells with an EC₅₀ ranging from 2 to 900 nM, preferably from about 5 to about 400 nM. In a further embodiment, said ankyrin repeat domain with binding specificity for CD3 comprised in said binding proteins has a melting temperature (Tₘ) higher than 65° C., preferably higher than 70° C. In a further embodiment, said ankyrin repeat domain with binding specificity for CD3 comprised in said binding proteins has an aggregation onset temperature (T_{agg}) higher than 70° C., preferably higher than 75° C. In one particular embodiment, said ankyrin repeat domain with binding specificity for CD3 comprises an amino acid sequence with at least 80% amino acid sequence identity with SEQ ID NO: 1 and said binding protein binds human CD3 in PBS with a dissociation constant (K_{D}) below 10⁻⁷ M and/or binds human CD3 on T cells with an EC₅₀ ranging from 2 to 900 nM, preferably from about 5 to about 400 nM. In one particular embodiment, said ankyrin repeat domain with binding specificity for CD3 comprises an amino acid sequence with at least 80% amino acid sequence identity with SEQ ID NO: 1, and wherein said ankyrin repeat domain has a melting temperature (Tₘ) higher than 75° C., preferably higher than 80° C., and/or said ankyrin repeat domain has an aggregation onset temperature (T_{agg}) higher than 75° C., preferably higher than 80° C., and/or said binding protein binds human CD3 in PBS with a dissociation constant (K_{D}) below 10⁻⁷ M, and/or said binding protein binds human CD3 on T cells with an EC₅₀ ranging from 2 to 900 nM, preferably from about 5 to about 400 nM. In another particular embodiment, said ankyrin repeat domain with binding specificity for CD3 comprises an amino acid sequence with at least 80% amino acid sequence identity with SEQ ID NO: 2 and said binding protein binds human CD3 in PBS with a dissociation constant (K_{D}) below 10⁻⁷ M, preferably of or below about 5×10⁻⁸ M, and/or binds human CD3 on T cells with an EC₅₀ ranging from 2 to 900 nM, preferably from about 250 to about 400 nM. In another particular embodiment, said ankyrin repeat domain with binding specificity for CD3 comprises an amino acid sequence with at least 80% amino acid sequence identity with SEQ ID NO: 2, and wherein said ankyrin repeat domain has a melting temperature (Tₘ) higher than 72° C., preferably higher than 77° C., and/or said ankyrin repeat domain has an aggregation onset temperature (T_{agg}) higher than 70° C., preferably higher than 75° C., and/or said binding protein binds human CD3 in PBS with a dissociation constant (K_{D}) below 10⁻⁷ M, preferably of or below about 5×10⁻⁸ M, and/or said binding protein binds human CD3 on T cells with an EC₅₀ ranging from 2 to 900 nM, preferably from about 250 to about 400 nM. In an alternative particular embodiment, said ankyrin repeat domain with binding specificity for CD3 comprises an amino acid sequence with at least 80% amino acid sequence identity with SEQ ID NO: 3 and said binding protein binds human CD3 in PBS with a dissociation constant (K_{D}) below 10⁻⁷ M, preferably of or below about 2×10⁻⁸ M, and/or binds human CD3 on T cells with an EC₅₀ ranging from 2 to 900 nM, preferably from about 50 to about 100 nM. In another particular embodiment, said ankyrin repeat domain with binding specificity for CD3 comprises an amino acid sequence with at least 80% amino acid sequence identity with SEQ ID NO: 3, and wherein said ankyrin repeat domain has a melting temperature (Tₘ) higher than 65° C., preferably higher than 70° C., and/or said ankyrin repeat domain has an aggregation onset temperature (T_{agg}) higher than 70° C., preferably higher than 75° C., and/or said binding protein binds human CD3 in PBS with a dissociation constant (K_{D}) below 10⁻⁷ M, preferably of or below about 2×10⁻⁸ M, and/or said binding protein binds human CD3 on T cells with an EC₅₀ ranging from 2 to 900 nM, preferably from about 50 to about 100 nM. In another alternative embodiment, said ankyrin repeat domain with binding specificity for CD3 comprises an amino acid sequence with at least 80% amino acid sequence identity with SEQ ID NO: 4 and said binding protein binds human CD3 in PBS with a dissociation constant (K_{D}) below 10⁻⁷ M, preferably of or below about 10⁻⁸ M and/or binds human CD3 on T cells with an EC₅₀ ranging from 2 to 900 nM, preferably from about 5 to about 15 nM. In another particular embodiment, said ankyrin repeat domain with binding specificity for CD3 comprises an amino acid sequence with at least 80% amino acid sequence identity with SEQ ID NO: 4, and wherein said ankyrin repeat domain has a melting temperature (Tₘ) higher than 73° C., preferably higher than 78° C., and/or said ankyrin repeat domain has an aggregation onset temperature (T_{agg}) higher than 70° C., preferably higher than 75° C., and/or said binding protein binds human CD3 in PBS with a dissociation constant (K_{D}) below 10⁻⁷ M, preferably of or below about 10⁻⁸ M, and/or said binding protein binds human CD3 on T cells with an EC₅₀ ranging from 2 to 900 nM, preferably from about 5 to about 15 nM.

In another aspect, the invention provides a recombinant binding protein comprising an ankyrin repeat domain with binding specificity for CD3, wherein said binding protein further comprises a binding agent with binding specificity for a disease-associated antigen. In one embodiment, said binding agent is a designed ankyrin repeat domain with binding specificity for a disease-associated antigen. In a further embodiment, said disease-associated antigen is an Infection Associated Antigen (IAA), preferably a Virus Associated Antigen (VAA). In another embodiment, said disease-associated antigen is Tumor Associated Antigen (TAA). In one aspect of the invention, the binding agent with binding specificity for a disease-associated antigen is covalently linked to or fused to the designed ankyrin repeat domain with binding specificity for CD3. In one particular embodiment, the binding agent with binding specificity for a disease-associated antigen is covalently linked to the ankyrin repeat domain with binding specificity for CD3 with a peptide linker, preferably a proline-threonine rich peptide linker. In one embodiment, the amino acid sequence of said peptide linker has a length from 1 to 50 amino acids, preferably from 6 to 38 amino acids. In one preferred embodiment, said binding agent with binding specificity for a disease-associated antigen is located N-terminally of said ankyrin repeat domain with binding specificity for CD3 within said binding protein.

In another aspect, the invention provides a recombinant binding protein comprising an ankyrin repeat domain with binding specificity for CD3, wherein said binding protein further comprises a half-life extending moiety. In one embodiment, said half-life extending moiety comprises a binding agent with binding specificity for human serum albumin. In one embodiment, said binding agent with binding specificity for human serum albumin is a designed ankyrin repeat domain with binding specificity for human serum albumin. In one particular embodiment, said binding agent with binding specificity for human serum albumin is a designed ankyrin repeat domain with binding specificity for human serum albumin comprising the amino acid sequence of any one of SEQ ID NOs: 28 to 30, preferably SEQ ID NO: 29. In one preferred embodiment, said half-life extending moiety is located N-terminally of said ankyrin repeat domain with binding specificity for CD3 within said binding protein.

In another aspect, the invention provides nucleic acids encoding the recombinant binding proteins of the invention and pharmaceutical compositions comprising the binding protein of the invention or the nucleic acid of the invention and optionally a pharmaceutically acceptable carrier and/or diluent.

In another aspect, the invention provides the binding protein of the invention, the nucleic acid of the invention or the pharmaceutical composition of the invention for use in a method of treating a medical condition. In one embodiment, said medical condition is an infectious disease, preferably a viral infectious disease. In another embodiment, said medical condition is a cancer.

In another aspect, the invention provides a method of tumor-localized activation of T cells in a mammal, preferably a human, the method comprising administering to said mammal the binding protein of the invention, the nucleic acid of the invention or the pharmaceutical composition of the invention. In one particular embodiment, such method comprises administering the binding protein of the invention further comprising a binding agent with binding specificity for a tumor-associated antigen to a mammal, including a human patient with a tumor, resulting in localized T-cell activation in the tumor tissue.

In another aspect, the invention provides a method of infection-localized activation of T cells in a mammal, preferably a human, the method comprising administering to said mammal the binding protein of the invention, the nucleic acid of the invention or the pharmaceutical composition of the invention. In one particular embodiment, such method comprises administering the binding protein of the invention further comprising a binding agent with binding specificity for a virus-associated antigen to a mammal, including a human patient with a viral infection, resulting in localized T-cell activation in the infected tissue.

In another aspect, the invention provides a method for treating a medical condition in a human patient, the method comprising administering to said patient a therapeutically effective amount of the binding protein of the invention, the nucleic acid of the invention or the pharmaceutical composition of the invention. In one particular embodiment, such method comprises administering to said patient a therapeutically effective amount of a binding protein of the invention further comprising a binding agent with binding specificity for a disease-associated antigen, a nucleic acid encoding such a binding protein of the invention or a pharmaceutical composition comprising such a binding protein of the invention. In one embodiment said medical condition is cancer. In another embodiment, said medical condition is an infectious disease, preferably a viral infectious disease.

In one particular embodiment, the medical condition is cancer, wherein the cancer or tumor tissue comprises cells that express or display a tumor-associated antigen, and said binding agent with binding specificity for a disease-associated antigen binds said tumor-associated antigen expressed or displayed in said cells. In one particular embodiment, said disease-associated antigen is the extracellular domain of a cell surface protein expressed or overexpressed in said cancer or tumor tissue. In another particular embodiment, said binding agent with binding specificity for a tumor-associated antigen binds to a peptide-MHC complex, wherein said peptide is derived from a protein expressed in a tumor cell (such as, e.g. NY-ESO-1 or MAGE-A3). In one particular embodiment, said MHC is MHC class I. In one embodiment, said cancer is selected from carcinoma, sarcoma, myeloma, leukemia, lymphoma and mixed type-cancers.

In another particular embodiment, the medical condition is an infectious disease, preferably a viral infectious disease, wherein the infected tissue comprises cells that express or display an infection-associated antigen, preferably a virus-associated antigen, and said binding agent with binding specificity for a disease-associated antigen binds said infection-associated antigen expressed or displayed in said cells. In one particular embodiment, said disease-associated antigen is the extracellular domain of a virus protein expressed or overexpressed in said infected tissue. In another particular embodiment, said binding agent with binding specificity for a virus-associated antigen binds to a peptide-MHC complex, wherein said peptide is derived from a protein of an infectious agent, such as a bacterial infectious agent or a viral infectious agent, preferably a viral infectious agent (such as, e.g. HBcAg or EBNA-1). In one particular, embodiment, said MHC is MHC class I.

The invention further provides a kit comprising the recombinant binding protein of the invention, the nucleic acid of the invention or the pharmaceutical composition of the invention. The invention further provides a method for producing the recombinant binding protein of the invention, the method comprising the steps of (i) expressing said recombinant binding protein in a suitable host cell (prokaryotic or eukaryotic cell), and (ii) purifying said recombinant binding protein (e.g., using chromatography).

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A recombinant binding protein comprising an ankyrin repeat domain with binding specificity for CD3, wherein said ankyrin repeat domain comprises an ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids.

E2. The binding protein of E1, wherein said ankyrin repeat module is a first ankyrin repeat module and wherein said ankyrin repeat domain further comprises a second ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids.

E3. The binding protein of E2, wherein said first ankyrin repeat module comprises an amino acid sequence selected from the group consisting of (1) SEQ ID NO: 5 and (2) sequences in which up to 9 amino acids in SEQ ID NO: 5 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids.

E4. The binding protein of E2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids.

E5. The binding protein of E2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 8 and (2) sequences in which up to 9 amino acids of SEQ ID NO: 8 are substituted by other amino acids.

E6. The binding protein of E2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 9 and (2) sequences in which up to 9 amino acids of SEQ ID NO: 9 are substituted by other amino acids.

E7. The binding protein of any of E2 to E6, wherein said first ankyrin repeat module is located N-terminally of said second ankyrin repeat module within said ankyrin repeat domain.

E8. A recombinant binding protein comprising an ankyrin repeat domain with binding specificity for CD3, wherein said ankyrin repeat domain comprises an amino acid sequence with at least 80% amino acid sequence identity with any one of SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N.

E9. The binding protein of any of E1 to E8, wherein said binding protein binds human CD3 in PBS with a dissociation constant (K_{D}) below 10⁻⁷ M.

E10. The binding protein of any of E1 to E9, wherein said binding protein binds human CD3 on T cells with an EC₅₀ ranging from 2 to 900 nM.

E11. The binding protein of any of E1 to E10, wherein said ankyrin repeat domain with binding specificity for CD3 comprises an amino acid sequence with at least 80% amino acid sequence identity with SEQ ID NO: 1.

E12. The binding protein of any of E1 to E10, wherein said ankyrin repeat domain with binding specificity for CD3 comprises an amino acid sequence with at least 80% amino acid sequence identity with SEQ ID NO: 2.

E13. The binding protein of E12, wherein said binding protein binds human CD3 in PBS with a dissociation constant (K_{D}) of or below about 5×10⁻⁸ M.

E14. The binding protein of E12 or E13, wherein said binding protein binds human CD3 on T cells with an EC₅₀ ranging from about 250 to about 400 nM.

E15. The binding protein of any of E1 to E10, wherein said ankyrin repeat domain with binding specificity for CD3 comprises an amino acid sequence with at least 80% amino acid sequence identity with SEQ ID NO: 3.

E16. The binding protein of E15, wherein said binding protein binds human CD3 in PBS with a dissociation constant (K_{D}) of or below about 2×10⁻⁸ M.

E17. The binding protein of E15 or E16, wherein said binding protein binds human CD3 on T cells with an EC₅₀ ranging from about 50 to about 100 nM.

E18. The binding protein of any of E1 to E10, wherein said ankyrin repeat domain with binding specificity for CD3 comprises an amino acid sequence with at least 80% amino acid sequence identity with SEQ ID NO: 4.

E19. The binding protein of E18, wherein said binding protein binds human CD3 in PBS with a dissociation constant (K_{D}) of or below about 10⁻⁸ M.

E20. The binding protein of E18 or E19, wherein said binding protein binds human CD3 on T cells with an EC₅₀ ranging from about 5 to about 15 nM.

E21. The binding protein of any of E1 to E20, wherein said binding protein further comprises a binding agent with binding specificity for a disease-associated antigen.

E22. The binding protein of E21, wherein said binding agent is an ankyrin repeat domain with binding specificity for a disease-associated antigen.

E23. The binding protein of E21 or E22, wherein said disease-associated antigen is an Infection Associated Antigen (IAA), preferably a Virus Associated Antigen (VAA), or a Tumor Associated Antigen (TAA).

E24. The binding protein of any one of E21 to E23, wherein said binding agent with binding specificity for a disease-associated antigen is located N-terminally of said ankyrin repeat domain with binding specificity for CD3 within said binding protein.

E25. The binding protein of any one of E21 to E24, wherein said binding agent with binding specificity for a disease-associated antigen is covalently linked to said ankyrin repeat domain with binding specificity for CD3 with a peptide linker.

E26. The binding protein of E25, wherein said peptide linker is a proline-threonine rich peptide linker.

E27. The binding protein of E25 or E26, wherein the amino acid sequence of said peptide linker has a length from 1 to 50 amino acids, preferably from 6 to 38 amino acids.

E28. The binding protein of any of E1 to E27, wherein said binding protein further comprises a half-life extending moiety.

E29. The binding protein of E28, wherein said half-life extending moiety comprises a binding agent with binding specificity for human serum albumin.

E30. The binding protein of E29, wherein said binding agent with binding specificity for human serum albumin is an ankyrin repeat domain comprising an amino acid sequence of any one of SEQ ID NOs: 28 to 30, preferably SEQ ID NO: 29.

E31. The binding protein of any one of E28 to E30, wherein said half-life extending moiety is located N-terminally of said ankyrin repeat domain with binding specificity for CD3 within said binding protein.

E32. A nucleic acid encoding the binding protein of any one of E1 to E31.

E33. A pharmaceutical composition comprising the binding protein of any one of E1 to E31 or the nucleic acid of E32, and optionally a pharmaceutically acceptable carrier and/or diluent.

E34. The binding protein of any one of E1 to E31, the nucleic acid of E32 or the pharmaceutical composition of E33 for use in a method of treating a medical condition.

E35. The binding protein of E34, wherein the medical condition is an infectious disease, preferably a viral infectious disease, or a cancer.

E36. A method of tumor-localized activation of T cells in a mammal, including a human, the method comprising the step of administering to said mammal the binding protein of any one of E1 to E31, the nucleic acid of E32 or the pharmaceutical composition of E33.

E37. A method of infection-localized activation of T cells in a mammal, including a human, the method comprising the step of administering to said mammal the binding protein of any one of E1 to E31, the nucleic acid of E32 or the pharmaceutical composition of E33.

E38. A method of treating a medical condition, the method comprising the step of administering to a patient in need thereof a therapeutically effective amount of the binding protein of any one of E1 to E31, the nucleic acid of E32 or the pharmaceutical composition of E33.

E39. The method of E38, wherein said medical condition is an infectious disease, preferably a viral infectious disease, or a cancer.

### BRIEF DESCRIPTION OF THE FIGURES

FIG. 1: SDS-PAGE gel analysis of the purification of four selected ankyrin repeat proteins with binding specificity for human CD3, DARPin® protein #1, DARPin® protein #2, DARPin® protein #3 and DARPin® protein #4. M corresponds to a protein size marker (reduced SDS-PAGE, NuPAGE 4-12%, Bis Tris (invitrogen) gel; 5 μg/lane; MES-buffer; Instant blue staining). The molecular weights (kDa) of the marker proteins are indicated. Lane 1: protein size marker; Lane 2: purified DARPin® protein #1; Lane 3: purified DARPin® protein #2, Lane 4: purified DARPin® protein #3, Lane 5: purified DARPin® protein #4.

FIG. 2A-D. Stability assessment of exemplary designed ankyrin repeat proteins using UV spectroscopy. DARPin® proteins #1-4 were diluted 1:1 in TBS pH 8.0 (50 mM Tris, 500 mM NaCl) and mixed well prior to UV-VIS measurement by Nanodrop. The samples were assessed at −70° C. vs. heat-stressed for 18 days at 50° C. No absorption visible at 320 nm (indication of precipitation). No difference in spectra of reference and stressed samples.

FIG. 3. Stability assessment of exemplary designed ankyrin repeat proteins using reducing SDS-PAGE; 10% Bis-Tris gel; 5 μg/lane; MES-buffer; Instant blue staining. Expected MW: 14.5 kDa. Lane 1, M: Pre-stained marker—the molecular weights (kDa) of the marker proteins are indicated; lanes 2&3: DARPin® protein #1 at −70° C. vs. heat-stressed for 18 days at 50° C.; lanes 4&5: DARPin® protein #2 at −70° C. vs. heat-stressed for 18 days at 50° C.; lane 6&7: DARPin® protein #3 at −70° C. vs. heat-stressed for 18 days at 50° C.; lanes 8&9: DARPin® protein #4 at −70° C. vs. heat-stressed for 18 days at 50° C.

FIG. 4A-D. Stability assessment of exemplary designed ankyrin repeat proteins using size-exclusion chromatography (SEC). DARPin® #1-4 protein samples were diluted to 2 mg/ml and run in TBS pH 8.0 (50 mM Tris, 500 mM NaCl) over a GE Superdex 200 150/5 Increased column. Traces were analyzed at 280 nm. Black: Reference sample stored at −70° C.; grey: heat-stressed sample incubated at 50° C. for 18 days. The curves overlap to a large extent.

FIG. 5A-D. Thermal stability assessment of exemplary designed ankyrin repeat proteins using Circular Dichroism (CD) spectroscopy. Samples of DARPin® proteins #1-4 (A to D, respectively) were diluted to 2 μM in TBS pH 8.0 (50 mM Tris, 500 mM NaCl). Protein unfolding (forward) and refolding (reverse) was monitored. All proteins showed reversible unfolding with a Tm higher than 65° C. (indicated with dotted line in graphs).

FIG. 6A-D. Determination of the aggregation onset temperature Tagg with Dynamic Light Scattering (DLS). Samples of DARPin® proteins #1-4 were diluted to 1 mg/ml in TBS pH 8.0 (50 mM Tris, 500 mM NaCl) and Tagg determined by DLS (5 replicates, A1, A2, A3, A4, A5). All four tested ankyrin repeat proteins showed a high Tagg above 80° C.

FIG. 7A. Pharmacokinetic analysis of exemplary CD3-specific binding proteins in female BALB/c mice. The figure shows the group mean serum concentration-time profiles of DARPin® protein #5, DARPin® protein #6, DARPin® protein #7 and DARPin® protein #8 in female BALB/c mice (mean+/−max/min, N=3 per group), following single intravenous bolus administration of 1 mg/kg.

FIG. 7B: Surface Plasmon Resonance (SPR) analysis of binding proteins binding to human CD3, exemplified by DARPin® protein #3. Various concentrations (8, 16, 32, and 64 nM) of purified ankyrin repeat protein were applied to a GLC chip with immobilized human CD3 for on-rate and off-rate measurements. The obtained SPR trace analyses were used to determine the ankyrin repeat protein CD3 interaction. RU, Resonance Units; s, time in seconds.

FIG. 8A-B. CD3 binding of exemplified binding proteins to T cells. Binding to CD3 on Pan-T cells was assessed by Mirrorball. Shown are benchmark control molecules (known benchmark T cell engagers, TCE1 with binding specificity for TAA2 and TCE2 with binding specificity for TAA3) and selected ankyrin repeat proteins, DARPin® protein A, DARPin® protein B, DARPin® protein C and DARPin® protein D without (FIG. A) or with (FIG. B) half-life extension (HLE). Half-life extended proteins (B) show similar binding compared to the corresponding non-HLE molecules shown in (A). Pan-T cells from 5 different donors were tested, one representative donor is shown here. (*Negative control: a designed ankyrin repeat protein with binding specificity for TAA2 and TAA3 only, with and without half-life extension, respectively).

FIG. 9A-B. Short Term Target Cell Killing (LDH). Pan-T cells (as effector cells (E)) and TAA2/TAA3-expressing tumor cells (as target cells (T)) were incubated at an E:T ratio of 5:1 and tumor cell killing was assessed by LDH release in the supernatant after 48 hours of co-culture in the presence of serial dilutions of indicated molecules. Shown are benchmark control molecule (known benchmark T cell engager, TCE1 with binding specificity for TAA2) and selected ankyrin repeat proteins DARPin® protein A, DARPin® protein B, DARPin® protein C and DARPin® protein D without (FIG. A) or with (FIG. B) half-life extension (HLE). (A) For proteins without half-life extension, tumor cell killing induced by DARPin® protein B and DARPin® protein C is comparable to benchmark molecules, whereas DARPin® protein D and DARPin® protein A show lower potency in cytotoxicity. (B) Half-life extended proteins show 4-70-fold reduction in potency compared to the corresponding non-HLE molecules shown in (A). Pan-T cells from 5 different donors were tested, one representative donor is shown here. (*Negative control: a designed ankyrin repeat protein with binding specificity for TAA2 and TAA3, with or without half-life extension respectively).

FIG. 10A-B. Short term T cell activation measured by activation marker CD25. Pan-T and TAA2/TAA3-expressing tumor cells were incubated at an E:T ratio of 1:1 and T-cell activation assessed by FACS after 24 hours co-culture in the presence of serial dilutions of indicated molecules. Activated T-cells were gated as living CD8+/CD25+ cells. Shown are benchmark control molecule (known benchmark T cell engager, TCE1 with binding specificity for TAA2) and selected ankyrin repeat proteins DARPin® protein A, DARPin® protein B, DARPin® protein C and DARPin® protein D without (FIG. A) or with (FIG. B) half-life extension (HLE). (A) Without half-life extension, T-cell activation induced by DARPin® protein B and DARPin® protein C is comparable to the benchmark, whereas DARPin® protein D and DARPin® protein A show lower potencies. (B) Half-life extended proteins show 4-100-fold reduction in potency compared to the corresponding non-HLE molecules shown in (A). Pan-T cells from 7 different donors were tested, one representative donor is shown here. (*Negative control: a designed ankyrin repeat protein with binding specificity for TAA2 and TAA3 only, with or without half-life extension respectively).

FIG. 11A-B. Short term T cell activation measured by IFNγ secretion. Pan-T and TAA2/TAA3-expressing tumor cells were incubated at an E:T ratio of 1:1. After 24 hours co-culture in the presence of serial dilutions of indicated molecules, IFNγ secretion in culture supernatants was analyzed by ELISA. Shown are benchmark control molecule (known benchmark T cell engager, TCE1 with binding specificity for TAA2) and selected ankyrin repeat proteins DARPin® protein A, DARPin® protein B, DARPin® protein C and DARPin® protein D without (FIG. A) or with (FIG. B) half-life extension (HLE). (A) Without half-life extension, T-cell activation induced by DARPin® protein B and DARPin® protein C is comparable to benchmark molecules, whereas DARPin® protein D and v118 DARPin® protein A show lower potencies. (B) Half-life extended proteins show 3-20-fold reduction in potency compared to the corresponding non-HLE molecules shown in (A). Pan-T cells from 4 different donors were tested, one representative donor is shown here. (*Negative control: a designed ankyrin repeat protein with binding specificity for TAA2 and TAA3 only, with or without half-life extension respectively).

FIG. 12. Long-term tumor cell killing. Pan-T and TAA2/TAA3-expressing tumor cells were incubated at an E:T ratio of 5:1 and tumor cell killing assessed with an IncuCyte over 6 days of co-culture in the presence of serial dilutions of indicated molecules. Tumor cell killing is calculated as the ratio between area under the curve of Annexin V staining and cell proliferation. Shown are two benchmark control molecules (known benchmark T cell engagers, TCE1 with binding specificity for TAA2 and TCE2 with binding specificity for TAA3) and selected ankyrin repeat proteins DARPin® protein A, DARPin® protein B, DARPin® protein C and DARPin® protein D, without or with half-life extension (HLE). Data shows potent and specific tumor cell killing, comparable to benchmark molecules, with most tested proteins, independent of half-life extension. Only the lower-affinity DARPin® protein A shows a marked reduction of killing potency. Concentrations are, left to right, 2 nM and subsequently diluted 1/10 each for benchmark and tested proteins without HSA-binding domain, and 20 nM and subsequently diluted 1/10 each for tested proteins with HSA-binding domain. Pan-T cells from 5 different donors were used, one representative donor is shown here.

FIG. 13A-B. Long-term T-cell activation. Pan-T and TAA2/TAA3-expressing tumor cells were incubated at an E:T ratio of 1:1 and T-cell activation assessed by FACS after 5 days co-culture in the presence of serial dilutions of indicated molecules. Activated T-cells were gated as living CD8+/CD25+ cells. Shown are benchmark control molecules (known benchmark T cell engagers TCE1 with binding specificity for TAA2 and TCE2 with binding specificity for TAA3) and selected ankyrin repeat proteins DARPin® protein A, DARPin® protein B, DARPin® protein C and DARPin® protein D without or with half-life extension (HLE). (A) Without half-life extension, T-cell activation induced by DARPin® protein B and DARPin® protein C is comparable to benchmark molecules, whereas DARPin® protein A and DARPin® protein D show >100-fold reduction in potency. (B) Half-life extended proteins show >10-fold reduction in potency compared to the corresponding non-HLE molecules shown in (A). Pan-T cells from 2 different donors were used, one representative donor is shown here.

FIG. 14A-B. Long-term T-cell proliferation. Pan-T and TAA2/TAA3-expressing tumor cells were incubated at an E:T ratio of 1:1 and T-cell proliferation assessed by FACS after 5 days co-culture in the presence of serial dilutions of indicated molecules. Proliferating T-cells were gated as CellTrace Violet positive cells showing at least one cell division. Shown are two benchmark control molecules (known benchmark T cell engagers, TCE1 with binding specificity for TAA2 and TCE2 with binding specificity for TAA3) and selected ankyrin repeat proteins DARPin® protein A, DARPin® protein B, DARPin® protein C and DARPin® protein D without or with half-life extension (HLE). (A) Without half-life extension, T-cell proliferation induced by DARPin® protein B and DARPin® protein C is comparable to benchmark molecules, whereas DARPin® protein A and DARPin® protein D show >100-fold reduction in potency. (B) Half-life extended DARPin® proteins show >30-fold reduction in potency compared to the corresponding non-HLE molecules shown in (A). Pan-T cells from 2 different donors were used, one representative donor is shown here.

FIG. 15A-C. Tumor growth over time after tumor cell injection and treatment of mice. Treatment was done with vehicle, benchmark TAA3 T-cell engager, DARPin® protein #E, DARPin® protein #F, DARPin® protein #G and DARPin® protein #H as indicated and as described in Example 10.

FIG. 16A-B. (A) Tumor growth over time in mice injected intraperitoneally with hPBMC (n=5 mice per donor/2 hPBMC donors used), xenografted subcutaneously with TAA2/TAA3/TAA4 expressing tumor cells, and treated with PBS 1X (black circle), DARPin® protein #3 in multi-specific TCE format at 0.5 mg/kg (black square) or benchmark TAA3 T cell engager at 0.5 mg/kg (black triangle). Treatments were administered as described in Example 10. Data are presented in average+SEM. (B) Evaluation of tumor volume at day 17 after tumor cell xenograft.

### DETAILED DESCRIPTION OF THE INVENTION

As disclosed and exemplified herein, the disclosure provides ankyrin repeat proteins that specifically target CD3. Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four or more designed ankyrin repeat domains in one protein, resulting in binding proteins with two, three, four or more different specificities, make designed ankyrin repeat proteins ideal agonistic, antagonistic and/or inhibitory drug candidates and allow for novel drug designs, including, e.g., novel designs of T cell engager drug molecules. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs. DARPin® is a trademark owned by Molecular Partners AG, Switzerland.

In one aspect, the invention relates to a recombinant binding protein comprising an ankyrin repeat domain, wherein said ankyrin repeat domain has binding specificity for CD3, and wherein said ankyrin repeat domain comprises an ankyrin repeat module having an amino acid sequence selected from the group consisting of (1) SEQ ID NOs: 5 to 9 and (2) sequences in which up to 9, or up to 8, or up to 7, or up to 6, or up to 5, or up to 4, or up to 3, or up to 2, or up to 1 amino acids in any of SEQ ID NOs: 5 to 9 are substituted by other amino acids. Thus, in one embodiment, said ankyrin repeat module comprises an amino acid sequence selected from the group consisting of (1) SEQ ID NOs: 5 to 9 and (2) sequences in which up to 9 amino acids in any of SEQ ID NOs: 5 to 9 are substituted by other amino acids. In one embodiment, said ankyrin repeat module comprises an amino acid sequence selected from the group consisting of (1) SEQ ID NOs: 5 to 9 and (2) sequences in which up to 3 amino acids in any of SEQ ID NOs: 5 to 9 are substituted by other amino acids. In one embodiment, said ankyrin repeat module comprises an amino acid sequence selected from the group consisting of (1) SEQ ID NOs: 5 to 9 and (2) sequences in which up to 2 amino acids in any of SEQ ID NOs: 5 to 9 are substituted by other amino acids. In one embodiment, said ankyrin repeat module comprises an amino acid sequence selected from the group consisting of (1) SEQ ID NOs: 5 to 9 and (2) sequences in which up to 1 amino acid in any of SEQ ID NOs: 5 to 9 is substituted by another amino acid. In one embodiment, all of said 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 5 to 9.

In one embodiment, said ankyrin repeat module comprises the amino acid sequence of SEQ ID NO: 5 or a sequence in which one or two amino acids in SEQ ID NO: 5 are substituted by other amino acids. In one embodiment, said ankyrin repeat module comprises the amino acid sequence of SEQ ID NO: 6 or a sequence in which one or two amino acids in SEQ ID NO: 6 are substituted by other amino acids. In one embodiment, said ankyrin repeat module comprises the amino acid sequence of SEQ ID NO: 7 or a sequence in which one or two amino acids in SEQ ID NO: 7 are substituted by other amino acids. In one embodiment, said ankyrin repeat module comprises the amino acid sequence of SEQ ID NO: 8 or a sequence in which one or two amino acids in SEQ ID NO: 8 are substituted by other amino acids. In one embodiment, said ankyrin repeat module comprises the amino acid sequence of SEQ ID NO: 9 or a sequence in which one or two amino acids in SEQ ID NO: 9 are substituted by other amino acids. In one embodiment, said ankyrin repeat module comprises the amino acid sequence of SEQ ID NO: 5. In one embodiment, said ankyrin repeat module comprises the amino acid sequence of SEQ ID NO: 6 In one embodiment, said ankyrin repeat module comprises the amino acid sequence of SEQ ID NO: 7. In one embodiment, said ankyrin repeat module comprises the amino acid sequence of SEQ ID NO: 8. In one embodiment, said ankyrin repeat module comprises the amino acid sequence of SEQ ID NO: 9.

In one embodiment, said ankyrin repeat domain comprises a first ankyrin repeat module and a second ankyrin repeat module. In a preferred embodiment, said first ankyrin repeat module is located N-terminally of said second ankyrin repeat module within said ankyrin repeat domain. In one embodiment, said first and said second ankyrin repeat module each comprises an amino acid sequence selected from the group consisting of (1) SEQ ID NOs: 5 to 9 and (2) sequences in which up to 9, or up to 8, or up to 7, or up to 6, or up to 5, or up to 4, or up to 3, or up to 2, or up to 1 amino acids in any of SEQ ID NOs: 5 to 9 are substituted by other amino acids. Thus, in one embodiment, said ankyrin repeat domain comprises a first ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids and further comprises a second ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids.

In one particular embodiment, in such an ankyrin repeat domain said first ankyrin repeat module comprises an amino acid sequence selected from the group consisting of (1) SEQ ID NO: 5 and (2) sequences in which up to 9, or up to 8, or up to 7, or up to 6, or up to 5, or up to 4, or up to 3, or up to 2, or up to 1 amino acids in SEQ ID NO: 5 are substituted by other amino acids, and said second ankyrin repeat module comprises an amino acid sequence selected from the group consisting of (1) SEQ ID NO: 6 and (2) sequences in which up to 9, or up to 8, or up to 7, or up to 6, or up to 5, or up to 4, or up to 3, or up to 2, or up to 1 amino acids of SEQ ID NO: 6 are substituted by other amino acids.

Thus, in one embodiment, in such an ankyrin repeat domain said first ankyrin repeat module comprises an amino acid sequence selected from the group consisting of (1) SEQ ID NO: 5 and (2) sequences in which up to 9 amino acids in SEQ ID NO: 5 are substituted by other amino acids, and said second ankyrin repeat module comprises an amino acid sequence selected from the group consisting of (1) SEQ ID NO: 6 and (2) sequences in which up to 9 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which up to 6 amino acids in SEQ ID NO: 5 are substituted by other amino acids, and said second ankyrin repeat module comprises an amino acid sequence selected from the group consisting of (1) SEQ ID NO: 6 and (2) sequences in which up to 6 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which up to 5 amino acids in SEQ ID NO: 5 are substituted by other amino acids, and said second ankyrin repeat module comprises an amino acid sequence selected from the group consisting of (1) SEQ ID NO: 6 and (2) sequences in which up to 5 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which up to 4 amino acids in SEQ ID NO: 5 are substituted by other amino acids, and said second ankyrin repeat module comprises an amino acid sequence selected from the group consisting of (1) SEQ ID NO: 6 and (2) sequences in which up to 4 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which up to 3 amino acids in SEQ ID NO: 5 are substituted by other amino acids, and said second ankyrin repeat module comprises an amino acid sequence selected from the group consisting of (1) SEQ ID NO: 6 and (2) sequences in which up to 3 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which up to 2 amino acids in SEQ ID NO: 5 are substituted by other amino acids, and said second ankyrin repeat module comprises an amino acid sequence selected from the group consisting of (1) SEQ ID NO: 6 and (2) sequences in which up to 2 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 5 and (2) sequences in which 1 amino acid in SEQ ID NO: 5 is substituted by another amino acid, and said second ankyrin repeat module comprises an amino acid sequence selected from the group consisting of (1) SEQ ID NO: 6 and (2) sequences in which 1 amino acid of SEQ ID NO: 6 is substituted by another amino acid. In one embodiment, in such an ankyrin repeat domain said first ankyrin repeat module comprises the amino acid sequence of SEQ ID NO: 5, and said second ankyrin repeat module comprises the amino acid sequence of SEQ ID NO: 6.

In a preferred embodiment, said first ankyrin repeat module comprises an amino acid sequence selected from the group consisting of (1) SEQ ID NO: 5 and (2) sequences in which up to 3, or up to 2, or up to 1 amino acids in SEQ ID NO: 5 are substituted by other amino acids, and said second ankyrin repeat module comprises an amino acid sequence selected from the group consisting of (1) SEQ ID NO: 6 and (2) sequences in which up to 3, or up to 2, or up to 1 amino acids of SEQ ID NO: 6 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 5 and said second ankyrin repeat module comprises the amino acid sequence of SEQ ID NO: 6, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 7 and (2) sequences in which up to 9, or up to 8, or up to 7, or up to 6, or up to 5, or up to 4, or up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 6 and (2) sequences in which up to 9, or up to 8, or up to 7, or up to 6, or up to 5, or up to 4, or up to 3, or up to 2, or up to 1 amino acids of SEQ ID NO: 6 are substituted by other amino acids.

Thus, in one embodiment, in such an ankyrin repeat domain said first ankyrin repeat module comprises an amino acid sequence selected from the group consisting of (1) SEQ ID NO: 7 and (2) sequences in which up to 9 amino acids in SEQ ID NO: 7 are substituted by other amino acids, and said second ankyrin repeat module comprises an amino acid sequence selected from the group consisting of (1) SEQ ID NO: 6 and (2) sequences in which up to 9 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 6 amino acids in SEQ ID NO: 7 are substituted by other amino acids, and said second ankyrin repeat module comprises an amino acid sequence selected from the group consisting of (1) SEQ ID NO: 6 and (2) sequences in which up to 6 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 5 amino acids in SEQ ID NO: 7 are substituted by other amino acids, and said second ankyrin repeat module comprises an amino acid sequence selected from the group consisting of (1) SEQ ID NO: 6 and (2) sequences in which up to 5 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 4 amino acids in SEQ ID NO: 7 are substituted by other amino acids, and said second ankyrin repeat module comprises an amino acid sequence selected from the group consisting of (1) SEQ ID NO: 6 and (2) sequences in which up to 4 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 3 amino acids in SEQ ID NO: 7 are substituted by other amino acids, and said second ankyrin repeat module comprises an amino acid sequence selected from the group consisting of (1) SEQ ID NO: 6 and (2) sequences in which up to 3 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 2 amino acids in SEQ ID NO: 7 are substituted by other amino acids, and said second ankyrin repeat module comprises an amino acid sequence selected from the group consisting of (1) SEQ ID NO: 6 and (2) sequences in which up to 2 amino acids of SEQ ID NO: 6 are substituted by other amino acids. In one embodiment, in such an ankyrin repeat domain said first ankyrin repeat module comprises an amino acid sequence selected from the group consisting of (1) SEQ ID NO: 7 and (2) sequences in which 1 amino acid in SEQ ID NO: 7 is substituted by another amino acid, and said second ankyrin repeat module comprises an amino acid sequence selected from the group consisting of (1) SEQ ID NO: 6 and (2) sequences in which 1 amino acid of SEQ ID NO: 6 is substituted by another amino acid. In one embodiment, in such an ankyrin repeat domain said first ankyrin repeat module comprises the amino acid sequence of SEQ ID NO: 7, and said second ankyrin repeat module comprises the amino acid sequence of SEQ ID NO: 6.

In one preferred embodiment, said first ankyrin repeat module comprises an amino acid sequence selected from the group consisting of (1) SEQ ID NO: 7 and (2) sequences in which up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 6 and (2) sequences in which up to 3, or up to 2, or up to 1 amino acids of SEQ ID NO: 6 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 7 and said second ankyrin repeat module comprises the amino acid sequence of SEQ ID NO: 6, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 7 and (2) sequences in which up to 9, or up to 8, or up to 7, or up to 6, or up to 5, or up to 4, or up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 8 and (2) sequences in which up to 9, or up to 8, or up to 7, or up to 6, or up to 5, or up to 4, or up to 3, or up to 2, or up to 1 amino acids of SEQ ID NO: 8 are substituted by other amino acids.

Thus, in one embodiment, in such an ankyrin repeat domain said first ankyrin repeat module comprises an amino acid sequence selected from the group consisting of (1) SEQ ID NO: 7 and (2) sequences in which up to 9 amino acids in SEQ ID NO: 7 are substituted by other amino acids, and said second ankyrin repeat module comprises an amino acid sequence selected from the group consisting of (1) SEQ ID NO: 8 and (2) sequences in which up to 9 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 6 amino acids in SEQ ID NO: 7 are substituted by other amino acids, and said second ankyrin repeat module comprises an amino acid sequence selected from the group consisting of (1) SEQ ID NO: 8 and (2) sequences in which up to 6 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 5 amino acids in SEQ ID NO: 7 are substituted by other amino acids, and said second ankyrin repeat module comprises an amino acid sequence selected from the group consisting of (1) SEQ ID NO: 8 and (2) sequences in which up to 5 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 4 amino acids in SEQ ID NO: 7 are substituted by other amino acids, and said second ankyrin repeat module comprises an amino acid sequence selected from the group consisting of (1) SEQ ID NO: 8 and (2) sequences in which up to 4 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 3 amino acids in SEQ ID NO: 7 are substituted by other amino acids, and said second ankyrin repeat module comprises an amino acid sequence selected from the group consisting of (1) SEQ ID NO: 8 and (2) sequences in which up to 3 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 2 amino acids in SEQ ID NO: 7 are substituted by other amino acids, and said second ankyrin repeat module comprises an amino acid sequence selected from the group consisting of (1) SEQ ID NO: 8 and (2) sequences in which up to 2 amino acids of SEQ ID NO: 8 are substituted by other amino acids. In one embodiment, in such an ankyrin repeat domain said first ankyrin repeat module comprises an amino acid sequence selected from the group consisting of (1) SEQ ID NO: 7 and (2) sequences in which 1 amino acid in SEQ ID NO: 7 is substituted by another amino acid, and said second ankyrin repeat module comprises an amino acid sequence selected from the group consisting of (1) SEQ ID NO: 8 and (2) sequences in which 1 amino acid of SEQ ID NO: 8 is substituted by another amino acid. In one embodiment, in such an ankyrin repeat domain said first ankyrin repeat module comprises the amino acid sequence of SEQ ID NO: 7, and said second ankyrin repeat module comprises the amino acid sequence of SEQ ID NO: 8.

In one preferred embodiment, said first ankyrin repeat module comprises an amino acid sequence selected from the group consisting of (1) SEQ ID NO: 7 and (2) sequences in which up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 8 and (2) sequences in which up to 3, or up to 2, or up to 1 amino acids of SEQ ID NO: 8 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 7 and said second ankyrin repeat module comprises the amino acid sequence of SEQ ID NO: 8, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 7 and (2) sequences in which up to 9, or up to 8, or up to 7, or up to 6, or up to 5, or up to 4, or up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 9 and (2) sequences in which up to 9, or up to 8, or up to 7, or up to 6, or up to 5, or up to 4, or up to 3, or up to 2, or up to 1 amino acids of SEQ ID NO: 9 are substituted by other amino acids.

Thus, in one embodiment, in such an ankyrin repeat domain said first ankyrin repeat module comprises an amino acid sequence selected from the group consisting of (1) SEQ ID NO: 7 and (2) sequences in which up to 9 amino acids in SEQ ID NO: 7 are substituted by other amino acids, and said second ankyrin repeat module comprises an amino acid sequence selected from the group consisting of (1) SEQ ID NO: 9 and (2) sequences in which up to 9 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 6 amino acids in SEQ ID NO: 7 are substituted by other amino acids, and said second ankyrin repeat module comprises an amino acid sequence selected from the group consisting of (1) SEQ ID NO: 9 and (2) sequences in which up to 6 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 5 amino acids in SEQ ID NO: 7 are substituted by other amino acids, and said second ankyrin repeat module comprises an amino acid sequence selected from the group consisting of (1) SEQ ID NO: 9 and (2) sequences in which up to 5 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 4 amino acids in SEQ ID NO: 7 are substituted by other amino acids, and said second ankyrin repeat module comprises an amino acid sequence selected from the group consisting of (1) SEQ ID NO: 9 and (2) sequences in which up to 4 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 3 amino acids in SEQ ID NO: 7 are substituted by other amino acids, and said second ankyrin repeat module comprises an amino acid sequence selected from the group consisting of (1) SEQ ID NO: 9 and (2) sequences in which up to 3 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which up to 2 amino acids in SEQ ID NO: 7 are substituted by other amino acids, and said second ankyrin repeat module comprises an amino acid sequence selected from the group consisting of (1) SEQ ID NO: 9 and (2) sequences in which up to 2 amino acids of SEQ ID NO: 9 are substituted by other amino acids. In one embodiment, in such an ankyrin repeat domain said first ankyrin repeat module comprises an amino acid sequence selected from the group consisting of (1) SEQ ID NO: 7 and (2) sequences in which 1 amino acid in SEQ ID NO: 7 is substituted by another amino acid, and said second ankyrin repeat module comprises an amino acid sequence selected from the group consisting of (1) SEQ ID NO: 9 and (2) sequences in which 1 amino acid of SEQ ID NO: 9 is substituted by another amino acid In one embodiment, in such an ankyrin repeat domain said first ankyrin repeat module comprises the amino acid sequence of SEQ ID NO: 7, and said second ankyrin repeat module comprises the amino acid sequence of SEQ ID NO: 9.

In a preferred embodiment, said first ankyrin repeat module comprises an amino acid sequence selected from the group consisting of (1) SEQ ID NO: 7 and (2) sequences in which up to 3, or up to 2, or up to 1 amino acids in SEQ ID NO: 7 are substituted by other amino acids, and said second ankyrin repeat module comprises an amino acid sequence selected from the group consisting of (1) SEQ ID NO: 9 and (2) sequences in which up to 3, or up to 2, or up to 1 amino acids of SEQ ID NO: 9 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 7 and said second ankyrin repeat module comprises the amino acid sequence of SEQ ID NO: 9, wherein said first ankyrin repeat module is located N-terminally of said second ankyrin repeat module within said ankyrin repeat domain.

In one preferred embodiment, all of said amino acid substitutions of said ankyrin repeat module(s) as described and referred to herein occur in framework positions of said ankyrin repeat module(s), wherein typically the overall structure of the module(s) is not affected by the substitutions. Such an embodiment of substitution in framework positions shall apply to all embodiments irrespective of whether such substitution is explicitly described.

In one preferred embodiment, all of said amino acid substitutions of said ankyrin repeat module(s) as described and referred to herein occur in framework positions and in positions other than positions 3, 4, 6, 14 and 15, preferably other than positions 2, 3, 4, 5, 6, 14 and 15, of said ankyrin repeat module(s) of SEQ ID NOs: 5 to 9, wherein typically the overall structure of the module(s) is not affected by the substitutions.

In another aspect, the invention relates to a recombinant binding protein comprising an ankyrin repeat domain, wherein said ankyrin repeat domain has binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N.

Thus, in one embodiment, said ankyrin repeat domain comprises an amino acid sequence with at least 80% amino acid sequence identity with any one of SEQ ID NOs: 1 to 4. In one embodiment, said ankyrin repeat domain comprises an amino acid sequence with at least 90% amino acid sequence identity with any one of SEQ ID NOs: 1 to 4. In another embodiment, said ankyrin repeat domain comprises an amino acid sequence with at least 93% amino acid sequence identity with any one of SEQ ID NOs: 1 to 4; and in a further embodiment, said ankyrin repeat domain comprises an amino acid sequence with at least 95% amino acid sequence identity with any one of SEQ ID NOs: 1 to 4. In one embodiment, said ankyrin repeat domain comprises an amino acid sequence with at least 98% amino acid sequence identity with any one of SEQ ID NOs: 1 to 4; and in one embodiment, said ankyrin repeat domain comprises the amino acid sequence of any one of SEQ ID NOs: 1 to 4. Thus, in one embodiment, said recombinant binding protein comprises an ankyrin repeat domain with binding specificity for CD3, wherein said ankyrin repeat domain comprises an amino acid sequence selected from SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, and wherein A at the second last position of SEQ ID NO: 1 is optionally substituted by L, and/or A at the last position of SEQ ID NO: 1 is optionally substituted by N. Thus, in one embodiment, said ankyrin repeat domain comprises an amino acid sequence with at least 80% amino acid sequence identity with SEQ ID NO: 1. In one embodiment, said ankyrin repeat domain comprises an amino acid sequence with at least 90% amino acid sequence identity with SEQ ID NO: 1. In another embodiment, said ankyrin repeat domain comprises an amino acid sequence with at least 93% amino acid sequence identity with SEQ ID NO: 1; and in a further embodiment, said ankyrin repeat domain comprises an amino acid sequence with at least 95% amino acid sequence identity with SEQ ID NO: 1. In one embodiment, said ankyrin repeat domain comprises an amino acid sequence with at least 98% amino acid sequence identity with SEQ ID NO: 1; and in one embodiment, said ankyrin repeat domain comprises the amino acid sequence of SEQ ID NO: 1. Thus, in one embodiment, said recombinant binding protein comprises an ankyrin repeat domain having binding specificity for CD3, wherein said ankyrin repeat domain comprises the amino acid sequence of SEQ ID NO: 1.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, and wherein A at the second last position of SEQ ID NO: 2 is optionally substituted by L, and/or A at the last position of SEQ ID NO: 2 is optionally substituted by N. Thus, in one embodiment, said ankyrin repeat domain comprises an amino acid sequence with at least 80% amino acid sequence identity with SEQ ID NO: 2. In one embodiment, said ankyrin repeat domain comprises an amino acid sequence with at least 90% amino acid sequence identity with SEQ ID NO: 2. In another embodiment, said ankyrin repeat domain comprises an amino acid sequence with at least 93% amino acid sequence identity with SEQ ID NO: 2; and in a further embodiment, said ankyrin repeat domain comprises an amino acid sequence with at least 95% amino acid sequence identity with SEQ ID NO: 2. In one embodiment, said ankyrin repeat domain comprises an amino acid sequence with at least 98% amino acid sequence identity with SEQ ID NO: 2; and in one embodiment, said ankyrin repeat domain comprises the amino acid sequence of SEQ ID NO: 2. Thus, in one embodiment, said recombinant binding protein comprises an ankyrin repeat domain having binding specificity for CD3, wherein said ankyrin repeat domain comprises the amino acid sequence of SEQ ID NO: 2.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, and wherein A at the second last position of SEQ ID NO: 3 is optionally substituted by L, and/or A at the last position of SEQ ID NO: 3 is optionally substituted by N. Thus, in one embodiment, said ankyrin repeat domain comprises an amino acid sequence with at least 80% amino acid sequence identity with SEQ ID NO: 3. In one embodiment, said ankyrin repeat domain comprises an amino acid sequence with at least 90% amino acid sequence identity with SEQ ID NO: 3. In another embodiment, said ankyrin repeat domain comprises an amino acid sequence with at least 93% amino acid sequence identity with SEQ ID NO: 3; and in a further embodiment, said ankyrin repeat domain comprises an amino acid sequence with at least 95% amino acid sequence identity with SEQ ID NO: 3. In one embodiment, said ankyrin repeat domain comprises an amino acid sequence with at least 98% amino acid sequence identity with SEQ ID NO: 3; and in one embodiment, said ankyrin repeat domain comprises the amino acid sequence of SEQ ID NO: 3. Thus, in one embodiment, said recombinant binding protein comprises an ankyrin repeat domain having binding specificity for CD3, wherein said ankyrin repeat domain comprises the amino acid sequence of SEQ ID NO: 3.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, and wherein A at the second last position of SEQ ID NO: 4 is optionally substituted by L, and/or A at the last position of SEQ ID NO: 4 is optionally substituted by N. Thus, in one embodiment, said ankyrin repeat domain comprises an amino acid sequence with at least 80% amino acid sequence identity with SEQ ID NO: 4. In one embodiment, said ankyrin repeat domain comprises an amino acid sequence with at least 90% amino acid sequence identity with SEQ ID NO: 4. In another embodiment, said ankyrin repeat domain comprises an amino acid sequence with at least 93% amino acid sequence identity with SEQ ID NO: 4; and in a further embodiment, said ankyrin repeat domain comprises an amino acid sequence with at least 95% amino acid sequence identity with SEQ ID NO: 4. In one embodiment, said ankyrin repeat domain comprises an amino acid sequence with at least 98% amino acid sequence identity with SEQ ID NO: 4; and in one embodiment, said ankyrin repeat domain comprises the amino acid sequence of SEQ ID NO: 4. Thus, in one embodiment, said recombinant binding protein comprises an ankyrin repeat domain having binding specificity for CD3, wherein said ankyrin repeat domain comprises the amino acid sequence of SEQ ID NO: 4.

In one embodiment, said recombinant binding protein comprises an ankyrin repeat domain with binding specificity for CD3, wherein the potential interaction residues in said ankyrin repeat domain are identical to the corresponding positions in any one of the ankyrin repeat domains of SEQ ID NOs: 1 to 4.

In one embodiment, said recombinant binding protein comprises an ankyrin repeat domain with binding specificity for CD3, wherein said binding protein binds human CD3 on T cells with an EC₅₀ of less than 900, 700, 500, 400, 300, 200, 150, 100, 70, 60, 50, 40, 30, 20, 15, or 10 nM. Thus, in one embodiment, said binding protein binds human CD3 on T cells with an EC₅₀ of less than 900 nM; in another embodiment, said binding protein binds human CD3 on T cells with an EC₅₀ of less than 700 nM; in another embodiment, said binding protein binds human CD3 on T cells with an EC₅₀ of less than 500 nM; in another embodiment, said binding protein binds human CD3 on T cells with an EC₅₀ of less than 400 nM; in another embodiment, said binding protein binds human CD3 on T cells with an EC₅₀ of less than 300 nM; in another embodiment, said binding protein binds human CD3 on T cells with an EC₅₀ of less than 200 nM; in another embodiment, said binding protein binds human CD3 on T cells with an EC₅₀ of less than 100 nM; in another embodiment, said binding protein binds human CD3 on T cells with an EC₅₀ of less than 70 nM; in another embodiment, said binding protein binds human CD3 on T cells with an EC₅₀ of less than 60 nM; in another embodiment, said binding protein binds human CD3 on T cells with an EC₅₀ of less than 50 nM; in another embodiment, said binding protein binds human CD3 on T cells with an EC₅₀ of less than 40 nM; in another embodiment, said binding protein binds to T cells with an EC₅₀ of less than 30 nM; in another embodiment, said binding protein binds to T cells with an EC₅₀ of less than 20 nM; in another embodiment, said binding protein binds to T cells with an EC₅₀ of less than 15 nM; in a further embodiment, said binding protein binds to T cells with an EC₅₀ of less than 10 nM.

In one embodiment, said recombinant binding protein comprises an ankyrin repeat domain with binding specificity for CD3, wherein said binding protein binds human CD3 on T cells with an EC₅₀ ranging from 2 to 900 nM. In another embodiment, said recombinant binding protein comprises an ankyrin repeat domain with binding specificity for CD3, wherein said binding protein binds human CD3 on T cells with an EC so ranging from about 5 to about 400 nM. In a further embodiment, said recombinant binding protein comprises an ankyrin repeat domain with binding specificity for CD3, wherein said binding protein binds human CD3 on T cells with an EC₅₀ ranging from about 250 to about 400 nM; in another embodiment said binding protein binds human CD3 on T cell with an EC₅₀ ranging from about 50 to about 100 nM; in a further embodiment said binding protein binds human CD3 on T cells with an EC₅₀ ranging from about 5 to about 15 nM.

A typical and preferred determination of CD3 binding on T cells (EC₅₀) of the inventive recombinant binding proteins with binding specificity for CD3 by using Mirrorball laser scanning imaging cytometry is described in Example 5 (with primary human T cells). Thus, in one embodiment said CD3 binding (EC₅₀) of the inventive recombinant binding proteins is determined on primary human T cells by Mirrorball laser scanning imaging cytometry as described in Example 5.

In one embodiment, said recombinant binding protein comprises an ankyrin repeat domain with binding specificity for CD3, wherein said binding protein binds human CD3 on T cells with an EC₅₀ ranging from 2 to 900 nM from 5 to 400 nM, and wherein said binding protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N. Thus, in one embodiment, said binding protein binds human CD3 on T cells with an EC₅₀ ranging from 2 to 900 nM, and said ankyrin repeat domain comprises an amino acid sequence with at least 80% amino acid sequence identity with any one of SEQ ID NOs: 1 to 4. In one embodiment, said binding protein binds human CD3 on T cells with an EC₅₀ ranging from 2 to 900 nM, and said ankyrin repeat domain comprises an amino acid sequence with at least 90% amino acid sequence identity with any one of SEQ ID NOs: 1 to 4. In another embodiment, said binding protein binds human CD3 on T cells with an EC₅₀ ranging from 2 to 900 nM, and said ankyrin repeat domain comprises an amino acid sequence with at least 93% amino acid sequence identity with any one of SEQ ID NOs: 1 to 4; and in a further embodiment, said binding protein binds human CD3 on T cells with an EC₅₀ ranging from 2 to 900 nM, and said ankyrin repeat domain comprises an amino acid sequence with at least 95% amino acid sequence identity with any one of SEQ ID NOs: 1 to 4. In one embodiment, said binding protein binds human CD3 on T cells with an EC₅₀ ranging from 2 to 900 nM, and said ankyrin repeat domain comprises an amino acid sequence with at least 98% amino acid sequence identity with any one of SEQ ID NOs: 1 to 4; and in one embodiment, said binding protein binds human CD3 on T cells with an EC₅₀ ranging from 2 to 900 nM, and said ankyrin repeat domain comprises the amino acid sequence of any one of SEQ ID NOs: 1 to 4. Thus, in one embodiment, said recombinant binding protein comprises an ankyrin repeat domain with binding specificity for CD3, wherein said binding protein binds human CD3 on T cells with an EC₅₀ ranging from 2 to 900 nM, and wherein said ankyrin repeat domain comprises the amino acid sequence of any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N.

In one embodiment, said recombinant binding protein comprises an ankyrin repeat domain with binding specificity for CD3, wherein said binding protein binds human CD3 on T cells with an EC₅₀ ranging from about 250 to about 400 nM, and wherein said binding protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NO: 2, and wherein A at the second last position of SEQ ID NO: 2 is optionally substituted by L, and/or A at the last position of SEQ ID NO: 2 is optionally substituted by N. Thus, in one embodiment, said binding protein binds human CD3 on T cells with an EC₅₀ ranging from 250 to 400 nM, and said ankyrin repeat domain comprises an amino acid sequence with at least 80% amino acid sequence identity with SEQ ID NO: 2. In one embodiment, said binding protein binds human CD3 on T cells with an EC₅₀ ranging from 250 to 400 nM, and said ankyrin repeat domain comprises an amino acid sequence with at least 90% amino acid sequence identity with SEQ ID NO: 2. In another embodiment, said binding protein binds human CD3 on T cells with an EC₅₀ ranging from 250 to 400 nM, and said ankyrin repeat domain comprises an amino acid sequence with at least 93% amino acid sequence identity with SEQ ID NO: 2; and in a further embodiment, said binding protein binds human CD3 on T cells with an EC₅₀ ranging from 250 to 400 nM, and said ankyrin repeat domain comprises an amino acid sequence with at least 95% amino acid sequence identity with SEQ ID NO: 2. In one embodiment, said binding protein binds human CD3 on T cells with an EC₅₀ ranging from 250 to 400 nM, and said ankyrin repeat domain comprises an amino acid sequence with at least 98% amino acid sequence identity with SEQ ID NO: 2; and in one embodiment, said binding protein binds human CD3 on T cells with an EC₅₀ ranging from 250 to 400 nM, and said ankyrin repeat domain comprises the amino acid sequence of SEQ ID NO: 2. Thus, in one embodiment, said recombinant binding protein comprises an ankyrin repeat domain with binding specificity for CD3, wherein said binding protein binds human CD3 on T cells with an EC₅₀ ranging from about 250 to about 400 nM, and wherein said ankyrin repeat domain comprises the amino acid sequence of SEQ ID NO: 2, wherein A at the second last position of SEQ ID NO: 2 is optionally substituted by L, and/or A at the last position of SEQ ID NO: 2 is optionally substituted by N.

In one embodiment, said recombinant binding protein comprises an ankyrin repeat domain with binding specificity for CD3, wherein said binding protein binds human CD3 on T cells with an EC₅₀ ranging from about 50 to about 100 nM, and wherein said binding protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NO: 3, and wherein A at the second last position of SEQ ID NO: 3 is optionally substituted by L, and/or A at the last position of SEQ ID NO: 3 is optionally substituted by N. Thus, in one embodiment, said binding protein binds human CD3 on T cells with an EC₅₀ ranging from 50 to 100 nM, and said ankyrin repeat domain comprises an amino acid sequence with at least 80% amino acid sequence identity with SEQ ID NO: 3. In one embodiment, said binding protein binds human CD3 on T cells with an EC₅₀ ranging from 50 to 100 nM, and said ankyrin repeat domain comprises an amino acid sequence with at least 90% amino acid sequence identity with SEQ ID NO: 3. In another embodiment, said binding protein binds human CD3 on T cells with an EC₅₀ ranging from 50 to 100 nM, and said ankyrin repeat domain comprises an amino acid sequence with at least 93% amino acid sequence identity with SEQ ID NO: 3; and in a further embodiment, said binding protein binds human CD3 on T cells with an EC₅₀ ranging from 50 to 100 nM, and said ankyrin repeat domain comprises an amino acid sequence with at least 95% amino acid sequence identity with SEQ ID NO: 3. In one embodiment, said binding protein binds human CD3 on T cells with an EC₅₀ ranging from 50 to 100 nM, and said ankyrin repeat domain comprises an amino acid sequence with at least 98% amino acid sequence identity with SEQ ID NO: 3; and in one embodiment, said binding protein binds human CD3 on T cells with an EC₅₀ ranging from 50 to 100 nM, and said ankyrin repeat domain comprises the amino acid sequence of SEQ ID NO: 3. Thus, in one embodiment, said recombinant binding protein comprises an ankyrin repeat domain with binding specificity for CD3, wherein said binding protein binds human CD3 on T cells with an EC₅₀ ranging from about 50 to about 100 nM, and wherein said ankyrin repeat domain comprises the amino acid sequence of SEQ ID NO: 3, wherein A at the second last position of SEQ ID NO: 3 is optionally substituted by L, and/or A at the last position of SEQ ID NO: 3 is optionally substituted by N.

In one embodiment, said recombinant binding protein comprises an ankyrin repeat domain with binding specificity for CD3, wherein said binding protein binds human CD3 on T cells with an EC 50 ranging from about 5 to about 15 nM, and wherein said binding protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NO: 4, and wherein A at the second last position of SEQ ID NO: 4 is optionally substituted by L, and/or A at the last position of SEQ ID NO: 4 is optionally substituted by N. Thus, in one embodiment, said binding protein binds human CD3 on T cells with an EC₅₀ ranging from 5 to 15 nM, and said ankyrin repeat domain comprises an amino acid sequence with at least 80% amino acid sequence identity with SEQ ID NO: 4. In one embodiment, said binding protein binds human CD3 on T cells with an EC₅₀ ranging from 5 to 15 nM, and said ankyrin repeat domain comprises an amino acid sequence with at least 90% amino acid sequence identity with SEQ ID NO: 4. In another embodiment, said binding protein binds human CD3 on T cells with an EC₅₀ ranging from 5 to 15 nM, and said ankyrin repeat domain comprises an amino acid sequence with at least 93% amino acid sequence identity with SEQ ID NO: 4; and in a further embodiment, said binding protein binds human CD3 on T cells with an EC₅₀ ranging from 5 to 15 nM, and said ankyrin repeat domain comprises an amino acid sequence with at least 95% amino acid sequence identity with SEQ ID NO: 4. In one embodiment, said binding protein binds human CD3 on T cells with an EC₅₀ ranging from 5 to 15 nM, and said ankyrin repeat domain comprises an amino acid sequence with at least 98% amino acid sequence identity with SEQ ID NO: 4; and in one embodiment, said binding protein binds human CD3 on T cells with an EC₅₀ ranging from 5 to 15 nM, and said ankyrin repeat domain comprises the amino acid sequence of SEQ ID NO: 4. Thus, in one embodiment, said recombinant binding protein comprises an ankyrin repeat domain with binding specificity for CD3, wherein said binding protein binds human CD3 on T cells with an EC₅₀ ranging from about 5 to about 15 nM, and wherein said ankyrin repeat domain comprises the amino acid sequence of SEQ ID NO: 4, wherein A at the second last position of SEQ ID NO: 4 is optionally substituted by L, and/or A at the last position of SEQ ID NO: 4 is optionally substituted by N.

In one embodiment, the invention relates to a recombinant binding protein comprising an ankyrin repeat domain with binding specificity for CD3, wherein said binding protein binds to human CD3 in PBS with a dissociation constant (K_{D}) of or below about 10⁻⁶ M, or of or below about 10⁻⁷ M, or of or below about 5×10⁻⁸ M, or of or below about 2×10⁻⁸ M, or of or below about 10⁻⁸ M. Thus, in one embodiment, said binding protein binds human CD3 in PBS with a dissociation constant (K_{D}) of or below about 10⁻⁶ M. In another embodiment, said binding protein binds human CD3 in PBS with a dissociation constant (K_{D}) of or below below about 10⁻⁷ M. In another embodiment, said binding protein binds human CD3 in PBS with a dissociation constant (K_{D}) of or below about 5×10⁻⁸ M; and in a further embodiment, said binding binds human CD3 in PBS with a dissociation constant (K_{D}) of or below about 2×10⁻⁸ M. In another embodiment, said binding protein binds human CD3 in PBS with a dissociation constant (K_{D}) of or below about 10⁻⁸ M.

In one embodiment, said recombinant binding protein comprises an ankyrin repeat domain with binding specificity for CD3, wherein said binding protein binds human CD3 in PBS with a dissociation constant (K_{D}) of or below 10⁻⁶ M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N. In another embodiment, said recombinant binding protein comprises an ankyrin repeat domain with binding specificity for CD3, wherein said binding protein binds human CD3 in PBS with a dissociation constant (K_{D}) of or below 10⁻⁷ M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N. Thus, in one embodiment, said binding protein binds human CD3 in PBS with a dissociation constant (K_{D}) below 10⁻⁷ M, and said ankyrin repeat domain comprises an amino acid sequence with at least 80% amino acid sequence identity with any one of SEQ ID NOs: 1 to 4. In one embodiment, said binding protein binds human CD3 in PBS with a dissociation constant (K_{D}) below 10⁻⁷ M, and said ankyrin repeat domain comprises an amino acid sequence with at least 90% amino acid sequence identity with any one of SEQ ID NOs: 1 to 4. In another embodiment, said binding protein binds human CD3 in PBS with a dissociation constant (K_{D}) below 10⁻⁷ M, and said ankyrin repeat domain comprises an amino acid sequence with at least 93% amino acid sequence identity with any one of SEQ ID NOs: 1 to 4; and in a further embodiment, said binding protein binds human CD3 in PBS with a dissociation constant (K_{D}) below 10⁻⁷ M, and said ankyrin repeat domain comprises an amino acid sequence with at least 95% amino acid sequence identity with any one of SEQ ID NOs: 1 to 4. In one embodiment, said binding protein binds human CD3 in PBS with a dissociation constant (K_{D}) below 10⁻⁷ M, and said ankyrin repeat domain comprises an amino acid sequence with at least 98% amino acid sequence identity with any one of SEQ ID NOs: 1 to 4; and in one embodiment, said binding protein binds human CD3 in PBS with a dissociation constant (K_{D}) below 10⁻⁷ M, and said ankyrin repeat domain comprises the amino acid sequence of any one of SEQ ID NOs: 1 to 4. Thus, in one embodiment, said recombinant binding protein comprises an ankyrin repeat domain with binding specificity for CD3, wherein said binding protein binds human CD3 in PBS with a dissociation constant (K_{D}) of or below about 10⁻⁷ M, and wherein said ankyrin repeat domain comprises the amino acid sequence of any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N.

In one embodiment, said recombinant binding protein comprises an ankyrin repeat domain with binding specificity for CD3, wherein said binding protein binds human CD3 in PBS with a dissociation constant (K_{D}) of or below about 5×10⁻⁸ M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 4, wherein A at the second last position of SEQ ID NOs:1 to 4 is optionally substituted by L, and/or A at the last position of SEQ ID NOs: 1 to 4 is optionally substituted by N. Thus, in one embodiment, said binding protein binds human CD3 in PBS with a dissociation constant (K_{D}) of or below about 5×10⁻⁸ M, and said ankyrin repeat domain comprises an amino acid sequence with at least 80% amino acid sequence identity with any one of SEQ ID NOs: 1 to 4. In one embodiment, said binding protein binds human CD3 in PBS with a dissociation constant (K_{D}) below 5×10⁻⁸ M, and said ankyrin repeat domain comprises an amino acid sequence with at least 90% amino acid sequence identity with any one of SEQ ID NOs: 1 to 4. In another embodiment, said binding protein binds human CD3 in PBS with a dissociation constant (K_{D}) below 5×10⁻⁸ M, and said ankyrin repeat domain comprises an amino acid sequence with at least 93% amino acid sequence identity with any one of SEQ ID NOs: 1 to 4; and in a further embodiment, said binding protein binds human CD3 in PBS with a dissociation constant (K_{D}) below 5×10⁻⁸ M, and said ankyrin repeat domain comprises an amino acid sequence with at least 95% amino acid sequence identity with any one of SEQ ID NOs: 1 to 4. In one embodiment, said binding protein binds human CD3 in PBS with a dissociation constant (K_{D}) below 5×10⁻⁸ M, and said ankyrin repeat domain comprises an amino acid sequence with at least 98% amino acid sequence identity with any one of SEQ ID NOs: 1 to 4; and in one embodiment, said binding protein binds human CD3 in PBS with a dissociation constant (K_{D}) below 5×10⁻⁸ M, and said ankyrin repeat domain comprises the amino acid sequence of any one of SEQ ID NOs: 1 to 4. Thus, in one embodiment, said recombinant binding protein comprises an ankyrin repeat domain with binding specificity for CD3, wherein said binding protein binds human CD3 in PBS with a dissociation constant (K_{D}) of or below about 5×10⁻⁸ M, and wherein said ankyrin repeat domain comprises the amino acid sequence of any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N.

In one embodiment, said recombinant binding protein comprises an ankyrin repeat domain having binding specificity for CD3, wherein said binding protein binds human CD3 in PBS with a dissociation constant (K_{D}) of or below about 2×10⁻⁸ M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 3 or 4, wherein A at the second last position of SEQ ID NOs:1 to 4 is optionally substituted by L, and/or A at the last position of SEQ ID NO: 3 or 4 is optionally substituted by N. Thus, in one embodiment, said binding protein binds human CD3 in PBS with a dissociation constant (K_{D}) of or below about 2×10⁻⁸ M, and said ankyrin repeat domain comprises an amino acid sequence with at least 80% amino acid sequence identity with SEQ ID NO: 3 or 4. In one embodiment, said binding protein binds human CD3 in PBS with a dissociation constant (K_{D}) below 2×10⁻⁸ M, and said ankyrin repeat domain comprises an amino acid sequence with at least 90% amino acid sequence identity with SEQ ID NO: 3 or 4. In another embodiment, said binding protein binds human CD3 in PBS with a dissociation constant (K_{D}) below 2×10⁻⁸ M, and said ankyrin repeat domain comprises an amino acid sequence with at least 93% amino acid sequence identity with SEQ ID NO: 3 or 4; and in a further embodiment, said binding protein binds human CD3 in PBS with a dissociation constant (K_{D}) below 2×10⁻⁸ M, and said ankyrin repeat domain comprises an amino acid sequence with at least 95% amino acid sequence identity with SEQ ID NO: 3 or 4. In one embodiment, said binding protein binds human CD3 in PBS with a dissociation constant (K_{D}) below 2×10⁻⁸ M, and said ankyrin repeat domain comprises an amino acid sequence with at least 98% amino acid sequence identity with SEQ ID NO: 3 or 4; and in one embodiment, said binding protein binds human CD3 in PBS with a dissociation constant (K_{D}) below 2 ×10⁻⁸ M, and said ankyrin repeat domain comprises the amino acid sequence of SEQ ID NO: 3 or 4. Thus, in one embodiment, said recombinant binding protein comprises an ankyrin repeat domain with binding specificity for CD3, wherein said binding protein binds human CD3 in PBS with a dissociation constant (K_{D}) of or below about 2×10⁻⁸ M, and wherein said ankyrin repeat domain comprises the amino acid sequence of SEQ ID NO: 3 or 4, wherein A at the second last position of SEQ ID NO: 3 or 4 is optionally substituted by L, and/or A at the last position of SEQ ID NO: 3 or 4 is optionally substituted by N.

In one embodiment, said recombinant binding protein comprises an ankyrin repeat domain having binding specificity for CD3, wherein said binding protein binds human CD3 in PBS with a dissociation constant (K_{D}) of or below about 10⁻⁸ M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 4, and wherein A at the second last position of SEQ ID NO: 4 is optionally substituted by L, and/or A at the last position of SEQ ID NO: 4 is optionally substituted by N. Thus, in one embodiment, said binding protein binds human CD3 in PBS with a dissociation constant (K_{D}) of or below about 10⁻⁸ M, and said ankyrin repeat domain comprises an amino acid sequence with at least 80% amino acid sequence identity with the SEQ ID NO: 4. In one embodiment, said binding protein binds human CD3 in PBS with a dissociation constant (K_{D}) below 10⁻⁸ M, and said ankyrin repeat domain comprises an amino acid sequence with at least 90% amino acid sequence identity with the SEQ ID NO: 4. In another embodiment, said binding protein binds human CD3 in PBS with a dissociation constant (K_{D}) below 10⁻⁸ M, and said ankyrin repeat domain comprises an amino acid sequence with at least 93% amino acid sequence identity with SEQ ID NO: 4; and in a further embodiment, said binding protein binds human CD3 in PBS with a dissociation constant (K_{D}) below 10⁻⁸ M, and said ankyrin repeat domain comprises an amino acid sequence with at least 95% amino acid sequence identity with f SEQ ID NO: 4. In one embodiment, said binding protein binds human CD3 in PBS with a dissociation constant (K_{D}) below 10⁻⁸ M, and said ankyrin repeat domain comprises an amino acid sequence with at least 98% amino acid sequence identity with SEQ ID NO: 4; and in one embodiment, said binding protein binds human CD3 in PBS with a dissociation constant (K_{D}) below 10⁻⁸ M, and said ankyrin repeat domain comprises the amino acid sequence of SEQ ID NO: 4. Thus, in one embodiment, said recombinant binding protein comprises an ankyrin repeat domain with binding specificity for CD3, wherein said binding protein binds human CD3 in PBS with a dissociation constant (K_{D}) of or below about 10⁻⁸ M, and wherein said ankyrin repeat domain comprises the amino acid sequence of SEQ ID NO: 4, and wherein A at the second last position of SEQ ID NO: 4 is optionally substituted by L, and/or A at the last position of SEQ ID NO: 4 is optionally substituted.

A typical and preferred determination of dissociation constants (K_{D}) of the inventive recombinant binding proteins with binding specificity for CD3 by Surface Plasmon Resonance (SPR) analysis is described in Example 4. Thus, in one embodiment said binding specificity for CD3 of the inventive recombinant binding proteins is determined in PBS by Surface Plasmon Resonance (SPR). In one embodiment said binding specificity for CD3 of the inventive recombinant binding proteins is determined in PBS by Surface Plasmon Resonance (SPR) as described in Example 4.

In one embodiment, the invention relates to a recombinant binding protein comprising an ankyrin repeat domain with binding specificity for CD3, wherein said ankyrin repeat domain has a melting temperature (Tₘ) higher than 65° C., higher than 68° C., higher than 70° C., higher than 72° C., higher than 75° C., higher than 78° C., higher than 80° C., of about 75° C., of about 80° C., of about 82° C., of about 85° C., of between 65° C. and 95° C., of between 70° C. and 90° C., or of between 72° C. and 88° C. Thus, in one embodiment, said ankyrin repeat domain has a melting temperature (Tₘ) higher than 65° C. In another embodiment, said ankyrin repeat domain has a melting temperature (Tₘ) higher than 70° C. In another embodiment, said ankyrin repeat domain has a melting temperature (Tₘ) higher than 75° C. In another embodiment, said ankyrin repeat domain has a melting temperature (Tₘ) higher than 80° C. In another embodiment, said ankyrin repeat domain has a melting temperature (Tₘ) of between 65° C. and 95° C. In another embodiment, said ankyrin repeat domain has a melting temperature (Tₘ) of between 72° C. and 88° C. In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N; and wherein said ankyrin repeat domain has a melting temperature (Tₘ) higher than 65° C. In one embodiment, said ankyrin repeat domain has a melting temperature (Tₘ) higher than 68° C. In another embodiment, said ankyrin repeat domain has a melting temperature (Tₘ) higher than 70° C. In another embodiment, said ankyrin repeat domain has a melting temperature (Tₘ) higher than 72° C. In another embodiment, said ankyrin repeat domain has a melting temperature (Tₘ) higher than 75° C. In another embodiment, said ankyrin repeat domain has a melting temperature (Tₘ) higher than 78° C. In a further embodiment, said ankyrin repeat domain has a melting temperature (Tₘ) higher than 80° C. In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein A at the second last position of SEQ ID NO: 1 is optionally substituted by L, and/or A at the last position of SEQ ID NO: 1 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 95° C. In one embodiment, said ankyrin repeat domain has a melting temperature (Tₘ) of about 85° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, wherein A at the second last position of SEQ ID NO: 2 is optionally substituted by L, and/or A at the last position of SEQ ID NO: 2 is optionally substituted by N; and wherein said ankyrin repeat domain has a melting temperature (Tₘ) higher than 72° C.

In one embodiment, said ankyrin repeat domain has a melting temperature (Tₘ) higher than 75° C. In one embodiment, said ankyrin repeat domain has a melting temperature (Tₘ) higher than 77° C. In one embodiment, said ankyrin repeat domain has a melting temperature (Tₘ) of between 72° C. and 92° C. In one embodiment, said ankyrin repeat domain has a melting temperature (Tₘ) of about 82° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, wherein A at the second last position of SEQ ID NO: 3 is optionally substituted by L, and/or A at the last position of SEQ ID NO: 3 is optionally substituted by N; and wherein said ankyrin repeat domain has a melting temperature (Tₘ) higher than 65° C. In one embodiment, said ankyrin repeat domain has a melting temperature (Tₘ) higher than 68° C. In one embodiment, said ankyrin repeat domain has a melting temperature (Tₘ) higher than 70° C. In one embodiment, said ankyrin repeat domain has a melting temperature (Tₘ) of between 65° C. and 85° C. In one embodiment, said ankyrin repeat domain has a melting temperature (Tₘ) of about 75° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, wherein A at the second last position of SEQ ID NO: 4 is optionally substituted by L, and/or A at the last position of SEQ ID NO: 4 is optionally substituted by N; and wherein said ankyrin repeat domain has a melting temperature (Tₘ) higher than 73° C. In one embodiment, said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) of between 73° C. and 93° C. In one embodiment, said ankyrin repeat domain has a melting temperature (Tₘ) of about 83° C.

A typical and preferred determination of the melting temperature (Tₘ) of the inventive recombinant binding proteins or ankyrin repeat domains with binding specificity for CD3 by Circular Dichroism (CD) spectroscopy is described in Example 2. Thus, in one embodiment said melting temperature (Tₘ) of the inventive recombinant binding proteins or ankyrin repeat domains is determined in TBS pH 8.0 (50 mM Tris, 500 mM NaCl) by Circular Dichroism (CD) spectroscopy. In one embodiment said melting temperature (Tₘ) of the inventive recombinant binding proteins or ankyrin repeat domains is determined in TBS pH 8.0 (50 mM Tris, 500 mM NaCl) by Circular Dichroism (CD) spectroscopy as described in Example 2.

In one embodiment, the invention relates to a recombinant binding protein comprising an ankyrin repeat domain with binding specificity for CD3, wherein said ankyrin repeat domain has an aggregation onset temperature (T_{agg}) higher than 65° C., higher than 68° C., higher than 70° C., higher than 72° C., higher than 75° C., higher than 78° C., higher than 80° C., higher than 82° C., higher than 85° C., of between 75° C. and 100° C. of between 70° C. and 95° C., or of between 75° C. and 95° C. Thus, in one embodiment, said ankyrin repeat domain has an aggregation onset temperature (T_{agg}) higher than 65° C. In another embodiment, said ankyrin repeat domain has an aggregation onset temperature (T_{agg}) higher than 70° C. In another embodiment, said ankyrin repeat domain has an aggregation onset temperature (T_{agg}) higher than 75° C. In another embodiment, said ankyrin repeat domain has an aggregation onset temperature (T_{agg}) higher than 80° C. In another embodiment, said ankyrin repeat domain has an aggregation onset temperature (T_{agg}) of between 75° C. and 100° C. In another embodiment, said ankyrin repeat domain has an aggregation onset temperature (T_{agg}) of between 70° C. and 95° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1 to 4, wherein A at the second last position of SEQ ID NOs: 1 to 4 is optionally substituted by L, and/or A at the last position of SEQ ID NOs: 1 to 4 is optionally substituted by N; and wherein said ankyrin repeat domain has an aggregation onset temperature (T_{agg}) higher than 65° C. In one embodiment, said ankyrin repeat domain has an aggregation onset temperature (T_{agg}) higher than 68° C. In another embodiment, said ankyrin repeat domain has an aggregation onset temperature (T_{agg}) higher than 70° C. In another embodiment, said ankyrin repeat domain has an aggregation onset temperature (T_{agg}) higher than 72° C. In another embodiment, said ankyrin repeat domain has an aggregation onset temperature (T_{agg}) higher than 75° C. In another embodiment, said ankyrin repeat domain has an aggregation onset temperature (T_{agg}) higher than 78° C. In another embodiment, said ankyrin repeat domain has an aggregation onset temperature (T_{agg}) higher than 80° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein A at the second last position of SEQ ID NO: 1 is optionally substituted by L, and/or A at the last position of SEQ ID NO: 1 is optionally substituted by N; and wherein said ankyrin repeat domain has an aggregation onset temperature (T_{agg}) higher than 70° C. In one embodiment, said ankyrin repeat domain has an aggregation onset temperature (T_{agg}) higher than 75° C. In one embodiment, said ankyrin repeat domain has an aggregation onset temperature (T_{agg}) higher than 80° C. In one embodiment, said ankyrin repeat domain has an aggregation onset temperature (T_{agg}) higher than 85° C. In one embodiment, said ankyrin repeat domain has an aggregation onset temperature (T_{agg}) of between 75° C. and 100° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, wherein A at the second last position of SEQ ID NO: 2 is optionally substituted by L, and/or A at the last position of SEQ ID NO: 2 is optionally substituted by N; and wherein said ankyrin repeat domain has an aggregation onset temperature (T_{agg}) higher than 65° C. In one embodiment, said ankyrin repeat domain has an aggregation onset temperature (T_{agg}) higher than 70° C. In one embodiment, said ankyrin repeat domain has an aggregation onset temperature (T_{agg}) higher than 75° C. In one embodiment, said ankyrin repeat domain has an aggregation onset temperature (T_{agg}) higher than 80° C. In one embodiment, said ankyrin repeat domain has an aggregation onset temperature (T_{agg}) of between 70° C. and 95° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, wherein A at the second last position of SEQ ID NO: 3 is optionally substituted by L, and/or A at the last position of SEQ ID NO: 3 is optionally substituted by N; and wherein said ankyrin repeat domain has an aggregation onset temperature (T_{agg}) higher than 65° C. In one embodiment, said ankyrin repeat domain has an aggregation onset temperature (T_{agg}) higher than 70° C. In one embodiment, said ankyrin repeat domain has an aggregation onset temperature (T_{agg}) higher than 75° C. In one embodiment, said ankyrin repeat domain has an aggregation onset temperature (T_{agg}) higher than 80° C. In one embodiment, said ankyrin repeat domain has an aggregation onset temperature (T_{agg}) of between 70° C. and 95° C.

In one embodiment, said recombinant binding protein comprises an ankyrin repeat domain with binding specificity for CD3, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, wherein A at the second last position of SEQ ID NO: 4 is optionally substituted by L, and/or A at the last position of SEQ ID NO: 4 is optionally substituted by N; and wherein said ankyrin repeat domain has an aggregation onset temperature (T_{agg}) higher than 65° C. In one embodiment, said ankyrin repeat domain has an aggregation onset temperature (T_{agg}) higher than 70° C. In one embodiment, said ankyrin repeat domain has an aggregation onset temperature (T_{agg}) higher than 75° C. In one embodiment, said ankyrin repeat domain has an aggregation onset temperature (T_{agg}) higher than 80° C. In one embodiment, said ankyrin repeat domain has an aggregation onset temperature (T_{agg}) of between 70° C. and 95° C.

A typical and preferred determination of the aggregation onset temperature (T_{agg}) of the inventive recombinant binding proteins or ankyrin repeat domains with binding specificity for CD3 by Dynamic Light Scattering (DLS) analysis is described in Example 2. Thus, in one embodiment said aggregation onset temperature (T_{agg}) of the inventive recombinant binding proteins or ankyrin repeat domains is determined in TBS pH 8.0 (50 mM Tris, 500 mM NaCl) by Dynamic Light Scattering (DLS) analysis. In one embodiment said aggregation onset temperature (T_{agg}) of the inventive recombinant binding proteins or ankyrin repeat domains is determined in TBS pH 8.0 (50 mM Tris, 500 mM NaCl) by Dynamic Light Scattering (DLS) analysis as described in Example 2.

The repeat domains, preferably ankyrin repeat domains, of the recombinant binding protein disclosed herein preferably comprise a N-terminal and/or a C-terminal capping module (thereafter also referred to as capping repeats or capping units). Capping modules are located at the N-and/or C-terminal end of an ankyrin repeat domain, typically forming tight tertiary interactions (i.e. tertiary structure interactions) with the ankyrin repeat module(s) in between, thereby providing a cap that shields the hydrophobic core of the ankyrin repeat domain at the side from exposure to the solvent. The N-and/or C-terminal capping modules may be derived from a capping unit or other structural unit found in a naturally occurring repeat protein adjacent to a repeat unit. Examples of capping sequences are described in International Patent Publication Nos. WO 2002/020565 and WO 2012/069655, in U.S. Patent Publication No. US20130296221, and by Interlandi et al., J Mol Biol. 2008 Jan. 18; 375(3):837-54. Examples of N-terminal capping modules (i.e. N-terminal capping repeats) are SEQ ID NOs: 10-16 and examples of C-terminal capping modules (i.e. C-terminal capping repeats) are SEQ ID NOs: 17-26

In an exemplary embodiment, the N-terminal capping module comprises the amino acid sequence of any one of SEQ ID NOs: 10 to 15, wherein up to 9, up to 8, up to 7, up to 6, up to 5, up to 4, up to 3, up to 2 or up to 1 amino acid(s) of any one of SEQ ID NOs: 10 to 15 are optionally exchanged by any amino acids.

In an exemplary embodiment, the C-terminal capping module comprises the amino acid sequence of any one of SEQ ID NO: 17 to 25, wherein up to 9, up to 8, up to 7, up to 6, up to 5, up to 4, up to 3, up to 2 or up to 1 amino acid(s) of any one of SEQ ID NOs: 17 to 25 are optionally exchanged by any amino acids.

Advantageously, in some embodiments, certain amino acid residues in the N-terminal capping module and/or the C-terminal capping module of the designed ankyrin repeat domain herein provided are altered, resulting in improved pharmacokinetic properties, including a prolonged terminal half-life, of the designed ankyrin repeat domain and of the recombinant binding proteins comprising the designed ankyrin repeat domain. The altered amino acid residues are mostly surface exposed residues. Preferably, the altered amino acids residues are the amino acid residues at positions 8 and 15 of an N terminal capping module, wherein the amino acid at position 8 is Q and the amino acid at position 15 is L and wherein the position numbers correspond to the positions in SEQ ID NO: 10, and the amino acid residues at positions 14 and 18 of a C-terminal capping module, wherein the amino acid at position 14 is R and the amino acid at position 18 is Q and wherein the position numbers correspond to the positions in SEQ ID NO: 17.

For example, an N-terminal capping module with altered amino acid residues can comprise the following sequence: DLGxxLLQAAxxGQLDxVRxLxxxGADVNA (SEQ ID NO: 16), wherein “x” denotes any amino acid.

For example, a C-terminal capping module with altered amino acid residues can comprise the following sequence: xDxxGxTPADxAARxGHQxIAxVLQxAA (SEQ ID NO: 26), wherein “x” denotes any amino acid.

Accordingly, in one embodiment, the ankyrin repeat domain with binding specificity for CD3 of the invention comprises an N-terminal capping module having the amino acid sequence of SEQ ID NO: 16, wherein “x” denotes any amino acid. Alternatively or additionally, the ankyrin repeat domain with binding specificity for CD3 of the invention may comprise a C-terminal capping module having the amino acid sequence of SEQ ID NO: 26, wherein “x” denotes any amino acid.

In a particular embodiment, the ankyrin repeat domain with binding specificity for CD3 of the invention comprises the amino acid sequence of any one of SEQ ID NOs: 1 to 4, wherein the amino acid E at position 8 of any one of SEQ ID NOs: 1 to 4 is substituted with Q and the amino acid D at position 15 of any one of SEQ ID NOs: 1 to 3 is substituted with L. Alternatively or additionally, the ankyrin repeat domain with binding specificity for CD3 of the invention comprises the amino acid sequence of any one of SEQ ID NOs: 1 to 4, wherein the amino acid K at position 110 of any one of SEQ ID NOs: 1 to 4 is substituted with R and the amino acid E at position 114 of SEQ ID NOs: 1 and 2, or the amino acid R of SEQ ID NO: 4 is substituted with Q.

Furthermore, the CD3-binding domain of the invention may optionally further comprise a “G,” an “S,” or a “GS” sequence at its N-terminus. Accordingly, in some embodiments, the CD3-binding domain provided herein (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 to 4, and (ii) further comprises at its N-terminus, a G, an S, or a GS. In an exemplary embodiment, the CD3-binding domain comprises an amino acid sequence that is at least 90% identical to any one of SEQ ID NOs: 1 to 4, and further comprises at its N-terminus, a G, an S, or a GS. In an exemplary embodiment, the CD3-binding domain comprises an amino acid sequence that is at least 95% identical to any one of SEQ ID NOs: 1 to 4, and further comprises at its N-terminus, a G, an S, or a GS. In an exemplary embodiment, the CD3-binding domain comprises the amino acid sequence of any one of SEQ ID NOs: 1 to 4, and further comprises at its N-terminus, a G, an S, or a GS.

In one embodiment, said recombinant binding protein further comprises a binding agent with binding specificity for a disease-associated antigen.

Thus, in a particular embodiment, the recombinant binding protein of the invention comprises an ankyrin repeat domain with binding specificity for CD3 as described more specifically in any of the aspects or embodiments herein and further comprises a binding agent with binding specificity for a disease-associated antigen. In a preferred embodiment, said binding agent with binding specificity for a disease-associated antigen is located N-terminally of said ankyrin repeat domain with binding specificity for CD3 within said binding protein.

In one embodiment, said disease-associated antigen is a Tumor Associated Antigen (TAA). Illustrative examples of Tumor Associated Antigens include, but are not limited to, proteins expressed on the cell surface of tumor cells, such as, e.g., HER2 (also referred to as TAA1 herein), CD123 (also referred to as TAA2 herein), CD33 (also referred to as TAA3 herein), and CD70 (also referred to as TAA4 herein), or a peptide-MHC complex, wherein said peptide is derived from a protein expressed in a tumor cell (such as, e.g. NY-ESO-1 or MAGE-A3) and said MHC is MHC class I. In another embodiment, said disease-associated antigen is an Infection Associated Antigen, preferably a Virus Associated Antigen (VAA). Illustrative examples of Virus Associated Antigens include, but are not limited to, a peptide-MHC complex, wherein said peptide is derived from a viral infectious agent (such as, e.g. HBcAg or EBNA-1) and said MHC is MHC class I. In one embodiment, said binding agent with binding specificity for a disease-associated antigen is linked, conjugated, fused or otherwise physically attached to said ankyrin repeat domain with binding specificity for CD3. In one embodiment, said binding agent with binding specificity for a disease-associated antigen is covalently linked to said ankyrin repeat domain with binding specificity for CD3. In one embodiment, said binding agent with binding specificity for a disease-associated antigen is covalently linked to said ankyrin repeat domain with binding specificity for CD3 with a peptide linker. In one embodiment, the amino acid sequence of said peptide linker has a length from 1 to 50 amino acids, preferably from 6 to 38 amino acids. In one embodiment, said peptide linker is a proline-threonine rich peptide linker. In one embodiment, said peptide linker is the proline-threonine rich peptide linker of any one of SEQ ID NOs: 31 and 42 to 46. In one embodiment, said binding agent with binding specificity for a disease-associated antigen is covalently linked to said ankyrin repeat domain with binding specificity for CD3 with the proline-threonine rich peptide linker of any one of SEQ ID NOs: 31 and 42 to 46. In one preferred embodiment, said binding agent with binding specificity for a disease-associated antigen is a designed ankyrin repeat domain with binding specificity for a disease-associated antigen.

In one embodiment, the recombinant binding protein of the invention comprises an ankyrin repeat domain with binding specificity for CD3 as described more specifically in any of the aspects or embodiments herein and further comprises two ankyrin repeat domains with binding specificity for a disease-associated antigen as described more specifically in any of the aspects or embodiments herein. In a preferred embodiment, said two ankyrin repeat domains with binding specificity for a disease-associated antigen are located N-terminally of said ankyrin repeat domain with binding specificity for CD3 within said binding protein.

In one embodiment, said recombinant binding protein further comprises a half-life extending moiety.

Thus, in a particular embodiment, the recombinant binding protein of the invention comprises an ankyrin repeat domain with binding specificity for CD3 as described more specifically in any of the aspects or embodiments herein and further comprises a half-life extending moiety. In a preferred embodiment, said half-life extending moiety is located N-terminally of said ankyrin repeat domain with binding specificity for CD3 within said binding protein.

In one embodiment, said half-life extending moiety comprises a binding agent with binding specificity for human serum albumin.

In one embodiment, said half-life extending moiety is a designed ankyrin binding domain with binding specificity for human serum albumin. In one embodiment, said designed ankyrin binding domain with binding specificity for human serum albumin comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 28 to 30. Thus, in one embodiment, the recombinant binding protein of the invention comprises an ankyrin repeat domain with binding specificity for CD3 as described more specifically in any of the aspects or embodiments herein and further comprises an ankyrin binding domain with binding specificity for human serum albumin having an amino acid sequence with at least 80% amino acid sequence identity with any one of SEQ ID NOs: 28 to 30. In one embodiment, said ankyrin repeat domain with binding specificity for human serum albumin comprises an amino acid sequence with at least 90% amino acid sequence identity with any one of SEQ ID NOs: 28 to 30. In another embodiment, said ankyrin repeat domain with binding specificity for human serum albumin comprises an amino acid sequence with at least 93% amino acid sequence identity with any one of SEQ ID NOs: 28 to 30; and in a further embodiment, said ankyrin repeat domain with binding specificity for human serum albumin comprises an amino acid sequence with at least 95% amino acid sequence identity with any one of SEQ ID NOs: 28 to 30. In one embodiment, said ankyrin repeat domain with binding specificity for human serum albumin comprises an amino acid sequence with at least 98% amino acid sequence identity with any one of SEQ ID NOs: 28 to 30; and in a further embodiment, said ankyrin repeat domain with binding specificity for human serum albumin comprises the amino acid sequence of any one of SEQ ID NOs: 28 to 30. Thus, in one embodiment, the recombinant binding protein of the invention comprises an ankyrin repeat domain with binding specificity for CD3 and further comprises an ankyrin repeat domain with binding specificity for human serum albumin, and wherein ankyrin repeat domain with binding specificity for CD3 comprises the amino acid sequence of any one of SEQ ID NOs: 1 to 4, and wherein said ankyrin repeat domain with binding specificity for human serum albumin comprises the amino acid sequences of any one of SEQ ID NOs: 28 to 30. In a preferred embodiment, said ankyrin repeat domain with binding specificity for human serum albumin is located N-terminally of said ankyrin repeat domain with binding specificity for CD3 within said binding protein.

In a preferred embodiment, said designed ankyrin binding domain with binding specificity for human serum albumin comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 29. Thus, in one embodiment, the recombinant binding protein of the invention comprises an ankyrin repeat domain with binding specificity for CD3 as described more specifically in any of the aspects or embodiments herein and further comprises an ankyrin binding domain with binding specificity for human serum albumin having an amino acid sequence with at least 80% amino acid sequence identity with SEQ ID NO: 29. In one embodiment, said ankyrin repeat domain with binding specificity for human serum albumin comprises an amino acid sequence with at least 90% amino acid sequence identity with SEQ ID NO: 29. In another embodiment, said ankyrin repeat domain with binding specificity for human serum albumin comprises an amino acid sequence with at least 93% amino acid sequence identity with SEQ ID NO: 29; and in a further embodiment, said ankyrin repeat domain with binding specificity for human serum albumin comprises an amino acid sequence with at least 95% amino acid sequence identity with SEQ ID NO: 29. In one embodiment, said ankyrin repeat domain with binding specificity for human serum albumin comprises an amino acid sequence with at least 98% amino acid sequence identity with SEQ ID NO: 29; and in a further embodiment, said ankyrin repeat domain with binding specificity for human serum albumin comprises the amino acid sequence of SEQ ID NO: 29. Thus, in one embodiment, the recombinant binding protein of the invention comprises an ankyrin repeat domain with binding specificity for CD3 and further comprises an ankyrin repeat domain with binding specificity for human serum albumin, and wherein ankyrin repeat domain with binding specificity for CD3 comprises the amino acid sequence of any one of SEQ ID NOs: 1 to 4, and wherein said ankyrin repeat domain with binding specificity for human serum albumin comprises the amino acid sequences of SEQ ID NO: 29. In a preferred embodiment, said ankyrin repeat domain with binding specificity for human serum albumin is located N-terminally of said ankyrin repeat domain with binding specificity for CD3.

In one embodiment, a recombinant binding protein of the present invention comprising an ankyrin repeat domain with binding specificity for CD3 and further comprising an ankyrin repeat domain with binding specificity for human serum albumin exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for CD3 but not said ankyrin repeat domain with binding specificity for human serum albumin.

In one embodiment, the recombinant binding protein of the invention comprises an ankyrin repeat domain having binding specificity for CD3 as described more specifically in any of the aspects or embodiments herein and further comprises two ankyrin repeat domains with binding specificity for human serum albumin as described more specifically in any of the aspects or embodiments herein.

In one embodiment, the recombinant binding protein of the invention further comprises a polypeptide tag. A polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a binding protein may be connected to other parts of the binding protein directly or via a peptide linker. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one embodiment, the recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO:41, and examples of a proline-threonine-linker are the amino acid sequences of SEQ ID NOs: 31 and 42 to 46.

In another aspect, the invention relates to a nucleic acid encoding the amino acid sequence of an ankyrin repeat domain or a recombinant binding protein of the present invention. In one embodiment, the invention relates to a nucleic acid encoding the amino acid sequence of a recombinant binding protein of the present invention. In one embodiment, the invention relates to a nucleic acid encoding an amino acid sequence selected from the group consisting of SEQ ID NO: 1 to 4. In one embodiment, the invention relates to a nucleic acid encoding the amino acid sequence of SEQ ID NO: 1. In one embodiment, the invention relates to a nucleic acid encoding the amino acid sequence of SEQ ID NO: 2. In one embodiment, the invention relates to a nucleic acid encoding the amino acid sequence of SEQ ID NO: 3. In one embodiment, the invention relates to a nucleic acid encoding the amino acid sequence of SEQ ID NO: 4. Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli. Examples nucleic acids of the invention are provided by SEQ ID NOs: 37 to 40 which encode the amino acid sequences of SEQ ID NOs: 1 to 4, respectively.

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present invention, and optionally a pharmaceutically acceptable carrier and/or diluent.

In one embodiment, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient or stabilizer, for example as described in Remington's Pharmaceutical Sciences 16th edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

One embodiment of the present invention relates to the use of a recombinant binding protein of the present invention comprising an ankyrin repeat domain with binding specificity for CD3 and further comprising an ankyrin repeat domain with binding specificity for human serum albumin for manufacturing a pharmaceutical composition, wherein said recombinant binding protein exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for CD3 but not said ankyrin repeat domain with binding specificity for serum albumin. In one embodiment of the invention, a recombinant binding protein comprises an ankyrin repeat domain having binding specificity for CD3 and further comprises two ankyrin repeat domains with binding specificity for serum albumin.

In one embodiment, a pharmaceutical composition comprises at least one recombinant binding protein as described herein and a detergent such as nonionic detergent, a buffer such as phosphate buffer, and a sugar such as sucrose. In one embodiment, such a composition comprises recombinant binding proteins as described above and PBS.

In another aspect, the invention provides a method of CD3-mediated T cell activation in tumor cells or tissue in a mammal, including a human, the method comprising the step of administering to said mammal the inventive recombinant binding protein comprising an ankyrin repeat domain with binding specificity for CD3 and further comprising a localizer molecule. In one embodiment, said localizer molecule is a binding protein having binding specificity for a target different from CD3, preferably a TAA or a VAA. In one embodiment, said mammal is a human and said TAA expressing cells or tissue are located in a tumor, including in a primary tumor, metastasis and/or tumor stroma.

In another aspect, the invention provides a method of tumor-localized activation of T cells in a mammal, including a human, the method comprising the step of administering to said mammal the binding protein of the invention, the nucleic acid of the invention or the pharmaceutical composition of the invention.

In another aspect, the invention provides a method of infection-localized activation of T cells in a mammal, including a human, the method comprising the step of administering to said mammal the binding protein of the invention, the nucleic acid of the invention or the pharmaceutical composition of the invention.

In another aspect, the invention provides a method of treating a medical condition, the method comprising the step of administering to a patient in need thereof a therapeutically effective amount of the inventive recombinant binding protein further comprising a binding agent with binding specificity for a disease-associated antigen, a nucleic acid encoding said binding protein or a pharmaceutical composition comprising said binding protein. In another aspect, the invention provides a method of treating a medical condition, the method comprising the step of administering to a patient in need thereof a therapeutically effective amount of the inventive recombinant binding protein further comprising a binding agent with binding specificity for a disease-associated antigen, wherein said binding agent is effective in localizing said binding protein to target tumor tissue or infected tissue, and wherein said localization of said binding protein results in T cell activation in the target tumor or infected tissue. In one embodiment, said binding agent with binding specificity for a disease-associated antigen is a binding protein, preferably an ankyrin repeat binding protein, having binding specificity for a TAA or VAA.

In one embodiment, the invention relates to a pharmaceutical composition, a recombinant binding protein, or a nucleic acid according to the present invention for use in the treatment of a disease. For that purpose, the pharmaceutical composition, the nucleic acid or the recombinant binding protein according to the present invention is administered, to a patient in need thereof, in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above-mentioned pharmaceutical composition, nucleic acid or recombinant binding protein is considered for use in the treatment of a disorder.

In one embodiment, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one embodiment, the invention relates to a method of treatment of a medical condition, the method comprising the step of administering, to a patient in need of such a treatment, a therapeutically effective amount of the recombinant binding protein of the invention, the nucleic acid of the invention or the pharmaceutical composition of the invention. In one embodiment, the invention relates to the use of the recombinant binding protein of the invention, the nucleic acid of the invention or the pharmaceutical composition of the invention for the treatment of a disease. In one embodiment, the invention relates to the recombinant binding protein of the invention, the nucleic acid of the invention or the pharmaceutical composition of the invention for use in the treatment of a disease. In one embodiment, the invention relates to the recombinant binding protein of the invention, the nucleic acid of the invention or the pharmaceutical composition of the invention for use in the treatment of a medical condition. In one embodiment, the invention relates to the use of the recombinant binding protein of the invention, the nucleic acid of the invention or the pharmaceutical composition of the invention, as medicament for the treatment of a disease. In one embodiment, the invention relates to the use of the recombinant binding protein of the invention, the nucleic acid of the invention or the pharmaceutical composition of the invention for manufacturing of a medicament. In one embodiment, the invention relates to the use of the recombinant binding protein of the invention, the nucleic acid of the invention or the pharmaceutical composition of the invention, for manufacturing of a medicament for the treatment of a disease. In one embodiment, the invention relates to a process for the manufacturing of a medicament for the treatment of a disease, wherein the recombinant binding protein of the invention, the nucleic acid of the invention or the pharmaceutical composition of the invention is an active ingredient of the medicament. In one embodiment, the invention relates to a method of treatment of a disease using the recombinant binding protein of the invention, the nucleic acid of the invention or the pharmaceutical composition of the invention.

The use of a recombinant binding protein of the present invention, a nucleic acid of the invention or a pharmaceutical composition of the invention for the treatment of cancer or infectious diseases can also be in combination with one or more other therapies known in the art. The term “use in combination with”, as used herein, shall refer to a co-administration, which is carried out under a given regimen. This includes synchronous administration of the different compounds as well as time-shifted administration of the different compounds (e.g. compound A is given once and compound B is given several times thereafter, or vice versa, or both compounds are given synchronously and one of the two is also given at later stages).

In one embodiment said medical condition is cancer. In another embodiment, said medical condition is an infectious disease, preferably a viral infectious disease.

In one particular embodiment, the medical condition is cancer, wherein the cancer or tumor tissue comprises cells that express or display a tumor-associated antigen, and said binding agent with binding specificity for a disease-associated antigen binds said tumor-associated antigen expressed or displayed in said cells. In one particular embodiment, said disease-associated antigen is the extracellular domain of a cell surface protein expressed or overexpressed in said cancer or tumor tissue. In another particular embodiment, said binding agent with binding specificity for a tumor-associated antigen binds to a peptide-MHC complex, wherein said peptide is derived from a protein expressed in a tumor cell (such as, e.g. NY-ESO-1 or MAGE-A3). In one particular embodiment, said MHC is MHC class I. In one embodiment, said cancer is selected from adenocarcinoma and squamous cell carcinoma. In one embodiment, said cancer is selected from osteosarcoma or osteogenic sarcoma (bone), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), and mesenchymous or mixed mesodermal tumor (mixed connective tissue types). In one embodiment, said cancer is selected from myelogenous or granulocytic leukemia (malignancy of the myeloid and granulocytic white blood cell series), lymphatic, lymphocytic, or lymphoblastic leukemia (malignancy of the lymphoid and lymphocytic blood cell series), and polycythemia vera or erythremia (malignancy of various blood cell products, but with red cells predominating). In one embodiment, said cancer is selected from Hodgkin lymphoma and non-Hodgkin lymphoma. In one embodiment, said cancer is selected from adenosquamous carcinoma, mixed mesodermal tumor, carcinosarcoma and teratocarcinoma. In one embodiment, said cancer is selected from colorectal cancers, gastric cancers, non-small cell lung cancers, breast cancers, head and neck cancer, ovarian cancers, lung cancers, invasive bladder cancers, pancreatic cancers, metastatic cancers of the brain, head and neck squamous cell carcinoma, esophagus squamous cell carcinoma, lung squamous cell carcinoma, skin squamous cell carcinoma, melanoma, breast adenocarcinoma, lung adenocarcinoma, cervix squamous cell carcinoma, pancreas squamous cell carcinoma, colon squamous cell carcinoma, or stomach squamous cell carcinoma, prostate cancer, osteosarcoma or soft tissue sarcoma and benign tumors. In one embodiment, such cancer is selected from epithelial malignancies (primary and metastatic), including lung, colorectal, gastric, bladder, ovarian and breast carcinomas, and bone and soft tissue sarcomas.

In another particular embodiment, the medical condition is an infectious disease, preferably a viral infectious disease, wherein the infected tissue comprises cells that express or display an infection-associated antigen, preferably a virus-associated antigen, and said binding agent with binding specificity for a disease-associated antigen binds said infection-associated antigen expressed or displayed in said cells. In one particular embodiment, said disease-associated antigen is the extracellular domain of a virus protein expressed or overexpressed in said infected tissue. In another particular embodiment, said binding agent with binding specificity for a virus-associated antigen binds to a peptide-MHC complex, wherein said peptide is derived from a protein of an infectious agent, such as a bacterial infectious agent or a viral infectious agent, preferably a viral infectious agent (such as, e.g. HBcAg or EBNA-1). In one particular, embodiment, said MHC is MHC class I. In one embodiment, said infectious disease is selected from a viral infection caused by hepatitis B virus (HBV), a viral infection caused by Epstein-Barr virus (EBV), HIV infection, West Nile virus infection, hepatitis A, B, and C, small pox, tuberculosis, Vesicular Stomatitis Virus (VSV) infection, Respiratory Syncytial Virus (RSV) infection, human papilloma virus (HPV) infection, SARS, influenza, Ebola, viral meningitis, herpes, anthrax, lyme disease, and E. coli infections. In one embodiment the invention relates to a recombinant binding protein comprising any of the above mentioned ankyrin repeat domains.

In one embodiment, the invention relates to a kit comprising the recombinant binding protein of the invention. In one embodiment, the invention relates to a kit comprising a nucleic acid encoding the recombinant binding protein of the invention. In one embodiment, the invention relates to a kit comprising the pharmaceutical composition of the invention. In one embodiment, the invention relates to a kit comprising the recombinant binding protein of the invention, and/or the nucleic acid of the invention, and/or the pharmaceutical composition of the invention. In one embodiment, the invention relates to a kit comprising the recombinant binding protein comprising an ankyrin repeat domain with binding specificity for CD3 of the invention, for example SEQ ID NOs: 1 to 4 and/or a nucleic acid encoding the recombinant binding protein comprising an ankyrin repeat domain with binding specificity for CD3, for example SEQ ID NOs: 1 to 4, and/or a pharmaceutical composition comprising the recombinant binding protein comprising an ankyrin repeat domain with binding specificity for CD3, for example SEQ ID NOs: 1 to 4.

In one embodiment, the invention relates to a method for producing a recombinant binding protein of the present invention. In one embodiment, the invention relates to a method for producing a recombinant binding protein, for example a recombinant binding protein comprising the amino acid sequence of SEQ ID NOs: 1 to 4, the method comprising the steps of (i) expressing said recombinant binding protein in a suitable host cell (e.g., bacteria), and (ii) purifying said recombinant binding protein (e.g., using chromatography). Said method may comprise additional steps. Such a method of producing a recombinant binding protein of the present invention is described in Example 1.

The invention is not restricted to the particular embodiments described in the Examples.

This specification refers to a number of amino acid sequences, nucleic acid sequences and SEQ ID NOs that are disclosed in the appended Sequence Listing, which is herewith incorporated by reference in its entirety.

### Definitions

Unless defined otherwise herein, all technical and scientific terms used herein shall have the meanings that are commonly understood by those of ordinary skill in the art to which the present invention belongs.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QlAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for CD3.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins (e.g. DM1 from Immunogen), small molecules, antibiotics and alike) well known to the person skilled in the art. A binding protein of the instant invention may comprise a localizer molecule.

The term “binding domain” means a protein domain exhibiting binding specificity for a target. Preferably, said binding domain is a recombinant binding domain.

The term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, T cells are targets of CD3-specific binding proteins and localizer target proteins and cells and tissues are targets of localizers.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

Patent application WO2002/020565 and Forrer et al., 2003 (Forrer, P., Stumpp, M. T., Binz, H. K., Pluckthun, A., 2003. FESS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain.

The term “target interaction residues” refers to amino acid residues of a repeat module, which contribute to the direct interaction with a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or 6-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target, e.g. as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. These positions are generally not substituted in the variants of the module sequences of SEQ ID Nos: 5 to 9 disclosed herein.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “has binding specificity for a target”, “specifically binding to a target”, “binding to a target with high specificity”, “specific for a target” or “target specificity” and the like means that a binding protein or binding domain binds in PBS to a target with a lower dissociation constant (i.e. it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10³ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the inventive recombinant binding proteins with binding specificity for CD3 by Surface Plasmon Resonance (SPR) analysis is described in Example 4.

The term “binding agent” refers to any molecule capable of specifically binding a target molecule. Binding agents include, for example, antibodies, antibody fragments, aptamers, peptides (e.g., Williams et al., J Biol Chem 266:5182-5190 (1991)), antibody mimics, repeat proteins, e.g. designed ankyrin repeat proteins, receptor proteins and any other naturally occurring interaction partners of the target molecule, and can comprise natural proteins and proteins modified or genetically engineered, e.g., to include non-natural residues and/or to lack natural residues.

The term “about” means the mentioned value+/−20%; for example “about 50” shall mean 40 to 60.

The term “PBS” means a phosphate buffered water solution containing 137 mM NaCl, 10 mM phosphate and 2.7 mM KCl and having a pH of 7.4.

The term “mouse serum albumin” refers to UniProt accession number P07724, the term “cynomolgus monkey serum albumin” (i.e. Macaca fascicularis) refers to UniProt accession number A2V9Z4, and the term “human serum albumin” refers to UniProt accession number P02768.

Preferably, clearance, and/or exposure, and/or terminal half-life are assessed in a mammal, more preferably mouse and/or cynomolgus monkey, more preferably cynomolgus monkey. Clearance, and/or exposure, and/or terminal half-life may be assessed as described in Example 3. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art. Preferably, pharmacokinetic comparison is done at any dose, more preferably at equivalent dose (i.e. same mg/kg dose) or equimolar dose (i.e. same mol/kg dose), more preferably at equimolar dose (i.e. same mol/kg dose). It is understood by the person skilled in the art that equivalent and/or equimolar dosing in animals is subject to experimental dose variations of at least 20%, more preferably 30%, 40%, 50%, 60%, 70%, 80%, 90%, or 100%. Preferably, a dose used for pharmacokinetic measurement is selected from 0.001 to 1000 mg/kg, more preferably 0.01 to 100 mg/kg, more preferably 0.1 to 50 mg/kg, more preferably 0.5 to 10 mg/kg.

The term “CD3” or “Cluster of Differentiation 3” refers to a multimeric protein complex composed of four distinct polypeptide chains, epsilon (ε), gamma (γ) and zeta (ζ) that assemble as three pairs (εγ, εδ, ζζ). The CD3 complex serves as a T cell co-receptor that associates non-covalently with the T cell receptor. It may refer to any form of CD3, as well as to variants, isoforms, and species homologs thereof that retain at least a part of the activity of CD3. Accordingly, a binding protein, as defined and disclosed herein, may also bind CD3 from species other than human. In other cases, a binding protein may be completely specific for the human CD3 and may not exhibit species or other types of cross-reactivity. Unless indicated differently, such as by specific reference to human CD3, CD3 includes all mammalian species of native sequence CD3, e.g., human, canine, feline, equine and bovine. The amino acid sequences of human CD3 gamma, delta and zeta chains are shown in NCBI (www.ncbi.nlm.nih.gov/) Ref. Seq. NP_000064.1, NP_000723.1 and NP_932170.1, respectively.

The term “CD3-expressing cells” as used herein refers to any cells expressing CD3 (cluster of differentiation 3) on the cell surface, including, but not limited, to T cells such as cytotoxic T cells (CD8+ T cells) and T helper cells (CD4+ T cells).

The term “tumor-localized activation of T cells” means that T cells are activated preferentially in tumor tissue as compared to a non-tumor tissue.

The term “infection-localized activation of T cells” means that T cells are activated preferentially in an infected tissue as compared to a non-infected tissue.

Furthermore, the term “peptide” also encompasses peptides modified by, e.g, glycosylation, and proteins comprising two or more polypeptide chains, each of length of 4 to 600 amino acids long, cross-linked by, e.g., disulphide bonds, such as, e.g., insulin and immunoglobulins. The term “chemical or biochemical agent” is intended to include any naturally occurring or synthetic compound that may be administered to a recipient.

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, and a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is a cancer. The term “therapeutically effective amount” means an amount that is sufficient to produce a desired effect on a patient.

The term “antibody” means not only intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)2, and single chain V region fragments (scFv), bispecific antibodies, chimeric antibodies, antibody fusion polypeptides, and unconventional antibodies.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

The terms “infectious disease” and “infection” are used herein to refer to or describe the invasion and multiplication of microorganisms in body tissues, especially causing pathological symptoms. Examples of infectious diseases include without limitation, viral diseases and bacterial diseases, such as, e.g., HIV infection, West Nile virus infection, hepatitis A, B, and C, small pox, tuberculosis, Vesicular Stomatitis Virus (VSV) infection, Respiratory Syncytial Virus (RSV) infection, human papilloma virus (HPV) infection, SARS, influenza, Ebola, viral meningitis, herpes, anthrax, lyme disease, and E. coli infections, among others.

### EXAMPLES

Starting materials and reagents disclosed below are known to those skilled in the art, are commercially available and/or can be prepared using well-known techniques.

Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA).

Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989,

New York).

Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 10, 11, 12, 13, or 15) or a randomized N-terminal capping module according to SEQ ID NO: 16, and a fixed C-terminal capping module (e.g. the C-terminal capping module of SEQ ID NO: 17, 18, 19, 20, 21, 22, 23, 24 or 25) or a randomized C-terminal capping module according to SEQ ID NO: 26. Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RILLAA, RILLKA or RELLKA motif (e.g. present from position 21 to 26 in SEQ ID NO: 1) and any such C-terminal capping module of an ankyrin repeat protein library preferably possesses the KLN, KLA or KAA motif (e.g. present at the last three amino acids in SEQ ID NO: 1). SEQ ID NOs: 10, 11, 12, 13, 14, or 15 provide examples of N-terminal capping modules comprising the RILLAA, RILLKA or RELLKA motif, and SEQ ID NOs: 17, 18, 19, 20, 21, 22, 23, 24 or 25 provide examples of C-terminal capping modules comprising the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V20, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

All the DNA sequences were confirmed by sequencing, and the calculated molecular weight of selected proteins was confirmed by mass spectrometry.

### Example 1: Selection of Binding Proteins Comprising an Ankyrin Repeat Domain with Binding Specificity for CD3

Using ribosome display (Hanes, J. and Plückthun, A., PNAS 94, 4937-42, 1997), many ankyrin repeat proteins with binding specificity for human scCD3 were selected from DARPin® libraries similar as described by Binz et al. 2004 (loc. cit.). The binding of the selected clones towards recombinant human CD3 target was assessed by crude extract Homogeneous Time Resolved Fluorescence (HTRF), indicating that hundreds of human scCD3-specific binding proteins were successfully selected. For example, the ankyrin repeat domain of SEQ ID NO: 1 constitutes an amino acid sequence of a selected binding protein comprising an ankyrin repeat domain with binding specificity for scCD3. Individual ankyrin repeat modules from such ankyrin repeat domains with binding specificity to scCD3 are provided, e.g., in SEQ ID NO: 5 to 9.

Human Recombinant CD3 Target Preparation

The target format chosen is based on single chain format, consisting of the human CD3E and CD3γ heterodimer linked by a 26 amino acid linker (scCD3εγ) and a C-terminal Avi-tag for site-directed biotinylation. The target protein contains only the CD3 extracellular domain, lacking the C-terminal cysteine “knobs” and the entire transmembrane and cytoplasmic regions.

The extracellular domain of human scCD3εγ (SEQ ID NO: 32_scCD3εγ _Avi-Bio) was expressed in a single-chain format similar as described previously (Kjer-Nielsen et al., PNAS, 2004, 101 (20):7675-7680) in Escherichia coli, followed by refolding from inclusion bodies and purified by preparative size exclusion chromatography (SEC). The material was up-concentrated in 10 mM Tris-HCl, 50 mM NaCl, pH 8.0 to 3.4 mg/ml and in vitro biotinylated using recombinant BirA. To isolate functional target material, the material was re-purified using an OKT3-loaded column (GE HiTrap NHS-activated HP column). The final material was monomeric on size exclusion and stored at the final concentration of 0.39 mg/ml in 10 mM Tris, 100 mM NaCl, pH 8.0, 10% glycerol.

An overview of the recombinant human scCD3 target preparation process is described in Dunstone et al., Acta Crystallographica 2004.

Selection of CD3-Specific Ankyrin Repeat Proteins by Ribosome Display

The selection of CD3-specific ankyrin repeat proteins was performed by ribosome display (Hanes and Plückthun, loc. cit.) using part of the extracellular domain of CD3 (SEQ ID NO: 32) as target protein, libraries of ankyrin repeat proteins as described above, and established protocols (see, e.g., Zahnd, C., Amstutz, P. and Pluckthun, A., Nat. Methods 4, 69-79, 2007). The number of reverse transcription (RT)-PCR cycles after each selection round was constantly reduced from 45 to 28, adjusting to the yield due to enrichment of binders. The first four rounds of selection employed standard ribosome display selection, using decreasing target concentration (400 nM, 133 nM, 45 nM and 15 nM, respectively) and increasing washing stringency to increase selection pressure from round 1 to round 4 (Binz et al. 2004, loc. cit.). In rounds 2-4, mRNA was recovered by competitive elution using excess of CD3 binding antibody OKT3 (in each round, competitor excess was constantly increased from 35-fold to 300-fold).

Selected Clones Activate T-Cells in a Bivalent Format

Individual ankyrin repeat protein clones binding to CD3 target were selected by ribosome display and were cloned into derivatives of the pQE30 (Qiagen) expression vector, transformed into E. coli XL1-Blue (Stratagene), plated on LB-agar (containing 1% glucose and 50 μg/ml ampicillin) and then incubated overnight at 37° C. The expression vector, a Jun leucine-zipper construct with both His- and Myc-tag and a CD3-specific ankyrin repeat domain, was used for screening in a bivalent format (with regard to the CD3-specific binding domain), which allowed testing for functionality by cross-linking of T-cells. Single colonies were picked into a 96 well plate (each clone in a single well) containing 160 μl growth medium (TB containing 1% glucose and 50 μg/ml ampicillin) and incubated overnight at 37° C., shaking at 800 rpm. 150 μl of fresh TB medium containing 50 μg/ml ampicillin was inoculated with 8.5 μl of the overnight culture in a fresh 96 well plate. After incubation for 120 minutes at 37° C. and 850 rpm, expression was induced with IPTG (0.5 mM final concentration) and continued for 4 hours. Cells were harvested and the pellets were frozen at −20° C. overnight before resuspension in 8.5 μl μl B-PERII (Thermo Scientific) and incubation for one hour at room temperature with shaking (600 rpm). Then, 160 μl PBS was added and cell debris was removed by centrifugation (3220 g for 15 min), and stored at −20° C. for further usage.

In a first step, a T-cell activation screen was performed using BK112 CD8+ monoclonal T-cells. The extract of each lysed clone was applied as a 1:20 dilution (final concentration) in PBSB (PBS pH 7.4 supplemented with 12% (w/v) FBS) to an anti-penta-His-antibody (Qiagen) coated 96 well plate, and incubated at 4° C. overnight. Plates were washed five times wish PBS before 100 μl of 100′000 BK112 T cells were added per well, cultured in T-cell assay medium RPMI-1640+10% FBS+1% L-glutamine+1% Pen Strep+200IUIL2. 0.1 μg/100 μL of Golgi Stop were added and plates were centrifuged at 20 g for 3 minutes at RT before incubation of 4-5 hours at 37° C. in the CO₂-incubator. Cells were centrifuged at 350 g for 5 minutes at 4° C. and decanted. Cells were stained for surface CD8 expression before preserving the cells using BD Cytofix, incubated overnight at 4° C. Cells were washed with 1×PBS+2% FBS and stained for intracellular IFNγ by adding 50 μl of IFNγ-APC antibody in Cell perm (BD) and incubation for 30 min at 4° C. Cells were washed again in PBS and analyzed using a Cytometer FACS Canto II from BD.

Selected Clones Show Binding to CD3 (Shown by HTRF and OKT3 Competition) and Functionality in the Bispecific Format

Identified functional designed ankyrin repeat domains hits were subcloned into derivatives of the pQE30 (Qiagen) expression vector containing an N-terminal His-tag, a Tumor Associated Antigen 1 (TAA1)-specific ankyrin repeat domain and a CD3-specific ankyrin repeat domain, in order to create a T cell engager (TCE) construct. Constructs were expressed in E. coli cells and purified using their His-tag according to standard protocols. 25 ml of stationary overnight cultures (TB, 1% glucose, 50 μg/ml of ampicillin; 37° C.) were used to inoculate 500 ml cultures (TB, 50 μg/ml ampicillin, 37° C.). At an absorbance of 1.0 to 1.5 at 600 nm, the cultures were induced with 0.5 mM IPTG and incubated at 37° C. for 4-5 h while shaking. The cultures were centrifuged and the resulting pellets were re-suspended in 25 ml of TBS₅₀₀ (50 mM Tris-HCl, 500 mM NaCl, pH 8) and lysed (sonication). Following the lysis, the samples were mixed with 50 KU DNase/ml and incubated for 15 minutes prior to a heat-treatment step for 30 minutes at 62.5° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art.

In a first step, binding to recombinant protein was tested using an HTRF assay. Titration of the ankyrin repeat protein (5−640 nM) in PBS-TC (PBS supplemented with 0.1% (w/v) Casein and 0.1% Tween20, pH 7.4) was performed against 48 nM (final concentration) of human biotinlyated scCD3εγ, 1:100 (final concentration) of anti-6His-D2 HTRF antibody—FRET acceptor conjugate (Cisbio) and 1:100 (final concentration) of anti-strep-Tb antibody FRET donor conjugate (Cisbio) in a well of a 384-well plate and incubated for 120 minutes at RT. The HTRF was read-out on a Tecan M1000 using a 340 nm excitation wavelength and a 665±10 nm emission filter. Several candidates showed dose dependent binding and were used for further evaluation. For all these constructs, binding signals were at background level when competed with 20-fold excess of CD3 binding antibody (OKT3 variant containing a human Fc region—final concentration 2.4 mM), which binds to a conformational epitope of CD3E (Kjer-Nielsen et al., PNAS, 2004, 101 (20):7675-7680). Dose-dependent in vitro T-cell activation was confirmed using a BK112 T-cell activation assay (BK112 CD8 monoclonal T-cells which were pre-activated with CD3/CD28 Dynabeads), in presence of TAA1 expressing tumor cells. Intracellular IFNγ levels were measured on CD8+ or CD4+ T-cells after 5 hours of incubation of BK112 and SKOV3 cells (E:T=1:10) in presence of 1-100′000 pM of bispecific TCE constructs. The most potent construct showed EC₅₀ values of 0.5 nM and 0.4 nM for CD4+ and CD8+ cells, respectively.

Affinity Maturation and Rational Design of Selected CD3-Specific Ankyrin Repeat Proteins

Several rounds of affinity maturation combined with rational design were applied on the parental low affinity binding CD3-specific ankyrin repeat protein (named precursor A), and resulted in four higher affinity CD3-specific ankyrin repeat proteins (named precursor B, C and D). These precursor molecules were then finally engineered into CD3-specific ankyrin repeat proteins DARPin® protein #1, DARPin® protein #2, DARPin® protein #3 and DARPin® protein #4.

Affinity maturation was performed on one of the parental CD3-specific ankyrin repeat proteins (precursor A), which was chosen taking into consideration, both its sufficient binding ability to the CD3 target and its ability to efficiently activate T-cells in vitro), by introducing diversity using error-prone PCR and DNA I shuffling as described by Zahnd et al., Nat Methods, 2007, 4: 269-279. In short: Three rounds of ribosome display were conducted using different concentrations of dNTP-analogues (mutagenesis kit from Jena Biosciences, using 5-10 pM 8-oxo-dGTP and dPTP) to introduce approximately 1-2 mutations per CD3-specific ankyrin repeat protein/round with increasing selection pressures (washing steps were increased from round 1 (3×15 min), to 3×30 min (round 2), to 3×45 min (round3)), while the target concentration was kept constant at 5 nM. In a second step, DNA pools were DNA-shuffled and back-crossed using parental clones in one or two rounds of ribosome display using as described previously (Cadwell & Joyce, PCR Methods Appl, 1992, 2:28-33; Stemmer, Nature, 1994 370:389-391; Zaccolo et al, J Mol Biol 1996, 255: 589-603) using a DNAse I incubation time of 90 seconds and DNA polymerase HotStarTaq DNA Polymerase (Qiagen). Affinity matured CD3-specific ankyrin repeat protein pools were subcloned into derivatives of the pQE30 (Qiagen) expression vector, finally containing an N-terminal His-Flag-tag, an HSA binding ankyrin repeat domain for half-life extension, a TAA1-binding ankyrin repeat domain and a CD3-specific ankyrin repeat domain, and expressed and screened for binding to recombinant scCD3εγ by HTRF as described before. A lead clone was selected (precursor B, generated after 5 rounds of affinity maturation including 3 rounds of error-prone PCR using in all steps 10 μM dNTP analogues and two rounds of DNA-shuffling and back-crossing).

In the same process, several potential beneficial mutations for increased T-cell activation were identified by % of IFNγ+ T-cell in a BK112 assay, including N-cap mutations in positions 5 and 20, 1st internal repeat mutations in positions 2 and 4, 2^{nd} internal repeat mutations in position 2, 5 and 20, and C-cap mutations in positions 1 and 18. A combination of these mutations—while keeping N- and the C-cap framework mutations to a minimum in order to maintain thermal stability—resulted in a set of designed variants, which were tested again in the same format for improved T-cell activation. Thereby, a new further matured variant (precursor C) was generated with higher T-cell activation potency compared to parental and initially matured ones.

In order to increase CD3 affinity and T-cell activation potency even further, a second affinity maturation was performed on the precursor C clone, similar to what has been described above. In short: Four rounds of ribosome display were performed. In the first three rounds, mutations were introduced by error-prone PCR with 7.5 μM of dNTP analogues. In round one and three, an off-rate selection was applied using either the further matured clone itself or a non-biotinylated CD3 target protein for competition. 1 nM target was used in round 1 and 2, whereas 5 nM was applied in less stringent rounds 3 and 4. CD3 variants were screened in a trispecific format, including an HSA-binding domain and a TAA1 binding ankyrin repeat domain, using an off-rate HTRF assay with 250-fold access of the further matured clone as competitor. A total of 3×96 clones with highest remaining HTRF signal after competition were sequenced. Identified beneficial mutations for improved binding (including N-cap position 16, first internal repeat positions 1, 12, 18, 19, 26, 30 and 33, second internal repeat positions 2, 3, 7, 20, 21, 26 and 32, and C-cap positions 9, 11 and 18) or for reduced domain interactions (including N-cap positions 11, 18, 19 26, and C-cap positions 3, 19 and 22) were tested first as individual or paired mutations on purified proteins variants for BK112 T-cell activation. Most beneficial variants were then recombined in a second step and variants were screened for highest T-cell activation, which resulted in the identification of precursor D.

In a last step, the variants DARPin® protein #1, DARPin® protein #2, DARPin® protein #3 and DARPin® protein #4 were generated based on CD3-specific ankyrin repeat protein precursor A, B, C and D, respectively, in order to improve serum half-life and biophysical properties. Thereby, N-cap mutations in positions 23 and/or 26 were introduced while some of the framework mutations were removed (in positions 19, 18).

Affinity matured ankyrin repeat domains with binding specificity for human CD3 were cloned into a pQE (QlAgen, Germany) based expression vector providing an N-terminal His-tag (SEQ ID NO:27) to facilitate simple protein purification as described below. For example, expression vectors encoding the following ankyrin repeat proteins were constructed:
DARPin® protein #1 (SEQ ID NO:1 with a His-tag (SEQ ID NO: 27) fused to its N terminus);
DARPin® protein #2 (SEQ ID NO:2 with a His-tag (SEQ ID NO: 27) fused to its N terminus);
DARPin® protein #3 (SEQ ID NO:3 with a His-tag (SEQ ID NO: 27) fused to its N terminus);
DARPin® protein #4 (SEQ ID NO:4 with a His-tag (SEQ ID NO: 27) fused to its N terminus);

High Level and Soluble Expression of CD3-Specific Ankyrin Repeat Proteins

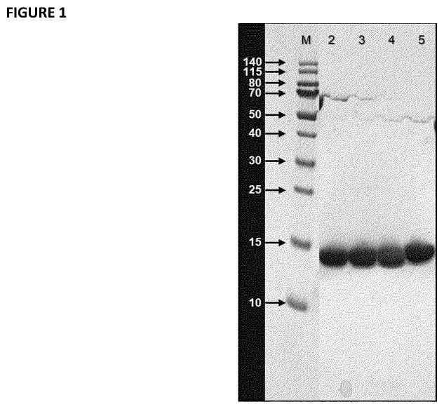

For in-depth analyses, the selected clones showing specific CD3 binding, either as monovalent or in combination with TAAs and/or HSA binding ankyrin repeat domains were expressed in E. coli cells and purified using their His-tag followed by a size exclusion chromatography on an ÄKTAxpress™ system according to standard protocols and resins known to the person skilled in the art. The proteins were monomeric and soluble when concentrated to 10 mg/ml in TBS pH 8.0 (50 mM Tris, 500 mM NaCl) or PBS pH 7.4 for monovalent and multivalent constructs, respectively. A representative example of such SDS-PAGE analysis is shown in FIG. 1.

### Example 2: Stability Assessment of Exemplary CD3-Specific Ankyrin Repeat Proteins

Assessment of Exemplary CD3-Specific Ankyrin Repeat Proteins Using UV Spectroscopy, SDS-PAGE and Size-Exclusion Chromatography (SEC)

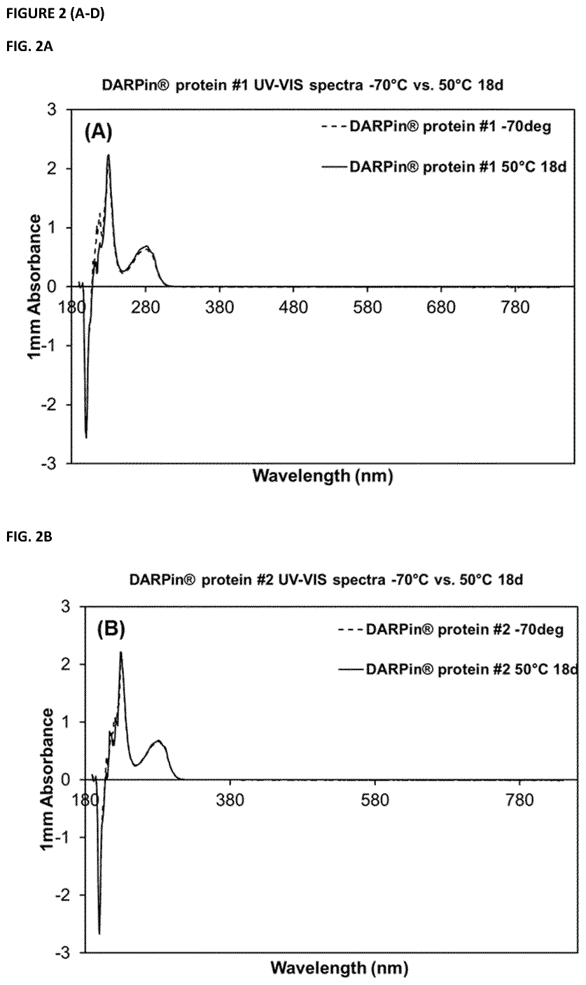

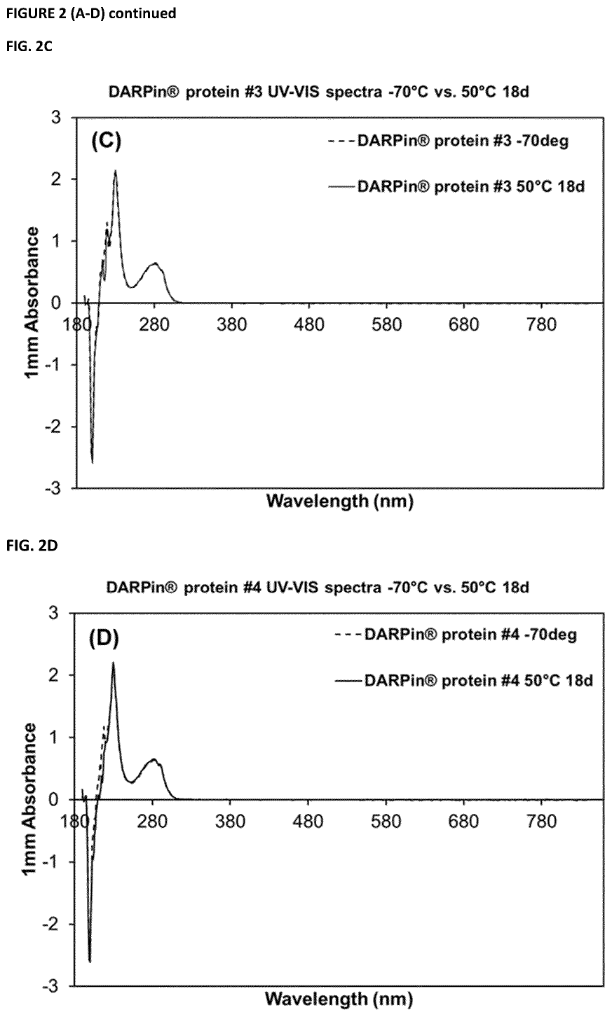

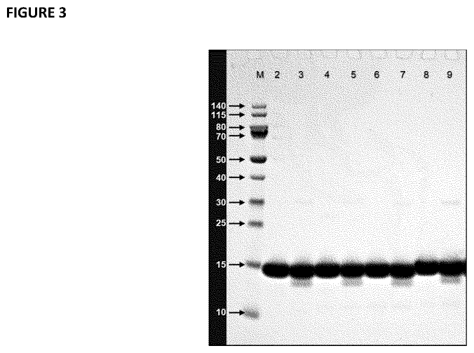

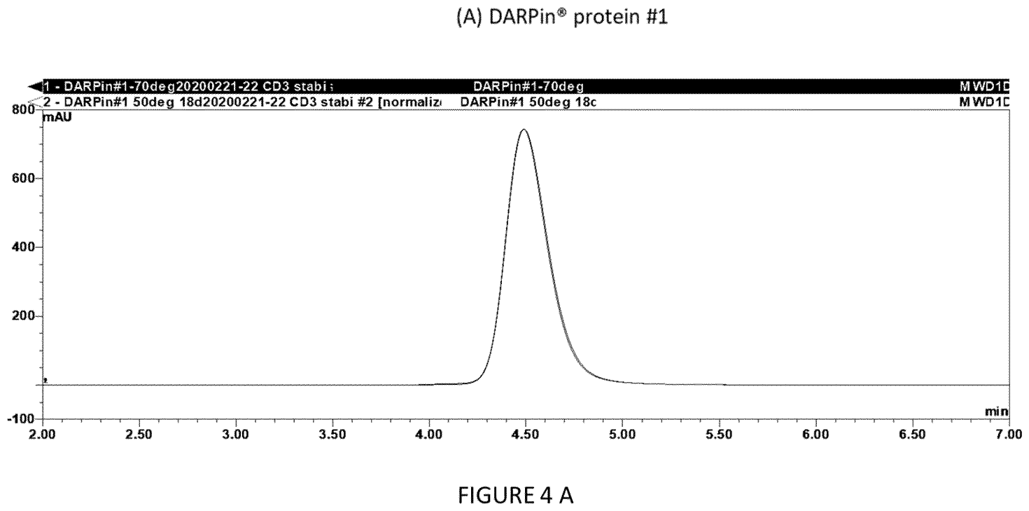

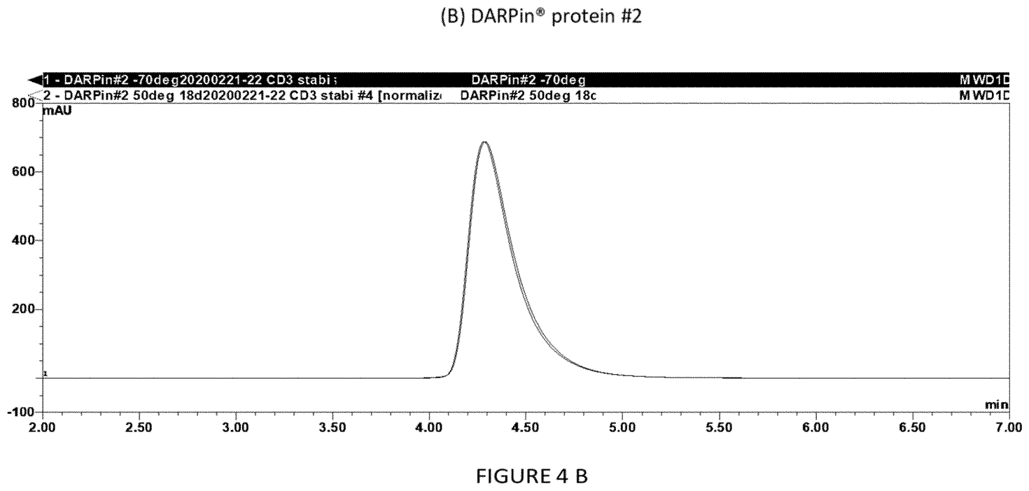

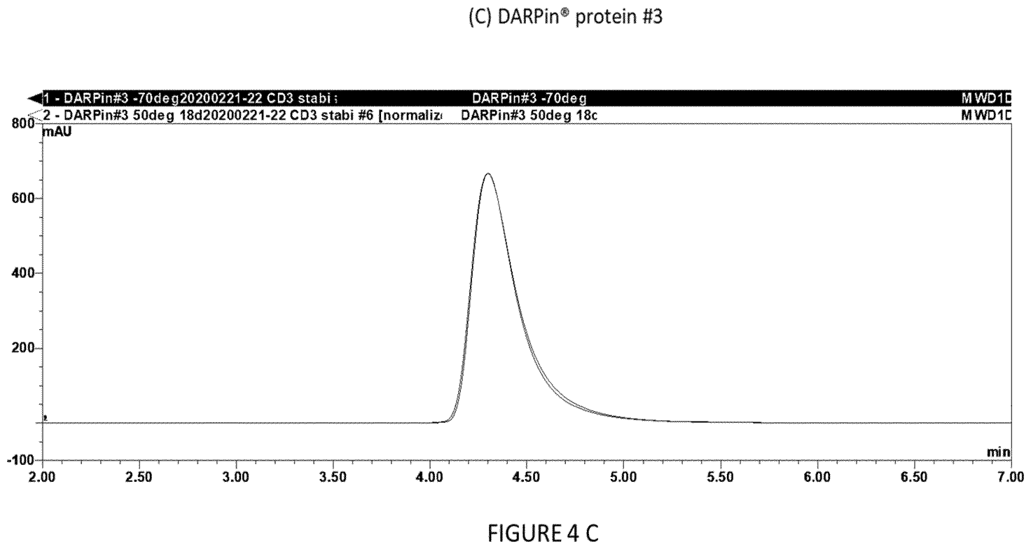

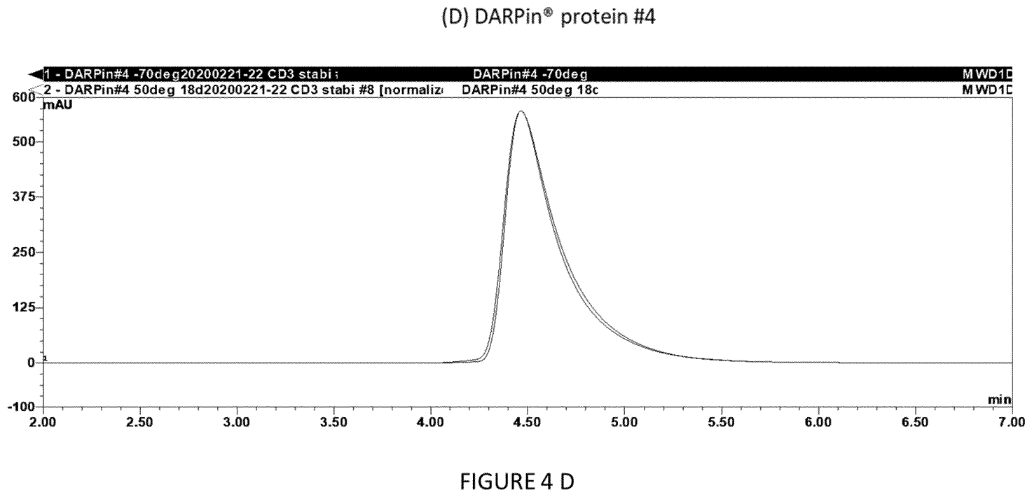

Four selected CD3-specific ankyrin repeat proteins were analysed in-depth to assess their biophysical properties. In brief, purified CD3-specific ankyrin repeat proteins DARPin® #1 (SEQ ID NO: 1), DARPin® #2 (SEQ ID NO: 2), DARPin® #3 (SEQ ID NO: 3) and DARPin® #4 (SEQ ID NO: 4) in TBS pH 8.0 (50 mM Tris, 500 mM NaCl) were aliquoted into sterile glass vials (Schmidlin: LPP 1109 0620) and stressed by incubation at 50° C. for 18 days. The applied stress conditions allow a prediction of the biophysical properties after 2 years storage at 4° C. For each CD3-specific ankyrin repeat protein, an aliquot was stored at −70° C. as a reference. The reference and the heat-stressed samples were exposed to the same number of freeze-thaw cycles before analysis. The samples were evaluated for aggregation and turbidity (size range >100 μm) by UV spectrometry and for multimerization, aggregation and fragmentation by SDS-PAGE and analytic SEC. Stressed samples were then compared to the respective reference samples (FIGS. 2, 3 and 4).

Thermal Stability Assessment of Exemplary CD3-Specific Ankyrin Repeat Proteins Using Circular Dichroism (CD) Spectroscopy

The same CD3-specific ankyrin repeat proteins were also assessed for their thermal stability and unfolding/refolding propensity using a Jasco J-815 spectrophotometer (non-stressed proteins only). The Tm (melting temperature) of the selected proteins is determined by CD as a parameter for thermal stability. In brief, the ellipticity was recorded at 222 nM and a temperature range from 20° C. to 95° C. was applied followed by reverse scan to record the refolding behavior. The Tm of the selected CD3-specific ankyrin repeat proteins is the midpoint of the protein unfolding.

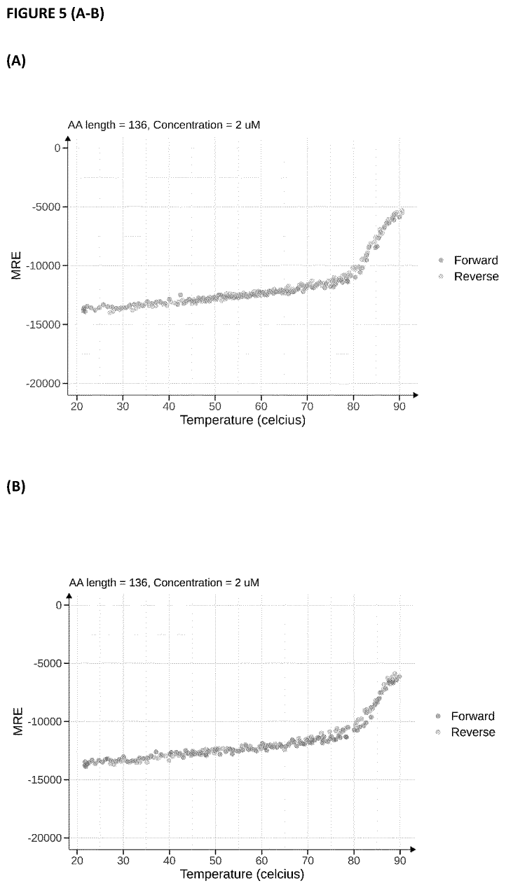

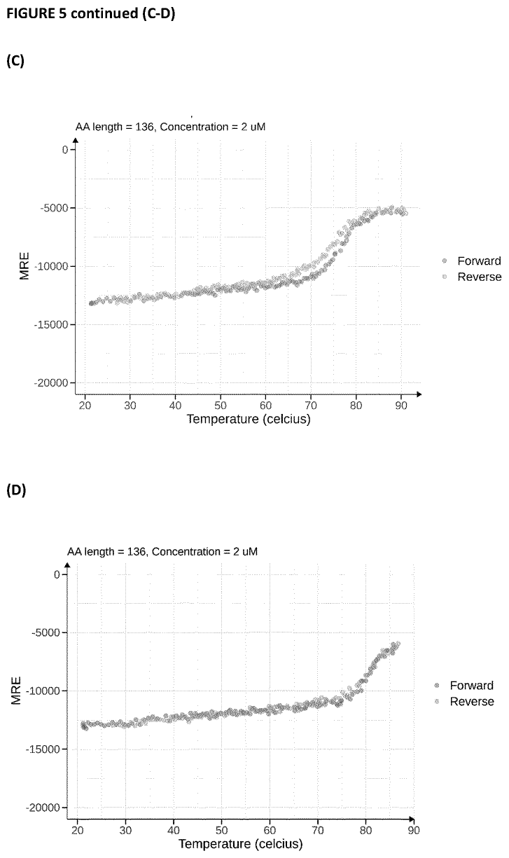

Samples were prepared at a concentration of 2 μM in TBS pH 8.0 (50 mM Tris, 500 mM NaCl) (FIG. 5). For all four tested proteins, a Tm higher than 65° C. was obtained.

TABLE 1
Tm (melting temperature) of four exemplary
CD3-specific ankyrin repeat proteins

| DARPin ® | | |
|---|---|---|
| protein # | SEQ ID NO | Tm ° C. |
| #1 | 1 | 85 |
| #2 | 2 | 82 |
| #3 | 3 | 75 |
| #4 | 4 | 83 |

Determination of the Aggregation Onset Temperature T_{agg} with Dynamic Light Scattering (DLS)

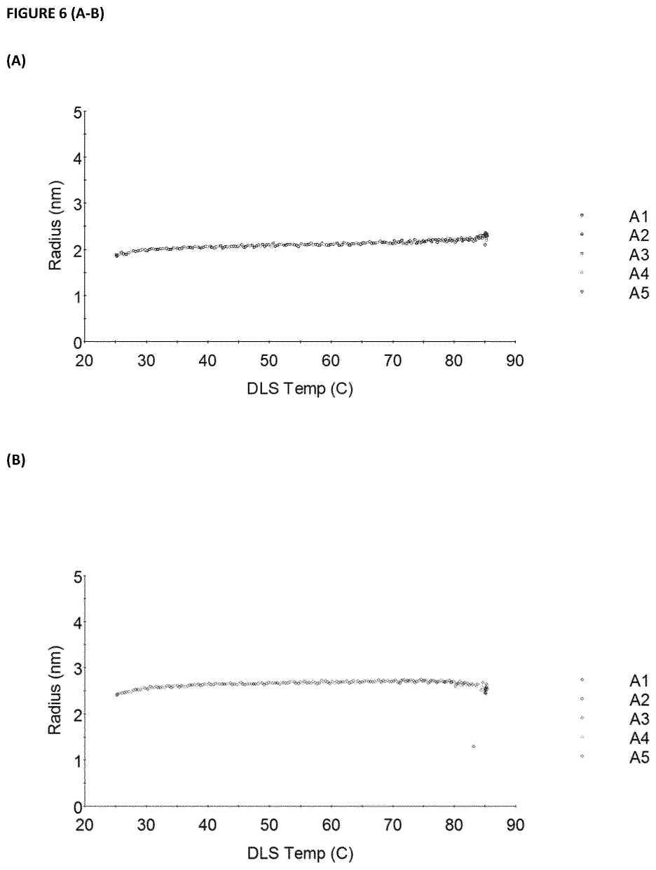

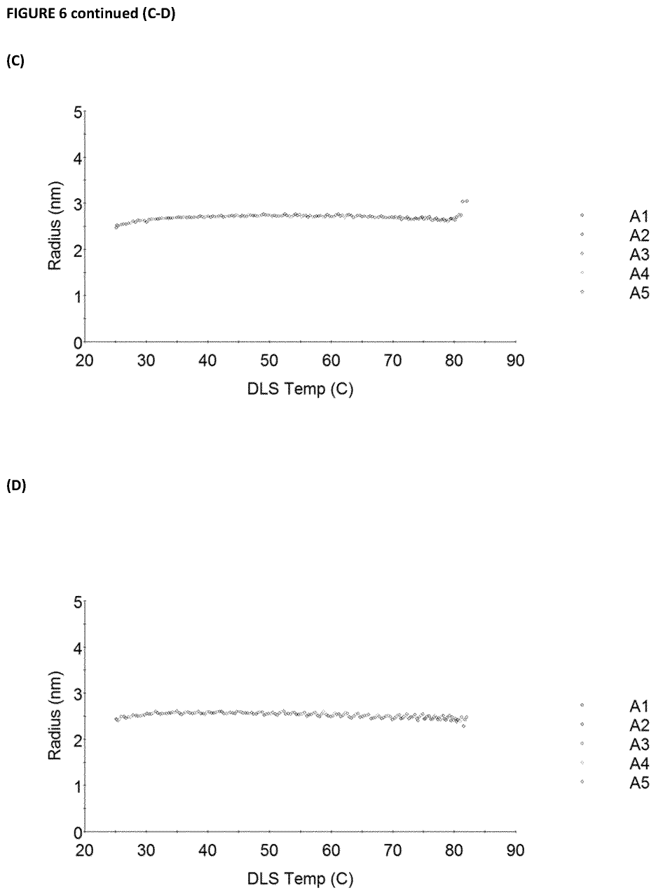

The same CD3-specific ankyrin repeat proteins were assessed for their temperature dependent aggregation propensity (T_{agg}). A temperature range of 25° C. to 85° C. was applied and plotted against the hydrodynamic radius of the proteins. For the assessment a Wyatt DynaPro Plate Reader II (serial number 140-WPR2) device was used, while measurements where processed with Dynamics 7.8.1.30 software. Ankyrin repeat samples (Table 2) were prepared at a concentration of 1 mg/ml in TBS pH 8.0 (50 mM Tris, 500 mM NaCl) and measured in 5 replicates (A1, A2, A3, A4 and A5) (FIG. 6).

As shown in FIG. 6 all four tested ankyrin repeat proteins tend to aggregate at a temperature much higher than 65° C.

TABLE 2
Tagg (temperature-dependent aggregation propensity) of
four exemplary CD3-specific ankyrin repeat proteins

| DARPin ® | | |
|---|---|---|
| protein # | SEQ ID NO | Tagg ° C. |
| #1 | 1 | >85 |
| #2 | 2 | >80 |
| #3 | 3 | >80 |
| #4 | 4 | >80 |

### Example 3: Pharmacokinetic Analysis of CD3-Specific Ankyrin Repeat Proteins in Female BALB/c Mice

In order to determine whether a CD3-specific ankyrin repeat domain of the invention can have an appropriate serum half-life in vivo for it to be useful for the development of therapeutic agents, the pharmacokinetic profiles of DARPin® protein #1, DARPin® protein #2, DARPin® protein #3 and DARPin® protein #4 were analyzed in mice. For that, DARPin constructs were subcloned and expressed as described above into derivatives of the pQE30 (Qiagen) expression vector, containing an N-terminal His-tag, an HSA binding ankyrin repeat domain for half-life extension, followed by one of the CD3-specific binding domains. The two ankyrin repeat domains were connected to each other by a peptide linker, such as the linker of SEQ ID NO: 31.

For example, expression vectors encoding the following ankyrin repeat proteins were constructed:
DARPin® protein #5 (SEQ ID NO:33 with a His-tag (SEQ ID NO: 27) fused to its N terminus);
DARPin® protein #6 (SEQ ID NO:34 with a His-tag (SEQ ID NO: 27) fused to its N terminus);
DARPin® protein #7 (SEQ ID NO:35 with a His-tag (SEQ ID NO: 27) fused to its N terminus);
DARPin® protein #8 (SEQ ID NO:36 with a His-tag (SEQ ID NO: 27) fused to its N terminus);

In Vivo Administration and Sample Collection

DARPin® protein #5, DARPin® protein #6, DARPin® protein #7 and DARPin® protein #8, formatted with a human serum albumin specific ankyrin repeat domain (SEQ ID NO: 29), were administered as a single intravenous bolus injection into the tail vein of 6 mice for each ankyrin repeat fusion protein. The target dose level was 1 mg/kg with an application volume of 5 mL/kg. Ankyrin repeat fusion proteins were formulated in phosphate-buffered saline (PBS) solution.

Mice were split into 2 groups with equal numbers of animals. Four serum samples were collected from each mouse. Blood samples for pharmacokinetic investigations were collected from the saphenous vein at 5 min, 4 h, 24 h, 48 h, 76 h, 96 h and 168 h post compound administration. Blood was kept at room temperature to allow clotting followed by centrifugation and collection of serum.

Bioanalytics by ELISA to Measure Ankyrin Repeat Proteins in Serum Samples

One hundred μl per well of 10 nM polyclonal goat anti-rabbit IgG antibody (Ab18) in PBS was coated onto a NUNC Maxisorb ELISA plate overnight at 4° C. After washing with 300 μl PBST (PBS supplemented with 0.1% Tween20) per well five times, the wells were blocked with 300 μl PBST supplemented with 0.25% Casein (PBST-C) for 1 h at room temperature (RT) on a Heidolph Titramax 1000 shaker (450 rpm). Plates were washed as described above. 100 μl 5 nmol/L rabbit anti-DARPin® 1-1-1 antibody in PBST-C was added and the plates were incubated at RT (22° C.) with orbital shaking (450 rpm) for 1 h. Plates were washed as described above.

One hundred μl of diluted serum samples (1:20-1:312500 in 1:5 dilution steps) or ankyrin repeat protein standard curve samples (0 and 50-0.0008 nmol/L in 1:3 dilution steps) were applied for 2 h, at RT, shaking at 450 rpm. Plates were washed as described above.

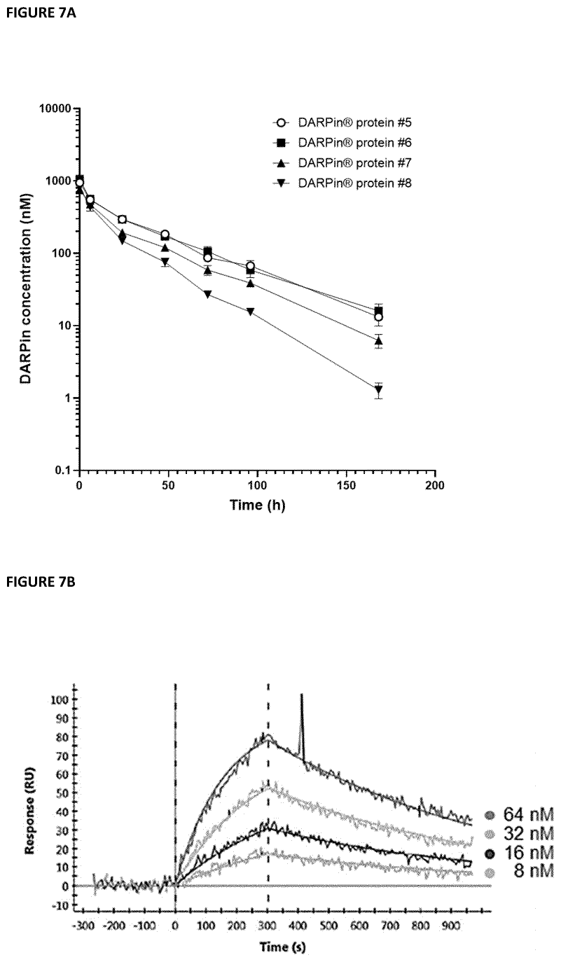

Wells were then incubated with 100 μl murine anti-RGS-His-HRP IgG (Ab06, 1:2000 in PBST-C) and incubated for 1 h, at RT, 450 rpm. Plates were washed as described above. The ELISA was developed using 100 μl/well TMB substrate solution for 5 minutes and stopped by the addition of 100 μl 1 mol/L H2504. The difference between the absorbance at 450 nm and the absorbance at 620 nm was calculated. Samples were measured in duplicate on two different plates. FIG. 7A shows the serum concentrations of DARPin® protein #5, DARPin® protein #6, DARPin® protein #7 and DARPin® protein #8 as a function of time after the single intravenous administration into mice. The traces indicate roughly mono-exponential elimination of the compounds.

Pharmacokinetic Analysis

Pharmacokinetic data analysis was performed at Molecular Partners using Version 7.0 of the WinNonlin program as part of Phoenix 64, Pharsight, North Carolina. Calculation of the pharmacokinetic parameters based on the mean concentration-time data of the animals dosed via intravenous bolus injection was performed with non-compartmental analysis (NCA model 200-202, IV bolus, linear trapezoidal linear interpolation). The following pharmacokinetic parameters were calculated: AUCinf, AUClast, AUC_% extrapol, Cmax, Tmax, Cl_pred, Vss_pred, t1/2 Maximum serum concentrations (Cmax) and the times of their occurrence (Tmax) were obtained directly from the serum concentration-time profiles. The area under the serum concentration-time curve (AUCinf) was determined by the linear trapezoidal formula up to the last sampling point (Tlast) and extrapolation to infinity assuming mono-exponential decrease of the terminal phase. The extrapolation up to infinity was performed using Clast/λz, where λz denotes the terminal rate constant estimated by log linear regression and Clast denotes the concentration estimated at Tlast by means of the terminal log-linear regression. Total serum clearance (Cl_pred) and the apparent terminal half-life were calculated as follows: Cl_pred=i.v. dose/AUCinf and t1/2=In2/λz. The steady-state volume of distribution Vss was determined by: Vss=i.v. dose AUMCinf/(AUCinf)2. AUMCinf denotes the total area under the first moment of drug concentration-time curve extrapolated to infinity using the same extrapolation procedure as described for calculation of AUCinf. To calculate PK parameters based on concentrations given in nmol/L dose values given as mg/kg were converted to nmol/kg by using the molecular weight of the ankyrin repeat proteins. Table 3 shows the summary of pharmacokinetic characteristics of the four tested ankyrin repeat proteins DARPin® protein #5, DARPin® protein #6, DARPin® protein #7 and DARPin® protein #8 following single intravenous administration of 1 mg/kg.

TABLE 3
Pharmacokinetic parameters for four exemplary HSA/CD3-specific ankyrin repeat proteins

| | | DARPin ® | DARPin ® | DARPin ® | DARPin ® |
|---|---|---|---|---|---|
| parameter | unit | protein #5 | protein #6 | protein #7 | protein #8 |
| AUCINF_pred | h*(nmol/L) | 26470 | 26814 | 18688 | 14155 |
| AUCINF_D_pred | (h*nmol*kg)/(L*mg) | 26470 | 26814 | 18688 | 14155 |
| AUClast | h*(nmol/L) | 25857 | 26060 | 18420 | 14115 |
| Cmax | nmol/L | 949 | 1042 | 761 | 820 |
| Tmax | h | 0.083 | 0.083 | 0.083 | 0.083 |
| CI_pred | L/(h*kg) | 0.00127 | 0.00126 | 0.00179 | 0.00237 |
| HL_Lambda_z (half life) | h | 32.3 | 34.3 | 29.0 | 21.0 |
| Vss_pred | L/kg | 0.0515 | 0.0515 | 0.0618 | 0.0547 |
| AUC_%Extrap_pred | (%) | 2 | 3 | 1 | 0 |
| AUC_%Back_Ext_pred | (%) | 0 | 0 | 0 | 0 |

### Example 4: Determination of Dissociation Constants (K_{D}) of Ankyrin Repeat Proteins with Binding Specificity for Human CD3 by Surface Plasmon Resonance (SPR) Analysis

The binding affinities of four purified ankyrin repeat proteins on recombinant human CD3 target were analyzed using a ProteOn instrument (BioRad) and the measurement was performed according standard procedures known to the person skilled in the art. For that, DARPin® protein #1, DARPin® protein #2 and DARPin® protein #3 and DARPin® protein #4 of the invention were subcloned and expressed as described above into derivatives of the pQE30 (Qiagen) expression vector, containing an N-terminal His-tag, two different TAAs (TAA2 and TAA3, respectively) binding ankyrin repeat domains, followed by one of the four CD3-specific ankyrin repeat protein constructs.

Briefly, biotinylated human scCD3εγ was diluted in PBST (PBS, pH 7.4 containing 0.005% Tween 20®) and coated on a NLC chip (BioRad) to a level of around 700-1400 resonance units (RU). The interaction of ankyrin repeat protein and human CD3 was then measured by injecting 300 μl running buffer (PBS, pH 7.4 containing 0.005% Tween 20®) containing serial dilutions of ankyrin repeat proteins covering a concentration range between 64 nM and 4 nM for multi-trace SPR measurements, followed by a running buffer flow for at least 10 minutes at a constant flow rate of 60 μl/min (off-rate measurement). The regeneration was performed using 30 μl of 10 mM Glycine pH 2. The signals (i.e. resonance unit (RU) values) of the interspots and a reference injection (i.e. injection of running buffer only) were subtracted from the RU traces obtained after injection of ankyrin repeat protein (double-referencing). Binding parameters (K_{D}, on-rate, off-rate) against CD3 were determined for the affinity-matured constructs. On-rates (kₒₙ) and dissociation constants (K_{D}) are given only as approximation because binding equilibrium was not appropriately reached even at highest sample concentration, leading to non-optimal fits for on-rates.

As representative example, FIG. 7B shows SPR traces obtained for DARPin® molecule #3 formatted with TAA2- and TAA3-binding designed ankyrin repeat domains. Dissociation constants (K_{D}) were calculated from the estimated on- and off-rates using standard procedures known to the person skilled in the art. KID values of the binding interactions of selected ankyrin repeat proteins with human CD3 were determined to be in the range of 6-35 nM (see Table 4).

TABLE 4
K_{D} values of ankyrin repeat protein - human CD3 interactions

| | | | | | number |
|---|---|---|---|---|---|
| | | | | | of |
| | | | | | mea- |
| | K_{D} | | kₒₙ | k_{off} | sure- |
| DARPin ® protein # | [nM] | | [1/Ms] | [1/s] | ments |
| DARPin ® protein #2 | ~35 ± 15 | nM | ~4.9E+04 | 1.5E−03 | n = 2 |
| with TAA2/TAA3 | | | | | |
| domains | | | | | |
| DARPin ® protein #3 | ~15 ± 2 | nM | ~8.9E+04 | 1.3E−03 | n = 4 |
| with TAA2/TAA3 | | | | | |
| domains | | | | | |
| DARPin ® protein #4 | ~6 ± 2 | nM | ~8.2E+04 | 4.1E−04 | n = 6 |

with TAA2/TAA3
domains

### Example 5: Determination of CD3 T Cell Binding

The determination of CD3 binding was performed with primary human T cells using Mirrorball laser scanning imaging cytometry. Therefore, primary T cells were isolated from human peripheral blood mononuclear cells (PBMCs) using a pan-T cell purification kit (Miltenyi Biotec). A titration of DARPin® protein #1, DARPin® protein #2, DARPin® protein #3 and DARPin® protein #4, formatted into a trispecific format (including TAA2 and TAA3 binding ankyrin repeat domains)—named herein as DARPin® protein #A, DARPin® protein #B, DARPin® protein #C and DARPin® protein #D, respectively—or tetraspecific format (including an additional ankyrin repeat domain binding specifically to human serum albumin) were incubated with 50′000 pan-T cells per well in presence of 600 μM human serum albumin (to mimic physiological serum concentration) for 30 minutes at 4° C. To generate the tri-specific TCE proteins, DARPin® protein #1, DARPin® protein #2, DARPin® protein #3 and DARPin® protein #4, respectively, was subcloned and expressed as described above into a derivative of the pQE30 (Qiagen) expression vector, encoding an N-terminal His-tag, followed by two different TAA-specific ankyrin repeat domains (in the order TAA2- and TAA3-specific, respectively), and then followed by one of the CD3-specific ankyrin repeat domains of the invention. For the tetra-specific TCE proteins, a human serum albumin-specific ankyrin repeat domain was additionally included between the His-tag and the TAA2-specific domain. The ankyrin repeat domains were connected to each other by a peptide linker, such as the linker of SEQ ID NO: 31. Two benchmark T-cell engagers were applied as controls, targeting either TAA2 or TAA3. After washing, CD3 binding was detected by 1:100-diluted anti-penta-His Alexa Fluor 488 antibody (Qiagen). After 30 min incubation at 4° C., cells were washed and resuspended in Cytofix fixation buffer (BD Biosciences) and counterstained by 5 μM DRAQ5 (Abcam) for 15 min at RT. Median of mean fluorescence intensities of Alexa Fluor 488 binding on far-red counterstained cells were measured by Mirrorball using Cellista software (SPT Labtech) and data was plotted using GraphPad Prism 8.

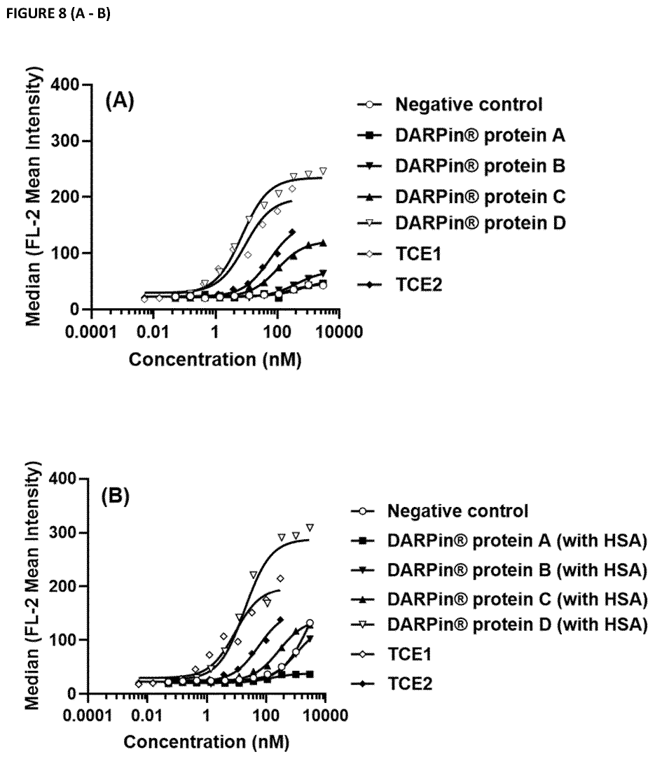

As shown in FIGS. 8A-B, DARPin® proteins show a broad range of affinities from no binding detectable for #A to binding as good as benchmark molecules for #D (known benchmark T cell engagers, TCE1 with binding specificity for TAA2 and TCE2 with binding specificity for TAA3). The CD3 binding to T cells is aligned with CD3 affinity measured by SPR. The presence of an additional HSA binding domain (see FIG. 8B) had only a minor impact on binding to T cells. Table 5 shows the CD3 binding affinity of the four exemplary ankyrin repeat proteins, as represented by their EC₅₀ values.

TABLE 5
EC₅₀ values of TAA2-TAA3-CD3-specific DARPin ® proteins

| DARPin ® protein # | EC50 [nM] | |
|---|---|---|
| #1 (A) | Too low- not calculated | |
| #2 (B) | 250-400 | nM |
| #3 (C) | 50-100 | nM |
| #4 (D) | 5-15 | nM |

### Example 6: Assessment of Target-Specific Short-Term Tumor Cell Killing Induced by CD3-Specific Ankyrin Repeat Proteins by LDH Cytotoxicity Assay

Specificity and potency of the four CD3-specific ankyrin repeat proteins (DARPin® proteins #1 to #4) in the format, described for cell binding (Example 5), were assessed by an in-vitro short-term cytotoxicity assay by LDH release.

Effector and target cells were co-incubated in duplicates in 96-well plates with an E:T ratio of 5:1 in presence of 600 μM human serum albumin (to mimic physiological concentration). Untouched T cells were isolated from human PBMCs by using a pan-T cell isolation Kit (Miltenyi). 100′000 purified pan-T cells (effector cells) and 20′000 TAA2/TAA3-expressing tumor cells (target cells) per well were incubated with serial dilutions of selected the selected CD3-specific ankyrin repeat proteins, control benchmark molecules or control containing 1% Triton X-100 for 48 hours at 37° C. After 48 h incubation, cells were spun down and 100 μl per well supernatant and 100 μl per well LDH reaction mixture (LDH detection kit; Roche Applied Science) were incubated for 30 minutes. Absorbance was measured at 492 nm-620 nm by TECAN infinite M1000Pro reader. After background correction, OD values were plotted using GraphPad Prism 8.

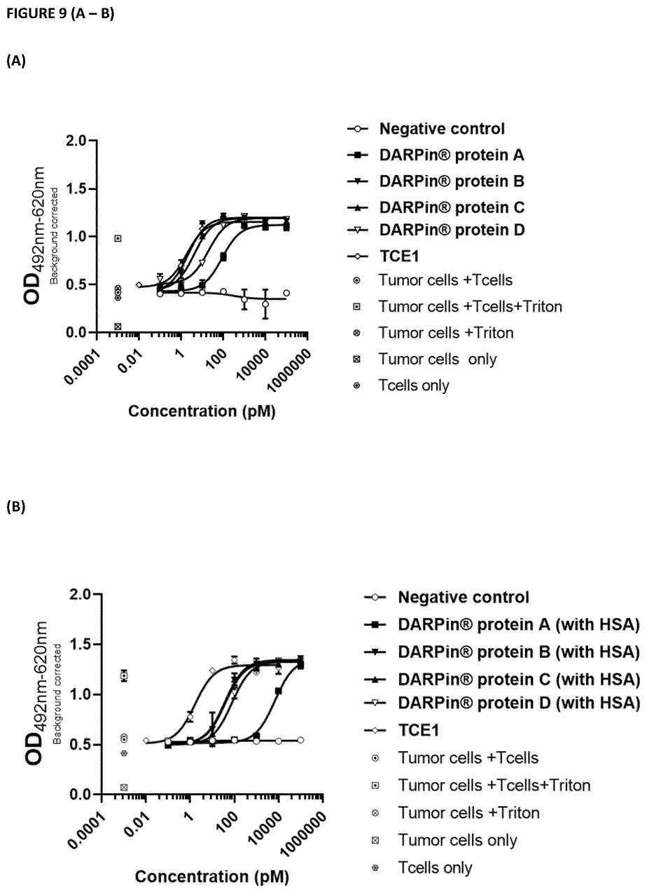

As shown in FIG. 9A, for DARPin proteins without half-life extension, DARPin® protein #2 and DARPin® protein #3 induced tumor cell killing comparable to benchmark molecule (known benchmark T cell engager, TCE1 with binding specificity for TAA2), whereas DARPin® protein #1 and DARPin® protein #4 show 10 to 100-fold lower potency. In FIG. 9B, half-life extended DARPin® proteins show about 4 to 70-fold reduction in potency compared to the corresponding non-half-life extended molecules.

### Example 7: Assessment of target-specific short-term T cell activation and IFNγ secretion

Specificity and potency of the above-described CD3-specific ankyrin repeat proteins (Examples 5 and 6) were assessed in an in vitro short-term T cell activation assay by FACS measuring CD25 activation marker on CD8+ T cells and by ELISA measuring IFNγ secretion.

Therefore, 100′000 purified pan-T cells (effector cells) and 100′000 TAA2/TAA3-expressing tumor cells (target cells) per well were co-incubated (E:T ratio 1:1) with serial dilutions of selected binding proteins or control benchmark molecules in duplicates in presence of 600 μM human serum albumin for 24 hours at 37° C. After 24 hours, 100 μl supernatant per well were transferred for measurement of IFNγ secretion by human IFNγ Standard ABTS ELISA Development Kit (PeproTech) according to manufactures protocol. Cells were washed and stained with 1:1′000 Live/Dead Aqua (Thermo Fisher), 1:250 mouse anti-human CD8 Pacific Blue (BD), and 1:100 mouse anti-human-CD25 PerCP-Cy5.5 (eBiosciences) antibodies for 30 min at 4° C. After washing and fixation, cells were analyzed on a FACS Canto II (BD) machine. T cell activation was assessed by measuring CD25+ cells on Live/Dead-negative and CD8+ gated T cells. FACS data was analyzed using FlowJo software and data was plotted using GraphPad Prism 8.

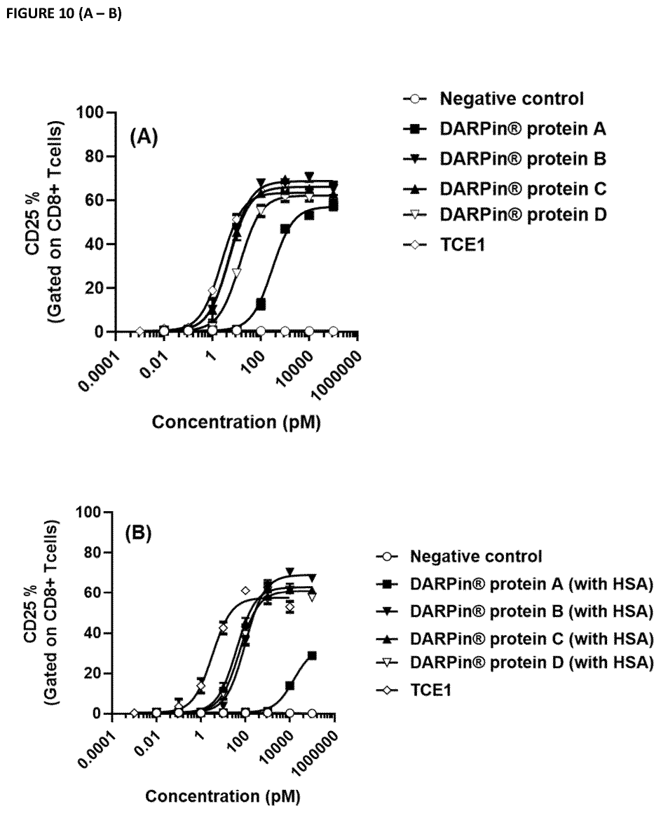

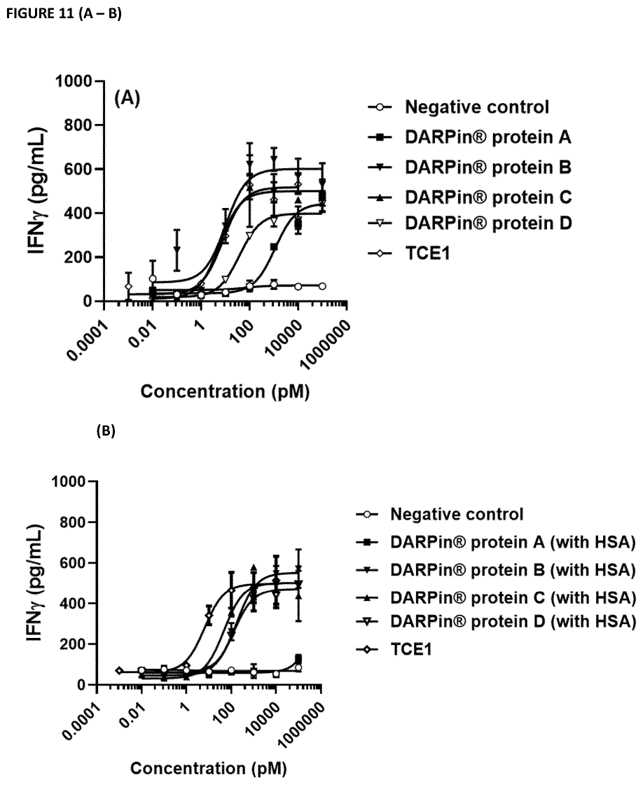

As shown in FIG. 10A, DARPin® protein #2 and DARPin® protein #3, without half-life extension, induced specific short-term T-cell activation comparable to benchmark molecules (known benchmark T cell engager, TCE1 with binding specificity for TAA2), whereas DARPin® protein #1 and DARPin® protein #4 showed 10 to 100-fold lower potency. In FIG. 10B, half-life extended DARPin® proteins showed about 3- to 100-fold reduction in potency compared to the corresponding non-half-life extended molecules.

### Example 8: Assessment of Target-Specific Long-Term Tumor Cell Killing by IncuCyte

Specificity and potency of the CD3-specific ankyrin repeat proteins (described in Examples 5, 6 and 7) were assessed by an in-vitro long-term killing assay using the IncuCyte S3 platform.

TAA2/TAA3-expressing tumor cells were first transduced with NucLight Red (NLR) lentiviral particles (Sartorius), and red-fluorescent cells selected by 0.7 μg/ml puromycin and/or FACS sorting. Long-term tumor cell killing was then assessed with the IncuCyte S3 system (Sartorius). Effector and target cells were co-incubated in duplicates on 0.01% poly-L-ornithine-coated 96-well plates with an E:T ratio of 5:1 in presence of 1:200 Annexin V green (Sartorius) and 600 μM human serum albumin (to mimic physiological concentration). 50′000 purified (Miltenyi) pan-T cells (isolated form healthy donor PBMCs)+10′000 TAA2/TAA3-expressing tumor cells (NLR transduced) per well were incubated up to 6 days at 37° C. together with serial dilutions of the selected ankyrin repeat proteins or control benchmark molecules. Images were taken every 2 h to assess for cell proliferation (red fluorescence from NLR) and cell death (green fluorescence from Annexin V). Total cell proliferation and tumor cell killing was analyzed by calculating the area under the curve after 6 days of co-culture using GraphPad Prism 8.

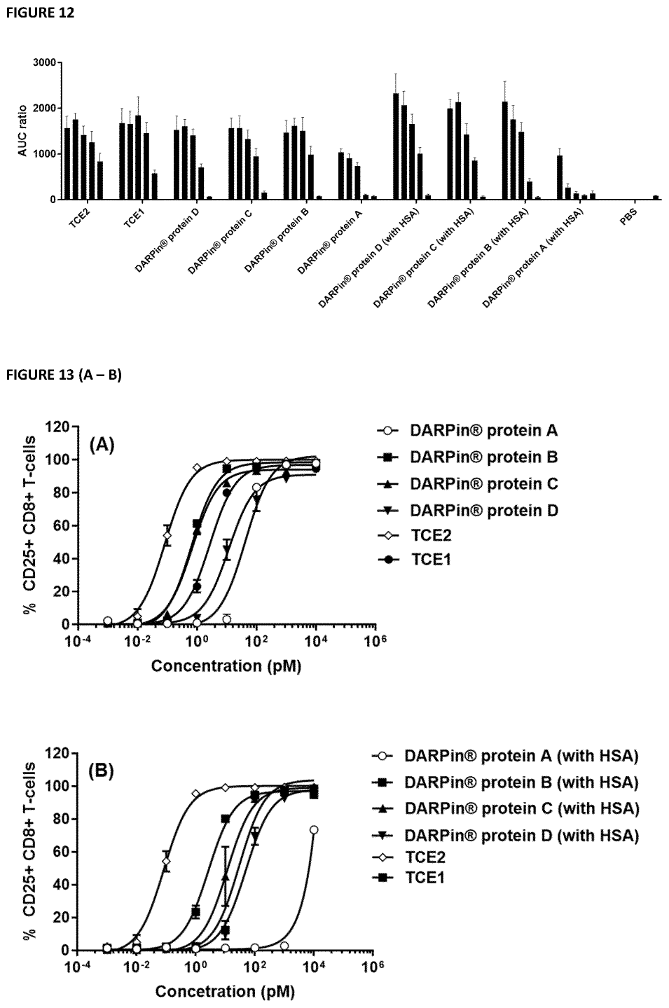

As shown in FIG. 12, tested DARPin® proteins #2, #3 and #4 show potent and specific tumor cell killing, comparable to benchmark molecules (known benchmark T cell engagers, TCE1 with binding specificity for TAA2 and TCE2 with binding specificity for TAA3), independent of half-life extension. Only the lower-affinity DARPin® #1 shows a reduction of killing potency.

### Example 9: Assessment of Target-Specific Long-Term T-Cell Activation and Proliferation by FACS

Specificity and potency of the CD3-specific ankyrin repeat proteins (described in Examples 5, 6, 7 and 8) were assessed by a FACS-based in-vitro long-term T-cell activation assay.

To assess drug- and target-specific T-cell activation and proliferation, effector and target cells were co-incubated in duplicates with an E:T ratio of 1:1 in presence of serial dilutions of selected molecules. Purified (Miltenyi) pan-T cells (isolated form healthy donor PBMCs) were first labeled with 5 μM CellTrace Violet (CTV) (Thermo Fischer) for 20 min at 37° C. 50′000 CTV-labeled pan-T cells+50′000 TAA2/TAA3-expressing tumor cells per well were then incubated at 37° C. together with serial dilutions of the selected CD3-specific ankyrin repeat proteins or control benchmark molecules TCE1 and TCE2) in presence of 600 μM human serum albumin (to mimic physiological concentration). After 5 days, cells were washed with PBS and stained with 1:5′000 Live/Dead Green (Thermo Fisher), 1:100 mouse anti-human CD8 PE (BD), and 1:100 mouse anti-human-CD25 PerCP-Cy5.5 (eBiosciences) antibodies for 30 min at 4° C. After 2 washes with PBS, cells were fixed using CelIFIX (BD) for 20 min at 4° C., and finally the buffer replaced with PBS. Stained cells were analyzed on a FACS Canto II (BD) machine. T cell activation was assessed by measuring CD25+ cells on Live/Dead-negative and CD8+ gated T cells. T-cell proliferation was assessed by gating Live/Dead-negative and CTV-positive cells. FACS data was analyzed using FlowJo software; data was plotted using GraphPad Prism 8.

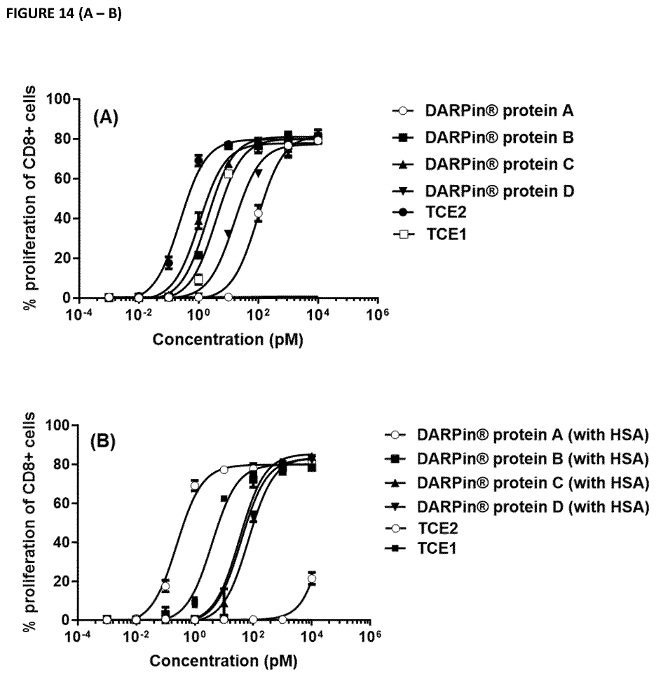

As shown in FIGS. 13 and 14, DARPin® proteins #2 and #3, without half-life extension, induced long-term T-cell activation and proliferation comparable to benchmark molecules (known benchmark T cell engagers TCE1 with binding specificity for TAA2 and TCE2 with binding specificity for TAA3), whereas DARPin® proteins #1 and #4 show 10 to 100-fold lower potency. Half-life extended CD3-specific ankyrin repeat proteins show about 5- to 30-fold reduction in potency compared to the corresponding non-half-life extended molecules.

### Example 10: In Vivo Efficacy Evaluation of Exemplary Multi-Specific TCE Binding Proteins in PBMC Humanized Mice and TAA-Expressing Tumor Model

Experiment A. DARPin® protein #2, DARPin® protein #3 and DARPin® protein #4 were formatted into tetra-specific TCE molecules, which in addition to DARPin® protein #2, DARPin® protein #3 or DARPin® protein #4 comprise ankyrin repeat domains with binding specificity for TAA2, TAA3 and human serum albumin. For that, DARPin® protein #2, DARPin® protein #3 and DARPin® protein #4 were subcloned and expressed as described above into derivatives of the pQE30 (Qiagen) expression vector, encoding an N-terminal His-tag, followed by a human serum albumin-specific ankyrin repeat domain, then followed by two different TAA-specific (TAA2- and TAA3-specific, respectively) ankyrin repeat domains, and then followed by one of the CD3-specific ankyrin repeat domains of the invention. The ankyrin repeat domains were connected to each other by a peptide linker, such as the linker of SEQ ID NO: 31. The corresponding TCE proteins are named herein DARPin® protein #F (comprising DARPin® protein #2), DARPin® protein #G (comprising DARPin® protein #3) and DARPin® protein #H (comprising DARPin® protein #4), respectively. DARPin® protein #2 was similarly formatted into a tri-specific TCE molecule, which in addition to DARPin® protein #2 comprises the ankyrin repeat domains with binding specificity for TAA2 and TAA3, but lacks the HSA-specific ankyrin repeat domain. This TCE protein is named herein DARPin® protein #E. DARPin® protein #E, DARPin® protein #F, DARPin® protein #G and DARPin® protein #H were tested in a Peripheral Blood Mononuclear Cell (PBMC) humanized mouse model bearing a TAA2/TAA3-expressing tumor cell line as solid subcutaneous tumor and compared to a known TAA3 targeted T cell engager molecule currently tested in clinical trials.

Materials and Methods

Animals: 60 female NOG mice, age of animals at study initiation 65 days (provider Taconic Biosciences)

Test and control molecules: DARPin® protein #E, DARPin® protein #F, DARPin® protein #G, and DARPin® protein #H were produced as described previously in a concentration of 5 mg/ml; control, benchmark TAA3 targeted TCE 0.59 mg/ml: provided by Evitria AG

Treatment groups: 60 mice were enrolled in the study. All animals were randomly allocated to the 8 different study groups. The date of tumor cell inoculation is denoted as day 0.

Method: Two days before the start of the experiment body weight was recorded and mice were randomized in order to have equal mean weight and similar standard deviation in each group. The mean weight was 19.6 g.

At day −2, mice were injected intraperitoneally with 5×10⁶ PBMC.

At day 0, mice were injected with 10⁶ TAA2/TAA3-expressing tumor cells subcutaneously in the right flank.

At day 5, treatment was started with intraperitoneal injections according to Table 8. The last treatment was at day 16.

Tumor measurement and weighing were performed at days 7, 10. 12, 14, 17, 19. Tumor volume was calculated according to following formula: [Length×(width)²×π]/6.

Tumor volume data were analyzed by comparing growth curves by Anova and following non-parametric Kruskal-Wallis test corrected for multiple comparison (Dunn's Test).

Tumor volumes of treatment groups has been compared to the volumes of the control group. Data from the two different PBMC donors were analyzed together and separately.

TABLE 7
Allocation of Treatment Groups and Treatment Scheme

| | Sub- | | | | |
|---|---|---|---|---|---|
| | group | No of | | | |
| Group | donor | mice | Treatment | Dose | Frequency |
| 1 | A | 5 | Vehicle PBS | — | 3 × week |
| | B | 5 | 0.05% Tween | | MON/WED/FRI |
| 2 | A | 5 | Benchmark | 200 μg/kg | daily |
| | B | 5 | TAA3 targeted | | |
| | | | TCE | | |
| 3 | A | 5 | DARPin ® | 200 μg/kg | daily |
| | B | 5 | protein #E | | |
| 4 | A | 5 | DARPin ® | 200 μg/kg | 3 × week |
| | B | 5 | protein #F | | MON/WED/FRI |
| 5 | A | 5 | DARPin ® | 200 μg/kg | 3 × week |
| | B | 5 | protein #G | | MON/WED/FRI |
| 6 | A | 5 | DARPin ® | 200 μg/kg | 3 × week |
| | B | 5 | protein #H | | MON/WED/FRI |

Results

Tumor Growth Inhibition

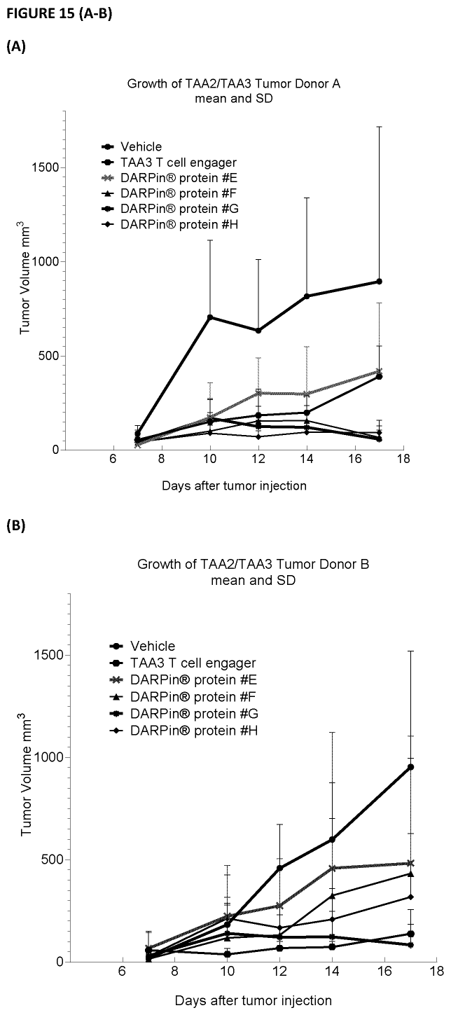

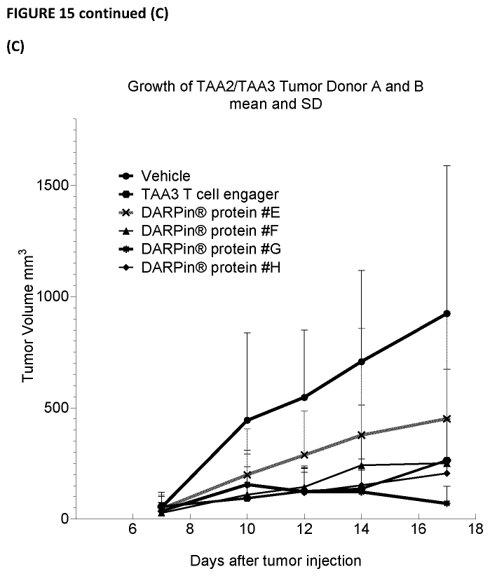

The tumor growth curves upon exposure to the tested multi-specific binding proteins and the known benchmark T cell engager is summarized in FIG. 15A-C, including both donors together and separately.

Tables 8 (both donors), 9 (Donor A) & 10 (Donor B): Statistics of tumor growth
Both Donors together

| Days after | | TAA3 T cell | | DARPin ® | | DARPin ® | | DARPin ® | | DARPin ® | |
|---|---|---|---|---|---|---|---|---|---|---|---|
| initiation of | Vehicle | engager | | protein #E | | protein #F | | protein #G | | protein #H | |
| Treatment | mean | mean | p | mean | p | mean | p | mean | p | mean | p |
| 2 | 52.1 | 55.0 | ns | 46.1 | ns | 27.4 | ns | 36.7 | ns | 34.4 | ns |
| 5 | 443.4 | 93.0 | ++ | 197.7 | ns | 109.0 | + | 154.1 | ns | 150.9 | ns |
| 7 | 546.3 | 124.8 | ++ | 287.2 | ns | 142.3 | + | 122.1 | ++ | 118.3 | ++ |
| 9 | 706.8 | 135.6 | +++ | 376.8 | ns | 240.5 | + | 121.1 | +++ | 150.6 | ++ |
| 12 | 923.4 | 263.0 | ns | 450.3 | ns | 250.2 | ++ | 69.9 | ++++ | 204.6 | ++ |

Donor A

| Days after | | TAA3 T cell | | DARPin ® | | DARPin ® | | DARPin ® | | DARPin ® | |
|---|---|---|---|---|---|---|---|---|---|---|---|
| initiation of | Vehicle | engager | | protein #E | | protein #F | | protein #H | | protein #G | |
| Treatment | mean | mean | p | mean | p | mean | p | mean | p | mean | p |
| 2 | 86.1 | 53.0 | ns | 26.8 | ns | 41.0 | ns | 43.1 | ns | 43.2 | ns |
| 5 | 704.6 | 149.5 | +++ | 172.0 | ++ | 100.3 | +++ | 169.1 | ++ | 88.6 | +++ |
| 7 | 634.0 | 183.0 | ++ | 300.5 | + | 153.2 | ++ | 124.0 | +++ | 69.2 | +++ |
| 9 | 815.7 | 197.8 | ++ | 296.7 | + | 156.4 | ++ | 119.5 | +++ | 93.7 | +++ |
| 12 | 895.1 | 389.3 | ns | 418.1 | ns | 67.6 | ++ | 57.4 | ++ | 91.8 | + |

Donor B

| Days after | | TAA3 T cell | | DARPin ® | | DARPin ® | | DARPin ® | | DARPin ® | |
|---|---|---|---|---|---|---|---|---|---|---|---|
| initiation of | Vehicle | engager | | protein #E | | protein #F | | protein #G | | protein #H | |
| Treatment | mean | mean | p | mean | p | mean | p | mean | p | mean | p |
| 2 | 18.1 | 57.1 | ns | 65.5 | ns | 13.8 | ns | 30.4 | ns | 25.7 | ns |
| 5 | 182.1 | 36.6 | ns | 223.5 | ns | 117.8 | ns | 139.1 | ns | 213.1 | ns |
| 7 | 458.7 | 66.6 | + | 273.9 | ns | 131.4 | ns | 120.2 | + | 167.5 | ns |
| 9 | 597.9 | 73.5 | + | 456.8 | ns | 324.6 | ns | 122.7 | + | 207.6 | ns |
| 12 | 951.8 | 136.7 | ns | 482.5 | ns | 432.8 | ns | 82.3 | ++ | 317.3 | ns |

+ = p < 0.1;
** = p < 0.05;
+++ = p < 0-01;
++++ = p < 0.001;
ns = not significant

PBMC from Donor B led to lower amounts of CD8 positive lymphocytes in mouse blood. In general, antitumoral effect was observed in all tested molecules. DARPin® protein #F and the known TAA3 T cell engager showed an effect starting from 5 days after initiation of the treatment, whereas the effect of DARPin® protein #G and DARPin® protein #H was significant and relevant starting from 7 days after initiation of the treatment. When only subgroups A where compared, a statistically significant effect was observed for all tested molecules starting from day 5 after initiation of the treatment. The effect of DARPin® protein #F, DARPin® protein #G and DARPin® protein #H was stronger than that of the benchmark TAA3 T cell engager and DARPin® protein #E. When subgroups B were compared, a significant antitumoral effect was observed for DARPin® protein #G and the benchmark TAA3 T cell engager.

The result obtained with the benchmark TCE validated the model used. At the same time, a less pronounced effect of the benchmark observed in mice humanized with PBMC from donor B indicates that the sensitivity of mice belonging to this subgroup is lower. This is also reflected by the significantly lower amounts of human CD8 positive lymphocytes found at the end of the experiment in mice humanized with PBMC from donor B.

Thus, more relevance should be attributed to the data raised from mice humanized with PBMC from donor A.

The high variability in tumor size is due to the fact that no randomization and range selection has been performed in mice, because the treatment was initiated before tumors were recognizable in all mice. Despite this high variability, statistical analysis could be performed without need of normalization to the initial tumor volume.

We confirmed that all multi-specific binding proteins tested show antitumoral activity and the half-life extended molecules, DARPin® protein #F, DARPin® protein #G and DARPin® protein #H, at the doses and application regimen used, have stronger antitumoral activity than the not half-life extended DARPin® protein #E. Without wishing to be bound by theory, it is thought that the exposure of DARPin® protein #E may be lower due to the shorter half-life of this molecule.

Overall, all four tested multi-specific binding proteins showed in vivo antitumoral activity comparable or stronger to that of the benchmark T cell engager. These results demonstrated that CD3-specific ankyrin repeat domains of the invention can be combined with TAA-specific binding domain(s) (and a half-life extending moiety) to form T cell engager molecules with anti-tumor activity in vivo. Thus, when formatted in such TCE molecules, the CD3-specific ankyrin repeat domains of the invention maintain their ability to bind to CD3 and activate T cells in vivo.

Experiment B. In a similar experimental set up, DARPin® protein #3 was formatted into a hexa-specific TCE molecule, which additionally comprises three designed ankyrin repeat domains with binding specificity for TAA2, TAA3 and TAA4, respectively, and two designed ankyrin repeat domains with binding specificity for human serum albumin. For that, DARPin® protein #3 was subcloned and expressed as described above into a derivative of the pQE30 (Qiagen) expression vector, encoding an N-terminal His-tag, followed by two human serum albumin-specific ankyrin repeat domains, then followed by three different TAA-specific ankyrin repeat domains (in the order TAA3-, TAA2- and TAA4-specific, respectively), and then followed by one of the CD3-specific ankyrin repeat domains of the invention (DARPin® protein #3). The ankyrin repeat domains were connected to each other by a peptide linker, such as the linker of SEQ ID NO: 31. This hexa-specific TCE molecule is described in greater detail in U.S. patent application No. 63/265,184 filed on Dec. 9, 2021, the disclosure of which is incorporated herein by reference in its entirety. DARPin® protein #3 in such multi-specific TCE format was tested in a Peripheral Blood Mononuclear Cell (PBMC) humanized mouse model bearing a TAA2/TAA3/TAA4-expressing tumor cell line (Molm-13) as solid subcutaneous tumor and compared to a known TAA3 targeted T cell engager molecule currently tested in clinical trials.

In brief, the in vivo experiments were performed in 6 to 9-week-old female immunodeficient NXG mice (provided by Janvier Labs). Mice were maintained under standardized environment conditions in standard rodent micro-isolator cages (20+/−1° C. room temperature, 50+/−10% relative humidity and 12 hours light dark cycle). Mice received irradiated food and bedding and 0.22 um filtered drinking water. All experiments were done according to the Swiss Animal Protection Law with authorization from the cantonal and federal veterinary authorities.

NXG mice were injected intraperitoneally with hPBMC before the xenograft of the cancer cells. TAA2/TAA3/TAA4-expressing tumor cells were xenografted subcutaneously (s.c.) on the right flank area into NXG mice. Two hPBMC donors were used. Treatments were injected intravenously (i.v.) four days after cancer cells implantation. Most of tumor were not yet established at this time point. Treatments were administrated as follows:
DARPin® protein #3 (in the multi-specific TCE format) was administrated i.v. three times per week for 2 weeks at 0.5 mg/kg
benchmark TAA3 targeted T cell engager was administrated i.v. daily for 2 weeks at 0.5 mg/kg

Tumor size was evaluated by caliper measurement. Tumor volumes were calculated using the following formula: tumor volume (mm³)=0.5×length×width².

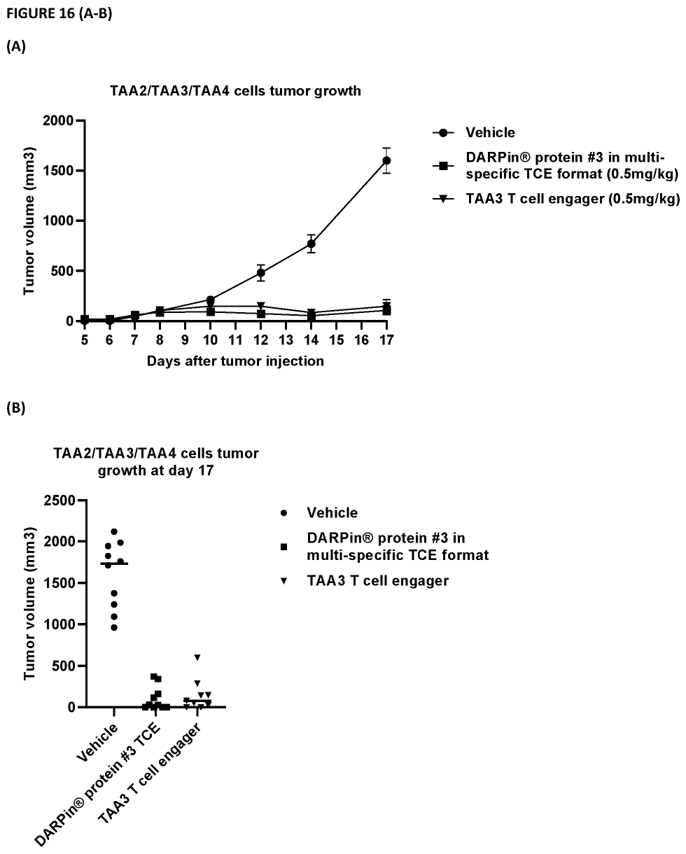

As it can be seen in FIG. 16 (A-B), DARPin® protein #3 in multi-specific TCE format showed good efficacy in terms of inhibition of tumor growth and tumor volume over the entire time of the experiment (FIG. 16A) and at 17 days after the first injection (FIG. 16B). This result further demonstrated that the CD3-specific ankyrin repeat domains of the invention can be combined with TAA-specific binding domain(s) and half-life extending moietie(s) to form T cell engager molecules with anti-tumor activity in vivo. Thus, when formatted in such TCE molecules, the CD3-specific ankyrin repeat domains of the invention maintain their ability to bind to CD3 and activate T cells in vivo.

The specification is most thoroughly understood in light of the teachings of the references cited within the specification. The aspects within the specification provide an illustration of aspects of the invention and should not be construed to limit the scope of the invention. The skilled artisan readily recognizes that many other aspects are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific aspects of the invention described herein. Such equivalents are intended to be encompassed by the following claims.

1. A recombinant binding protein comprising an ankyrin repeat domain with binding specificity for CD3, wherein said ankyrin repeat domain comprises an ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids.

2. The binding protein of claim 1, wherein said ankyrin repeat module is a first ankyrin repeat module and wherein said ankyrin repeat domain further comprises a second ankyrin repeat module having an amino acid sequence selected from the group consisting of (1) SEQ ID NOs: 5 to 9 and (2) sequences in which up to 9 amino acids in any of SEQ ID NOs: 5 to 9 are substituted by other amino acids.

3. The binding protein of claim 2, wherein said first ankyrin repeat module comprises an amino acid sequence selected from the group consisting of (1) SEQ ID NO: 5 and (2) sequences in which up to 9 amino acids in SEQ ID NO: 5 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids.

4. The binding protein of claim 2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 6 and (2) sequences in which up to 9 amino acids of SEQ ID NO: 6 are substituted by other amino acids.

5. The binding protein of claim 2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 8 and (2) sequences in which up to 9 amino acids of SEQ ID NO: 8 are substituted by other amino acids.

6. The binding protein of claim 2, wherein said first ankyrin repeat module comprises an amino acid sequence selected from the group consisting of (1) SEQ ID NO: 7 and (2) sequences in which up to 9 amino acids in SEQ ID NO: 7 are substituted by other amino acids, and wherein said second ankyrin repeat module comprises an amino acid sequence selected from the group consisting of (1) SEQ ID NO: 9 and (2) sequences in which up to 9 amino acids of SEQ ID NO: 9 are substituted by other amino acids.

7. The binding protein of claim 5, wherein said first ankyrin repeat module is located N-terminally of said second ankyrin repeat module within said ankyrin repeat domain.

8. A recombinant binding protein comprising an ankyrin repeat domain with binding specificity for CD3, wherein said ankyrin repeat domain comprises an amino acid sequence with at least 80% amino acid sequence identity with any one of SEQ ID NOs: 1 to 4, and wherein A at the second last position of SEQ ID NOs: 1 to 4 is optionally substituted by L, and/or A at the last position of SEQ ID NOs: 1 to 4 is optionally substituted by N.

9. The binding protein of claim 1, wherein said binding protein binds human CD3 in PBS with a dissociation constant (K_{D}) below 10⁻⁷ M.

10. The binding protein of claim 1, wherein said binding protein binds human CD3 on T cells with an EC₅₀ ranging from 2 to 900 nM.

11. The binding protein of claim 1, wherein said binding protein further comprises a binding agent with binding specificity for a disease-associated antigen.

12. The binding protein of claim 1, wherein said binding protein further comprises a half-life extending moiety.

13. A nucleic acid encoding the binding protein of claim 1.

14. A pharmaceutical composition comprising the binding protein of claim 8, and optionally a pharmaceutically acceptable carrier and/or diluent.

15. A method of treating a medical condition, the method comprising the step of administering to a patient in need thereof a therapeutically effective amount of the binding protein of claim 8.

16. The binding protein of claim 8, wherein said binding protein binds human CD3 in PBS with a dissociation constant (K_{D}) below 10⁻⁷ M.

17. The binding protein of claim 8, wherein said binding protein binds human CD3 on T cells with an EC 50 ranging from 2 to 900 nM.

18. The binding protein of claim 8, wherein said binding protein further comprises a binding agent with binding specificity for a disease-associated antigen.

19. The binding protein of claim 8, wherein said binding protein further comprises a half-life extending moiety.

20. A nucleic acid encoding the binding protein of claim 8.


SEQUENCE LISTING (46 sequences)

SEQ ID NO: 1 - protein, 124 aa, Artificial Sequence
  ankyrin repeat domain specific for CD3
DLGQKLLEAAWAGQDDEVRELLKAGADVNAKDSQGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 2 - protein, 124 aa, Artificial Sequence
  Ankyrin repeat domain specific for CD3
DLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 3 - protein, 124 aa, Artificial Sequence
  Ankyrin repeat domain specific for CD3
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 4 - protein, 124 aa, Artificial Sequence
  Ankyrin repeat domain specific for CD3
DLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 5 - protein, 33 aa, Artificial Sequence
  Ankyrin repeat module 1
KDSQGWTPLHTAAQTGHLEIFEVLLKAGADVNA

SEQ ID NO: 6 - protein, 33 aa, Artificial Sequence
  Ankyrin repeat module 2
KDDKGVTPLHLAAALGHLEIVEVLLKAGADVNA

SEQ ID NO: 7 - protein, 33 aa, Artificial Sequence
  Ankyrin repeat module 3
KNSRGWTPLHTAAQTGHLEIFEVLLKAGADVNA

SEQ ID NO: 8 - protein, 33 aa, Artificial Sequence
  Ankyrin repeat module 4
KNDKRVTPLHLAAALGHLEIVEVLLKAGADVNA

SEQ ID NO: 9 - protein, 33 aa, Artificial Sequence
  Ankyrin repeat module 5
KTNKRVTPLHLAAALGHLEIVEVLLKAGADVNA

SEQ ID NO: 10 - protein, 30 aa, Artificial Sequence
  N -capping module 1
DLGQKLLEAAWAGQDDEVRELLKAGADVNA

SEQ ID NO: 11 - protein, 30 aa, Artificial Sequence
  N -capping module 2
DLGQKLLEAAWAGQLDEVRILLKAGADVNA

SEQ ID NO: 12 - protein, 30 aa, Artificial Sequence
  N -capping module 3
DLGQKLLEAAWAGQDDEVRILLKAGADVNA

SEQ ID NO: 13 - protein, 30 aa, Artificial Sequence
  N -capping module 4
DLGQKLLEAAWAGQDDEVRILLAAGADVNA

SEQ ID NO: 14 - protein, 30 aa, Artificial Sequence
  N -capping module 5
DLGQKLLEAAWAGQLDEVRELLKAGADVNA

SEQ ID NO: 15 - protein, 30 aa, Artificial Sequence
  N -capping module 6
DLGQKLLEAAWAGQLDEVRILLAAGADVNA

SEQ ID NO: 16 - protein, 30 aa, Artificial Sequence
  N- capping module randomized
  VARIANT (4)..(4): xaa can be any naturally occuring amino acid
  VARIANT (5)..(5): xaa can be any naturally occuring amino acid
  VARIANT (11)..(11): xaa can be any naturally occuring amino acid
  VARIANT (12)..(12): xaa can be any naturally occuring amino acid
  VARIANT (17)..(17): xaa can be any naturally occuring amino acid
  VARIANT (20)..(20): xaa can be any naturally occuring amino acid
  VARIANT (22)..(22): xaa can be any naturally occuring amino acid
  VARIANT (23)..(23): xaa can be any naturally occuring amino acid
  VARIANT (24)..(24): xaa can be any naturally occuring amino acid
DLGXXLLQAAXXGQLDXVRXLXXXGADVNA

SEQ ID NO: 17 - protein, 28 aa, Artificial Sequence
  C-capping module 1
QDSWGTTPADLAAKYGHEDIAEVLQKAA

SEQ ID NO: 18 - protein, 28 aa, Artificial Sequence
  C-capping module 2
RDSWGTTPADLAAKYGHQDIAEVLQKAA

SEQ ID NO: 19 - protein, 28 aa, Artificial Sequence
  C-capping module 3
RDTWGTTPADLAAKYGHRDIAEVLQKAA

SEQ ID NO: 20 - protein, 28 aa, Artificial Sequence
  C-capping module 4
QDSWGTTPADLAAKYGHEDIAEVLQKLA

SEQ ID NO: 21 - protein, 28 aa, Artificial Sequence
  C-capping module 5
QDSWGTTPADLAAKYGHEDIAEVLQKLN

SEQ ID NO: 22 - protein, 28 aa, Artificial Sequence
  C-capping module 6
RDSWGTTPADLAAKYGHQDIAEVLQKLA

SEQ ID NO: 23 - protein, 28 aa, Artificial Sequence
  C-capping module 7
RDSWGTTPADLAAKYGHQDIAEVLQKLN

SEQ ID NO: 24 - protein, 28 aa, Artificial Sequence
  C-capping module 8
RDTWGTTPADLAAKYGHRDIAEVLQKLA

SEQ ID NO: 25 - protein, 28 aa, Artificial Sequence
  C-capping module 9
RDTWGTTPADLAAKYGHRDIAEVLQKLN

SEQ ID NO: 26 - protein, 28 aa, Artificial Sequence
  C-capping module randomized
  VARIANT (1)..(1): Xaa can be any naturally occuring amino acid
  VARIANT (3)..(3): Xaa can be any naturally occuring amino acid
  VARIANT (4)..(4): Xaa can be any naturally occuring amino acid
  VARIANT (6)..(6): Xaa can be any naturally occuring amino acid
  VARIANT (11)..(11): Xaa can be any naturally occuring amino acid
  VARIANT (15)..(15): Xaa can be any naturally occuring amino acid
  VARIANT (19)..(19): Xaa can be any naturally occuring amino acid
  VARIANT (22)..(22): Xaa can be any naturally occuring amino acid
  VARIANT (26)..(26): Xaa can be any naturally occuring amino acid
XDXXGXTPADXAARXGHQXIAXVLQXAA

SEQ ID NO: 27 - protein, 12 aa, Artificial Sequence
  His tag
MRGSHHHHHHGS

SEQ ID NO: 28 - protein, 124 aa, Artificial Sequence
  Ankyrin repeat domain specific for human serum albumin 1
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 29 - protein, 124 aa, Artificial Sequence
  Ankyrin repeat domain specific for human serum albumin 2
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 30 - protein, 124 aa, Artificial Sequence
  Ankyrin repeat domain specific for human serum albumin 3
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 31 - protein, 22 aa, Artificial Sequence
  PT linker
GSPTPTPTTPTPTPTTPTPTPT

SEQ ID NO: 32 - protein, 222 aa, Artificial Sequence
  scCD3_Avi-Bio
MQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMGGGLNDIFEAQKIEWHE

SEQ ID NO: 33 - protein, 272 aa, Artificial Sequence
  Ankyrin repeat protein specific for HSA-CD3
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGQKLLEAAWAGQDDEVRELLKAGADVNAKDSQGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 34 - protein, 272 aa, Artificial Sequence
  Ankyrin repeat protein specific for HSA-CD3
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 35 - protein, 272 aa, Artificial Sequence
  Ankyrin repeat protein specific for HSA-CD3
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 36 - protein, 272 aa, Artificial Sequence
  Ankyrin repeat protein specific for HSA-CD3
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 37 - DNA, 417 bp, Artificial Sequence
  Nucleic acid encoding domain of SEQ ID NO 1
atgagaggatcgcatcaccatcaccatcacggctcagatctgggtcaaaagttattggaggcagcttgggctggacaagatgacgaagtacgtgaattgttaaaagcgggagcagatgtaaacgcaaaagatagtcaaggttggactccgttacacacagcagcgcaaaccggccacctggaaattttcgaggtgttattgaaggctggagcagatgtgaatgcaaaagacgacaaaggggtgactccgctgcatctggcagcggcgttggggcacttggaaatcgttgaggtccttctgaaagcaggcgctgatgtgaatgcgcaagactcctggggaaccacaccagcggacctggcggctaagtacggccacgaagatattgctgaagttctgcagaaggcagcataatgatag

SEQ ID NO: 38 - DNA, 417 bp, Artificial Sequence
  Nucleic acid encoding domain of SEQ ID NO 2
atgagaggatcgcatcaccatcaccatcacggctcagatctgggtcaaaagttgttggaagctgcctgggcgggacaggatgatgaggtgcgcgaattacttaaggcgggagcagacgtgaatgcgaaaaactctcgtggctggacaccactgcacacggccgcgcaaactggtcaccttgaaattttcgaagtgcttctgaaggcaggcgcagatgtaaacgccaaggatgacaaaggggtaacaccgcttcatctggctgctgcactgggacatcttgagattgtcgaagtactgcttaaggcaggtgctgacgtaaacgctcaggattcatgggggaccacaccggcggacctggcggctaaatacggacatgaagatattgctgaagttctgcagaaggcagcataatgatag

SEQ ID NO: 39 - DNA, 417 bp, Artificial Sequence
  Nucleic acid encoding domain of SEQ ID NO 3
atgagaggatcgcatcaccatcaccatcacggctcagatctgggtcaaaagctgttggaagccgcgtgggcgggtcaggacgatgaagtccgtgagctgcttaaagcaggagccgacgtgaacgcgaagaactcacgcgggtggacgccacttcacacggccgcgcagacaggtcaccttgaaatctttgaggttcttctgaaggcaggagcagacgttaacgccaaaaacgacaagcgcgtgactccgttgcaccttgccgcagctctggggcatttggagatcgttgaggtactgttgaaagcgggagcagatgttaatgctcgcgacagttgggggacgacaccagcagacctggccgcaaaatacggacaccaagacattgctgaagttctgcagaaggcagcataatgatag

SEQ ID NO: 40 - DNA, 417 bp, Artificial Sequence
  Nucleic acid encoding domain of SEQ ID NO 4
atgagaggatcgcatcaccatcaccatcacggctcagatctgggtcaaaaattgttagaggcagcctgggcgggacagttagacgaggttcgtattcttttgaaagctggtgcggatgtgaacgcaaagaattctcgtggatggactccgttgcacaccgccgcacagactgggcacttggaaatctttgaggttctgttaaaagcaggggcagatgttaacgctaaaactaataaacgtgtcaccccccttcacctggctgcggctttaggccatttagaaatcgtggaagtattacttaaagccggggctgacgttaacgcccgtgacacttgggggacaacccctgcggatctggccgccaaatatggtcaccgcgacattgctgaagttctgcagaaggcagcataatgatag

SEQ ID NO: 41 - protein, 5 aa, Artificial Sequence
  Consensus GS linker
  VARIANT (1)..(5): [Gly-Gly-Gly-Gly-Ser]n, wherein n is 1, 2, 3, 4, 5, or 6
GGGGS

SEQ ID NO: 42 - protein, 24 aa, Artificial Sequence
  PT-rich peptide linker (standard)
GSPTPTPTTPTPTPTTPTPTPTGS

SEQ ID NO: 43 - protein, 38 aa, Artificial Sequence
  Linker L
GSPTPTPTTPTPTPTTPTPTPTTPTPTPTTPTPTPTGS

SEQ ID NO: 44 - protein, 18 aa, Artificial Sequence
  Linker S
GSPTPTPTTPTPTPTTGS

SEQ ID NO: 45 - protein, 11 aa, Artificial Sequence
  Linker XS
GSPTPTPTTGS

SEQ ID NO: 46 - protein, 6 aa, Artificial Sequence
  Linker XXS
GSPTGS